(19) United States
(12) Patent Application Publication
(10) Pub. No.: US 2004/0053786 A1
(43) Pub. Date: Mar. 18, 2004

(54) Insecticidal 1,8-naphthalenedicarboxamides

(76) Inventors: Thomas Paul Selby, Wilmington (DE); King-Mo Sun, Hockessin (DE)

Correspondence Address: Linda D Birch, E I du Pont de Nemours & Company, Legal Patents, Wilmington, DE, 19898, US

(21) Appl. No.: 10/398,638

(22) PCT Filed: Oct. 11, 2001
(86) PCT No.: PCT/US01/42632

(30) Foreign Application Priority Data
Oct. 17, 2000 (US) 60240890

(51) Int. Cl. A01N 43/40; A01N 37/18; A01N 43/36; C07D 211/06; C 07D 2 7/22; C07C 327/48; C07C 255/56; C07C 237/48

(52) U.S. Cl. 504/249; 504/283; 504/335; 546/226; 546/205; 548/530; 564/74; 564/155; 564/156

(57) ABSTRACT

Compounds of Formula I and Formula II, and their N-oxides and agriculturally suitable salts, are disclosed that are useful for controlling invertebrate pests, Formula (I), Formula (II) wherein A, B, J, R1, R2, R3, R4, and n are as defined in the disclosure. Also disclosed are compositions comprising the compounds of Formula I or Formula II and methods for controlling invertebrate pests that involve contacting the invertebrate pests or their environment with an effective amount of a compound of Formula (I) or Formula (II).

### BACKGROUND OF THE INVENTION

This invention relates to certain 1,8-naphthalenedicarboxamides, their agriculturally suitable salts and compositions, and methods of their use for controlling invertebrate pests in both agronomic and nonagronomic environments.

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, livestock, household, and public and animal health is also important. Many products are commercially available for these purposes, but the need continues for new compounds that are more effective, less costly, less toxic, environmentally safer or have different modes of action.

EP919542 discloses phthalic diamides of Formula i as insecticides

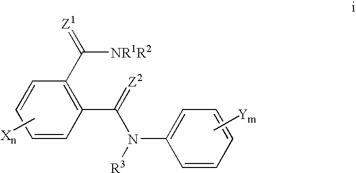

wherein, among others, Z¹ and Z² are O or S; and R¹, R² and R³ are, among others, H, alkyl or substituted alkyl.

### SUMMARY OF THE INVENTION

This invention is directed to compounds of Formula I and Formula II including all geometric and stereoisomers, N-oxides and agriculturally suitable salts thereof:

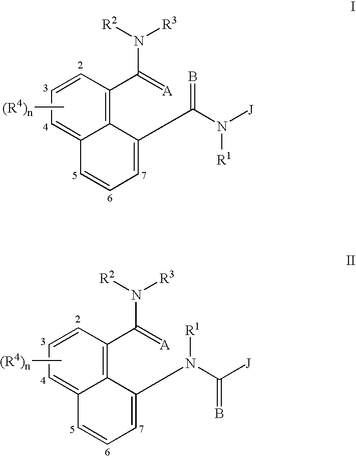

wherein

each J is independently a phenyl ring, a naphthyl ring system, a 5- or 6-membered heteroaromatic ring or an aromatic 8-, 9- or 10-membered fused heterobicyclic ring system wherein each ring or ring system is optionally substituted with 1 to 5 R⁵;

A and B are independently O or S;

n is 0 to 4;

R¹ is H; or C₁-C₆ alkyl, C₂-C₆ alkenyl, C₂-C₆ alkynyl or C₃-C₆ cycloalkyl each optionally substituted with one or more substituents selected from the group consisting of halogen, CN, NO₂, hydroxy, C₁-C₄ alkoxy, C₁-C₄ alkylthio, C₁-C₄ alkylsulfinyl, C₁-C₄ alkylsulfonyl, C₂-C₄ alkoxycarbonyl, C₁-C₄ alkylamino, C₂-C₈ dialkylamino and C₃-C₆ cycloalkylamino; or

R¹ is C₂-C₆ alkylcarbonyl, C₂-C₆ alkoxycarbonyl, C₂-C₆ alkylaminocarbonyl or C₃-C₈ dialkylaminocarbonyl;

R² is H, C₁-C₆ alkyl, C₂-C₆ alkenyl, C₂-C₆ alkynyl, C₃-C₆ cycloalkyl, C₁-C₄ alkoxy, C₁-C₄ alkylamino, C₂-C₈ dialkylamino, C₃-C₆ cycloalkylamino, C₂-C₆ alkoxycarbonyl or C₂-C₆ alkylcarbonyl;

R³ is H; or C₁-C₆ alkyl, C₂-C₆ alkenyl, C₂-C₆ alkynyl or C₃-C₆ cycloalkyl each optionally substituted with one or more substituents selected from the group consisting of halogen, CN, NO₂, hydroxy, C₁-C₄ alkoxy, C₁-C₄ alkylthio, C₁-C₄ alkylsulfinyl and C₁-C₄ alkylsulfonyl; or

R² and R³ can be taken together with the nitrogen to which they are attached to form a ring containing 2 to 6 atoms of carbon and optionally one additional atom of nitrogen, sulfur or oxygen, said ring may be optionally substituted with 1 to 4 substituents selected from the group consisting of C₁-C₂ alkyl, halogen, CN, NO₂ and C₁-C₂ alkoxy; and

each R⁴ and each R⁵ is independently H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₂-C₄ alkoxycarbonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl, or C₃-C₆ trialkylsilyl; or

each R⁴ and each R⁵ is independently a phenyl, benzyl, phenoxy, 5- or 6-membered heteroaromatic ring or an aromatic 8-, 9- or 10-membered fused heterobicyclic ring system, each ring optionally substituted with one to three substituents independently selected from the group consisting of C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; or

(R⁵)₂ when attached to adjacent carbon atoms can be taken together as —OCF₂O—, —CF₂CF₂O— or —OCF₂CF₂O—.

This invention also pertains to a composition for controlling an invertebrate pest comprising a biologically effective amount of a compound of Formula I or Formula II and at least one additional component selected from the group consisting of surfactants, solid diluents and liquid diluents. This invention also pertains to a composition comprising a biologically effective amount of a compound of Formula I or Formula II and an effective amount of at least one additional biologically active compound or agent.

This invention also pertains to a method for controlling an invertebrate pest comprising contacting the invertebrate pest or its environment with a biologically effective amount of a compound of Formula I or Formula rT (e.g., as a composition described herein). This invention also relates to such a method wherein the invertebrate pest or its environment is contacted with composition comprising a biologically effective amount of a compound of Formula I or Formula II or with a composition comprising a compound of Formula I or Formula II and a biologically effective amount of at least one additional compound or agent for controlling invertebrate pests.

### DETAILS OF THE INVENTION

In the above recitations, the term “alkyl”, used either alone or in compound words such as “alkylthio” or “haloalkyl” includes straight-chain or branched alkyl, such as, methyl, ethyl, ni-propyl, i-propyl, or the different butyl, pentyl or hexyl isomers. “Alkenyl” includes straight-chain or branched alkenes such as 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as 1-propynyl, 2-propynyl and the different butynyl, pentynyl and hexynyl isomers. “Alkynyl” can also include moieties comprised of multiple triple bonds such as 2,5-hexadiynyl. “Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers. “Cycloalkyl” includes, for example, cyclopropyl, cyclobutyl, cyclopentyl and cyclohexyl. “Alkylamino”, “alkenylthio”, “alkenylsulfinyl”, “alkenylsulfonyl”, “alklynylsulfinyl”, “alkynylsulfonyl”, and the like, are defined analogously to the above examples. Examples of “alkoxycarbonyl” include CH₃OC(═O), CH₃CH₂OC(—O), CH₃CH₂CH₂OC(—O), (CH₃)₂CHOC(—O) and the different butoxy- or pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. The term “dialkylamino” includes amino functions substituted with two alkyl groups that may be the same or different. “Dialkylaminocarbonyl” is defined analogously, and examples include (CH₃)₂NC(═O) and CH₃CH₂NCH₃C(═O).

The term “heterocyclic ring” or “heterocyclic ring system” denotes rings or ring systems in which at least one ring atom is not carbon and comprises 1 to 4 heteroatoms independently selected from the group consisting of nitrogen, oxygen and sulfur, provided that each heterocyclic ring contains no more than 4 nitrogens, no more than 2 oxygens and no more than 2 sulfurs. The heterocyclic ring can be attached through any available carbon or nitrogen by replacement of hydrogen on said carbon or nitrogen. The term “aromatic ring system” denotes fully unsaturated carbocycles and heterocycles in which the polycyclic ring system is aromatic (where aromatic indicates that the Hückel rule is satisfied for the ring system). The term “heteroaromatic ring” denotes fully aromatic rings in which at least one ring atom is not carbon and comprises 1 to 4 heteroatoms independently selected from the group consisting of nitrogen, oxygen and sulfur, provided that each heterocyclic ring contains no more than 4 nitrogens, no more than 2 oxygens and no more than 2 sulfurs (where aromatic indicates that the Hückel rule is satisfied). The heterocyclic ring can be attached through any available carbon or nitrogen by replacement of hydrogen on said carbon or nitrogen. The term “aromatic heterocyclic ring system” includes fully aromatic heterocycles and heterocycles in which at least one ring of a polycyclic ring system is aromatic (where aromatic indicates that the Hückel rule is satisfied). The term “fused heterobicyclic ring system” includes a ring system comprised of two fused rings in which at least one ring atom is not carbon and can be aromatic or non aromatic, as defined above.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCR₂C≡CCH₂. Examples of “haloalkoxy” include CF₃O, CCl₃CH₂O, HCF₂CH₂CH₂O and CF₃CH₂O.

The total number of carbon atoms in a substituent group is indicated by the “Cᵢ-Cⱼ” prefix where i and j are numbers from 1 to 6. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of Formula I contains a heterocyclic ring, all substituents are attached to this ring through any available carbon or nitrogen by replacement of a hydrogen on said carbon or nitrogen.

When a group contains a substituent which can be hydrogen, for example R³, then, when this substituent is taken as hydrogen, it is recognized that this is equivalent to said group being unsubstituted. When the number of optional substituents on a group is 0, for example when n is 0, then it is recognized that this is equivalent to said group being unsubstituted. When a bond is depicted as floating, the substituent may be attached to any of the available carbons on the ring by replacement of hydrogen; for bicyclic ring systems, the substituent or substituents may be attached to either ring of the bicyclic ring system, or both rings. For example, when n is 1, a single R⁴ substituent may be attached to Formula I or Formula II at any of the indicated positions 2, 3, 4, 5, 6 or 7; and when n is 2, two independent R⁴ substituents may be attached at the 2,3; 2,4; 2,5; 2, 6 or 2,7 positions or any other combination of 2 positions. When R² and R³ are taken together with the nitrogen to which they are attached to form a ring, said ring can be optionally substituted on any available carbon or optionally nitrogen in said ring.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers, or as an optically active form.

The present invention comprises compounds selected from Formula I or Formula II, N-oxides and agriculturally suitable salts thereof. One skilled in the art will appreciate that not all nitrogen containing heterocycles can form N-oxides since the nitrogen requires an available lone pair for oxidation to the oxide; one skilled in the art will recognize those nitrogen containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-clhloroperbenzoic acid (MCPBA), hydrogen peroxide, alkyl hydroperoxides such as t-butyl hydroperoxide, sodium perborate, and dioxiranes such as dimethydioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Hetetrocyclic Chemistry, vol. 3, pp 18-19, A. J. Boulton and A. McKillop, Eds., Pergamon Press; M. R. Grimmett and B. R. T. Keene in Advances in Heterocyclic Chemistry, vol. 43, pp 139-151, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

The salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

Preferred for reasons including ease of synthesis and/or greater arthropodicidal efficacy are:

Preferred 1. Compounds of Formula I wherein

A and B are both O;

R¹ is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl; and

n is 0 to 2.

Preferred 2. Compounds of Formula II wherein

A and B are both O;

R¹ is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl; and

n is 0 to 2.

Preferred 3. Compounds of Preferred 1 or Preferred 2 wherein

J is a phenyl ring or a 5- or 6-membered heteroaromatic ring selected from the group consisting of J-1, J-2, J-3 and J-4, each J ring optionally substituted with 1 to 3 R⁵

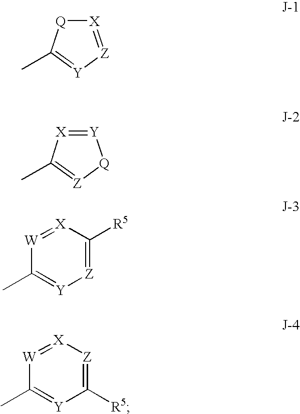

Q is O, S or NR⁵;

W, X, Y and Z are independently N or CR⁵, provided that in J-3 and J-4 at least one of W, X, Y or Z is N;

R² is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl;

R³ is C₁-C₆ alkyl, C₂-C₆ alkenyl, C₂-C₆ alkynyl or C₃-C₆ cycloalkyl each optionally substituted with one or more substituents selected from the group consisting of halogen, CN, C₁-C₂ alkoxy, C₁-C₂ alkylthio, C₁-C₂ alkylsulfinyl and C₁-C₂ alkylsulfonyl;

one R⁴ group is attached to the naphthyl ring system at the 2-position or 7-position, and said R⁴ is C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl or C₁-C₄ haloalkylsulfonyl;

each R⁵ is independently H, C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl, C₃-C₈ dialkylaminocarbonyl; or

each R⁵ is independently a phenyl, benzyl or a 5- or 6-membered heteroaromatic ring, each ring optionally substituted with C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; or

(R⁵)₂ when attached to adjacent carbon atoms can be taken together as —OCF₂O—, —CF₂CF₂O— or —OCF₂CF₂O—; and

n is 1 to 2.

Preferred 4. Compounds of Formula I of Preferred 3 wherein R² is H; R³ is C₁-C₄ alkyl; and at least one of the R⁵ substituents is ortho to the NR¹C(═B) moiety.

Preferred 5. Compounds of Preferred 4 wherein R³ is methyl.

Preferred 6. Compounds of Formula II of Preferred 3 wherein

R¹ is H or C₁-C₄ alkyl;

R² is H or C₁-C₄ alkyl;

R³ is C₁-C₄ alkyl optionally substituted with halogen, CN, OCH₃, or S(O)ₚCH₃;

one R⁵ group is attached to the J at the position ortho to the C(═B)NR¹ moiety, and said R⁵ is C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl; C₃-C₈ dialkylaminocarbonyl or a phenyl, benzyl, or a 5- or 6-membered heteroaromatic ring, each ring optionally substituted with halogen, CN, NO₂, C₁-C₄ alkyl, C₂-C₄ alkenyl, C₂-C₄ alkynyl, C₃-C₆ cycloalkyl, C₁-C₄ haloalkyl, C₁-C₄ alkoxy or C₁-C₄ haloalkoxy;

and a second optional R⁵ group is independently C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl; C₃-C₈ dialkylaminocarbonyl or a phenyl, benzyl, or a 5- or 6-membered heteroaromatic ring, each ring optionally substituted with halogen, CN, NO₂, C₁-C₄ alkyl, C₂-C₄ alkenyl, C₂-C₄ alkynyl, C₃-C₆ cycloalkyl, C₁-C₄ haloalkyl, C₁-C₄ alkoxy or C₁-C₄ haloalkoxy.

Preferred 7. Compounds of Preferred 6 wherein J is phenyl, pyrazole, pyrrole, pyridine or pyrimidine, each substituted with one R⁵ attached to the J at the position ortho to the C(═B)NR¹ moiety and a second optional R⁵.

Preferred 8. Compounds of Preferred 7 wherein

R¹ and R² are each H;

one R⁴ is attached at the 7-position ortho to the NR¹C(═X)J moiety and is selected from the group consisting of C₁-C₃ alkyl, CF₃, OCF₃, OCHF₂, S(O)ₚCF₃, S(O)ₚCHF₂ and halogen and an optional second R⁴ is attached at the 5-position pair to the NR¹C(═X)J moiety and is selected from the group consisting of halogen, C₁-C₃ alkyl and C₁-C₃ haloalkyl.

Preferred 9. Compounds of Preferred 8 wherein

J is J-1;

Q is NR^{5a};

X is N or CH;

Y is CH;

Z is CR^{5b};

R^{5a} is a phenyl or 2-pyridyl ring substituted with one or two substituents selected from the group consisting of halogen, C₁-C₄ alkyl, C₁-C₄ haloalkyl or C₁-C₄ haloalkoxy; and

R^{5b} is halogen or CF₃.

Specifically preferred compounds are those selected from the group consisting of

N-methyl —N′-(2-bromo-4-fluoropheny)-1,8-naphthalene-dicarboxamide,

N-methyl 8-[(3,4-difluorophenyl)carbonylamino]-1-naphthalenecarboxamide and

N-methyl 8-[(2-thienyl)carbonylamino]-1-naphthalenecarboxamide.

This invention also pertains to a composition for controlling invertebrates comprising a biologically effective amount of a compound of Formula I or Formula II and at least one additional component selected from the group consisting of surfactants, solid diluents and liquid diluents. This invention also pertains to a composition comprising a biologically effective amount of a compound of Formula I or Formula II and an effective amount of at least one additional biologically active compound or agent. Preferred compositions are those comprising compounds of Formula I or Formula II as preferred in Preferred 1 through 9, and the specifically preferred compounds above.

This invention also pertains to a method for controlling arthropods comprising contacting the arthropods or their environment with an arthropodicidally effective amount of a compound of Formula I or Formula II, and N-oxide or agriculturally suitable salts thereof (e.g. as a composition comprising a compound of Formula I or Formula II). This invention also relates to such a method wherein the invertebrate pest or its environment is contacted with a biologically effective amount of a compound of Formula I or Formula II or a composition comprising a compound of Formula I or Formula II and a biologically effective amount of at least one additional compound or agent for controlling invertebrate pests. Preferred methods are those comprising compounds of Formula I or Formula II as preferred in Preferred 1 through 9, and the specifically preferred compounds above.

Of note are certain compounds of Formula Ih and Formula IIc including all geometric and stereoisomers, and agriculturally suitable salts thereof

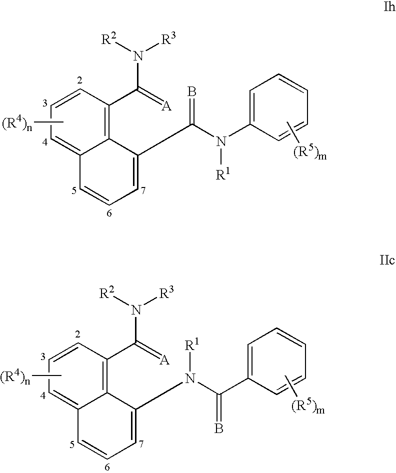

wherein:

A and B are independently O or S;

m is 1 to 5;

n is 0 to 4;

R¹ is H; or C₁-C₆ alkyl, C₂-C₆ alkenyl, C₂-C₆ alkynyl or C₃-C₆ cycloalkyl each optionally substituted with one or more substituents selected from the group consisting of halogen, CN, NO₂, hydroxy, C₁-C₄ alkoxy, C₁-C₄ alkylthio, C₁-C₄ alkylsulfinyl, C₁-C₄ alkylsulfonyl, C₂-C₄ alkoxycarbonyl, C₁-C₄ alkylamino, C₂-C₈ dialkylamino and C₃-C₆ cycloalkylamino; or

R¹ is C₂-C₆ alkylcarbonyl, C₂-C₆ alkoxycarbonyl, C₂-C₆ alkylaminocarbonyl or C₃-C₈ dialkylaminocarbonyl;

R² is H, C₁-C₆ alkyl, C₂-C₆ alkenyl, C₂-C₆ alkynyl, C₃-C₆ cycloalkyl, C₁-C₄ alkoxy, C₁-C₄ alkylamino, C₂-C₈ dialkylamino, C₃-C₆ cycloalkylamino, C₂-C₆ alkoxycarbonyl or C₂-C₆ alkylcarbonyl;

R³ is H; or C₁-C₆ alkyl, C₂-C₆ alkenyl, C₂-C₆ alkynyl or C₃-C₆ cycloalkyl each optionally substituted with one or more substituents selected from the group consisting of halogen, CN, NO₂, hydroxy, C₁-C₄ alkoxy, C₁-C₄ alkylthio, C₁-C₄ alkylsulfinyl and C₁-C₄ alkylsulfonyl; or

R² and R³ can be taken together with the nitrogen to which they are attached to form a ring containing 2 to 6 atoms of carbon and optionally one additional atom of nitrogen, sulfur or oxygen, said ring may be optionally substituted with 1 to 4 substituents selected from the group consisting of C₁-C₂ alkyl, halogen, CN, NO₂ and C₁-C₂ alkoxy; and

each R⁴ and each R⁵ is independently H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₂-C₄ alkoxycarbonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl, or C₃-C₆ trialkylsilyl; or

each R⁴ and each R⁵ is independently phenyl optionally substituted with C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl.

Also of note are arthropodicidal compositions comprising an arthropodicidally effective amount of a compound of Formula Ih or Formula IIc noted above and at least one additional component selected from the group consisting of surfactants, solid diluents or liquid diluents.

Also of note are methods for controlling arthropods comprising contacting the arthropods or their environment with an arthropodicidally effective amount of a compound of Formula Ih or Formula IIc noted above.

Selection 1. Of note are selected compounds of Formula Ih wherein

A and B are both O;

m is 1 to 3;

n is 0 to 2;

R¹ is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl; and

each R⁴ and each R⁵ is independently H, C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl.

Selection 2. Of note are selected compounds of Selection I wherein

R² is H;

R³ is C₁-C₄ alkyl;

each R⁴ and each R⁵ is independently H, C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl; and

at least one of the R⁵ substituents is ortho to the amide linkage.

Selection 3. Of note are selected compounds of Selection 2 wherein R³ is methyl.

Selection 4. Of note are selected compounds of Formula IIc wherein

A and B are both O;

m is 1 to 3;

n is 0 to 2;

R¹ is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl; and

each R⁴ and each R⁵ is independently H, C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl.

Selection 5. Of note are selected compounds of Selection 4 wherein

R² is H;

R³ is C₁-C₆ alkyl optionally substituted with C₁-C₄ alkoxy or C₁-C₄ alkylthio;

each R⁴ and each R⁵ is independently H, C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl; and

at least one of the R⁵ substituents is ortho to the amide linkage.

As noted above, each J is independently a phenyl ring, a naphthyl ring system, a 5- or 6-membered heteroaromatic ring or an aromatic 8-, 9- or 10-membered fused heterobicyclic ring system wherein each ring or ring system is optionally substituted with 1 to 5 R⁵. The term “optionally substituted” in connection with these J groups refers to groups which are unsubstituted or have at least one non-hydrogen substituent that does not extinguish the arthropodicidal activity possessed by the unsubstituted analog. An example of phenyl optionally substituted with 1 to 5 R⁵ is the ring illustrated as U-1 in Exhibit 1, wherein R^{v} is R⁵ and r is an integer from 1 to 5. An example of a naphthyl group optionally substituted with 1 to 5 R⁵ is illustrated as U-85 in Exhibit 1, wherein R^{v} is R⁵ and r is an integer from 1 to 5. Examples of 5- or 6-membered heteroaromatic rings optionally substituted with 1 to 5 R⁵ include the rings U-2 through U-53 illustrated in Exhibit 1 wherein R^{v} is R⁵ and r is an integer from 1 to 5. Note that J-1 through J-4 below also denote 5- or 6-membered heteroaromatic rings. Note that U-2 through U-20 are examples of J-1, U-2 1 through U-35 and U-40 are examples of J-2, U-41 through U-48 are examples of J-3 and U-49 through U-53 are examples of J-4. Examples of aromatic 8-, 9- or 10-membered fused heterobicyclic ring systems optionally substituted with 1 to 4 R³ include U-54 through U-84 illustrated in Exhibit 1 wherein R^{v} is R⁵ and r is an integer from 1 to 5.

Although R^{v} groups are shown in the structures U-I through U-85, it is noted that they do not need to be present since they are optional substituents. Note that when R^{v} is H when attached to an atom, this is the same as if said atom is unsubstituted. The nitrogen atoms that require substitution to fill their valence are substituted with H or R^{v}. Note that some U groups can only be substituted with less than 5 R^{v} groups (e.g. U-14, U-1 5, U-18 through U-21 and U-32 through U-34 can only be substituted with one R^{v}). Note that when the attachment point between (R^{v})ᵣ and the U group is illustrated as floating, (R^{v})ᵣ can be attached to any available carbon atom of the U group. Note that when the attachment point on the U group is illustrated as floating, the U group can be attached to the remainder of Formula I through any available carbon of the U group by replacement of a hydrogen atom.

### Exhibit 1

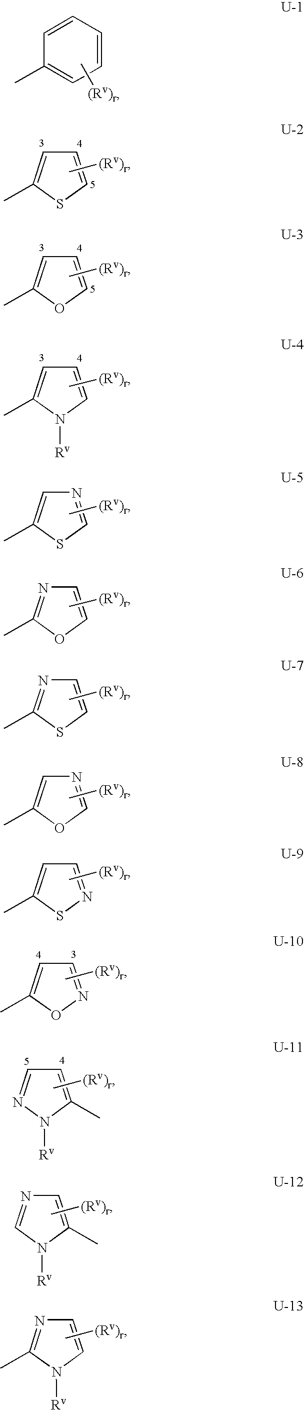

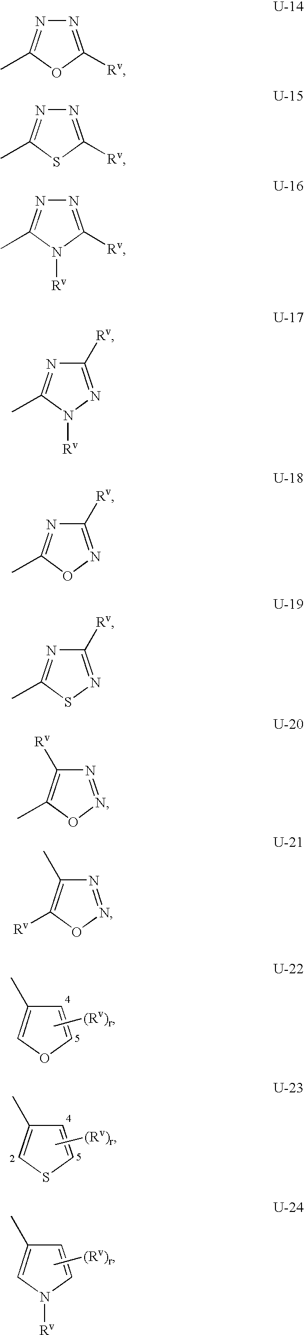

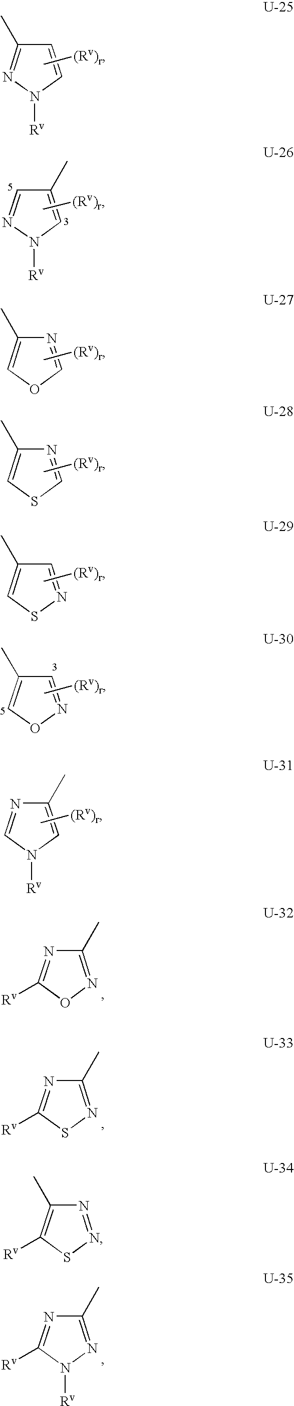

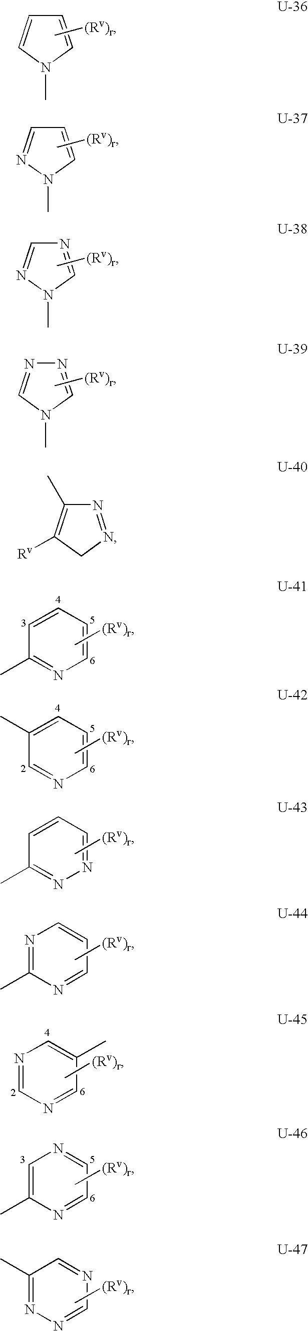

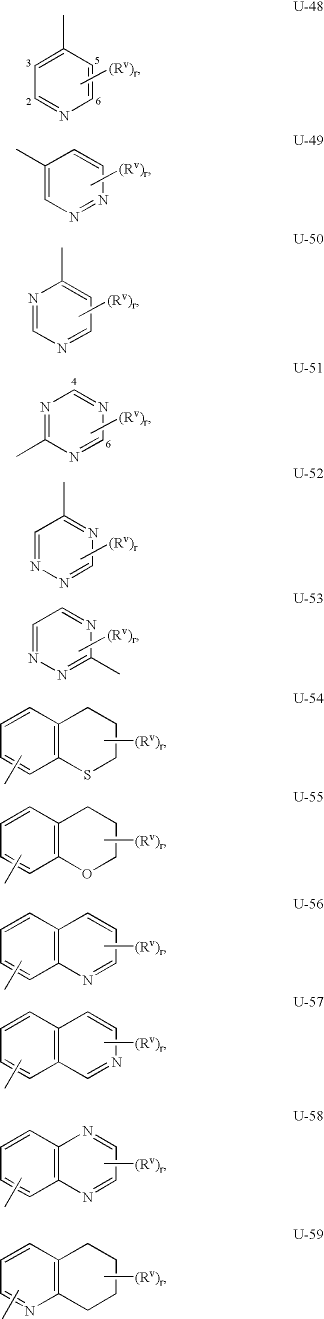

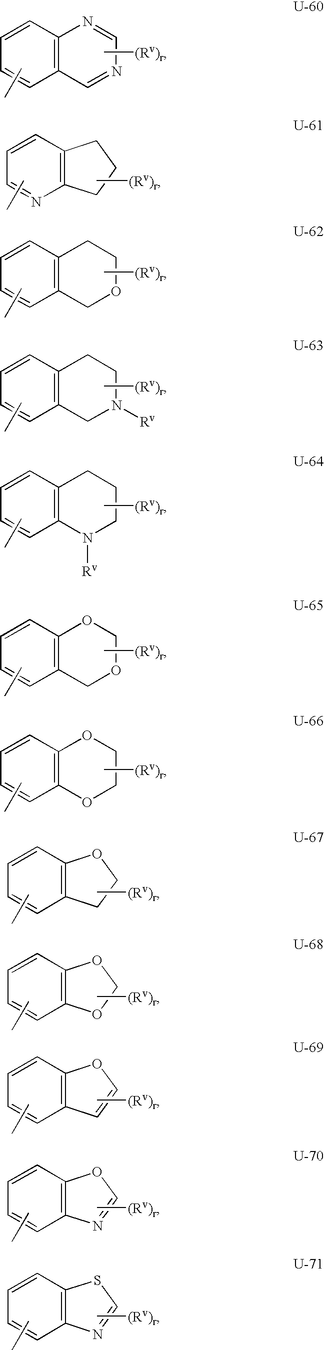

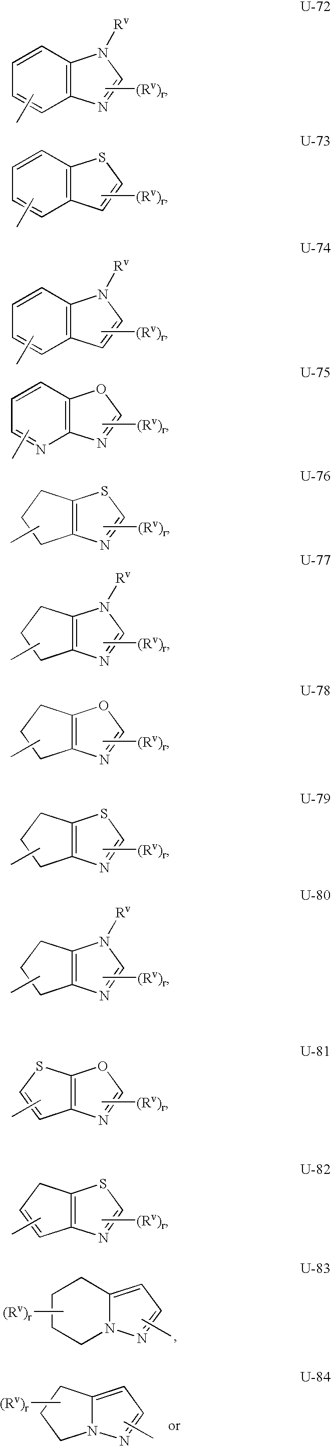

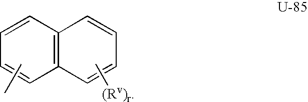

The compounds of Formula I and Formula II can be prepared by one or more of the following methods and variations as described in Schemes 1-2 1. The definitions of R¹, R², R³, R⁴, A, B, m and n in the compounds described in the Schemes below are as defined above in the Summary of the Invention or their subsets.

Compounds of Formula I can be prepared by procedures outlined in Schemes 1-9. Compounds of Formulae Ia-h are various subsets of the compounds of Formula I.

Typical procedures are described in Scheme 1 and involve either coupling of a isonaphthalimide of Formula 2 with an amine of Formula 3 or coupling of a compound of Formula 4 with an amine of Formula 5 with or without the presence of a base. Typical bases include amine bases such as triethylamine, diisopropylethylamine and pyridine. In certain instances it is useful to use polymer supported acid scavengers such as polymer-bound diisopropylethylamine and polymer-bound dimethylaminopyridine. The amines of Formula 3 and Formula 5 are either commercially available, well represented in the chemical literature, or readily available from established literature procedures. Amides of Formula Ia can be converted to thioamides of Formula Ib using a variety of standard thio transfer reagents including phosphorus pentasulfide and Lawesson's reagent.

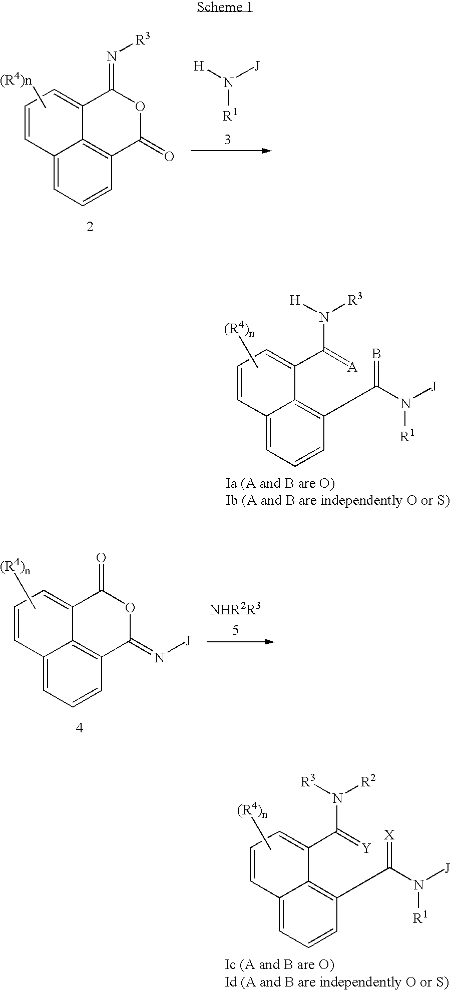

Compounds of Formula 2 and Formula 4 are typically prepared by coupling of a 1,8-naphthaloyl chloride of the Formula 6 with an amine of Formula 7 or an amine of Formula 8 respectively (Scheme 2). Typical acid scavengers include amine bases such as triethylamine, diisopropylethylamine and pyridine. In certain instances it is useful to use polymer supported acid scavengers such as polymer-bound diisopropylethylamine and polymer-bound dimethylaminopyridine. In some instances, the amine of Formula 7 can serve as the acid scavenger when used in excess. The compounds of 1,8-naphthaloyl chloride of the Formula 6 are well represented in the chemical literature and are typically prepared from the corresponding 1,8-naphthalic acids or the anhydrides with the use of chlorinating reagents commonly used for the transformation of carboxylic acid to carboxylic acid chloride. These commonly used chlorinating reagents include phosphorous oxychloride and phosphorous pentachloride.

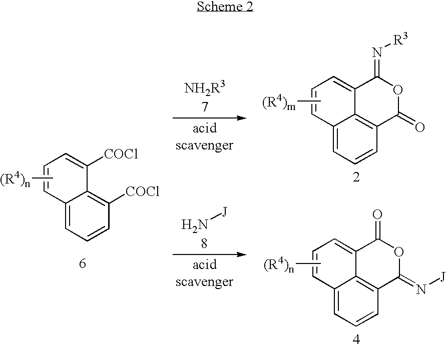

An alternate procedure for the preparation of compounds of Formula 2 and Formula 4 involves the cyclization of compounds of 8-aminocarbonyl-1-naphthalenecarboxylic acid of Formula 9 and Formula 10 respectively (Scheme 3) with the use of a dehydration reagent in an inert solvent at a temperature in the range of-30 to 30° C., with or without the presence of an acid scavenger. Typical dehydration reagents include dicyclohexylcarbodiimide and trifluoroacetic anhydride. Polymer supported reagents such as polymer-bound cyclohexylcarboduimide are useful. Typical acid scavengers include amine bases such as triethylamine, diisopropylethylamine and pyridine. In certain instances it is useful to use polymer supported acid scavengers such as polymer-bound diisopropylethylamine and polymer-bound dimethylaminopyridine. Typical inert solvents include aprotic solvents such as dichloromethane and 1-chlorobutane. A typical range of reaction temperature is from −5 to 25° C.

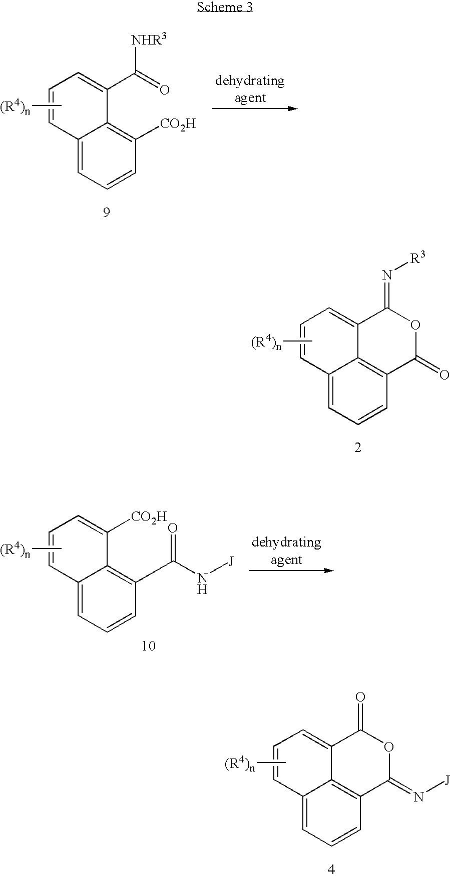

The preparation of 8-aminocarbonyl-1-naphthalenecarboxylic acids of Formula 9 and Formula 10 involves the coupling of 1,8-naphthalic anhydrides of Formula 11 with amines of Formula 7 or amines of Formula 8 respectively (Scheme 4) in an inert solvent at a temperature in the range of 0 to 30° C. A typical inert solvent is dimethylformamide. The reaction is commonly conducted in the temperature range of 5 to 10° C.

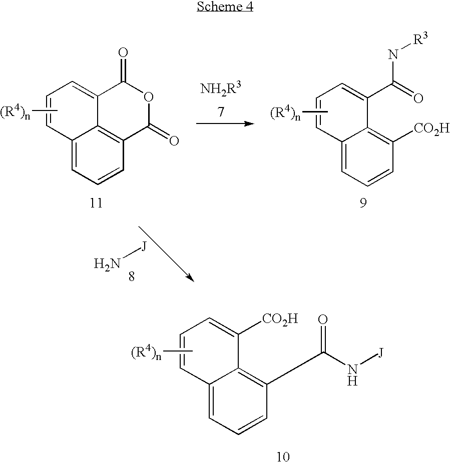

Alternate procedures for the preparation of compounds of 8-aminocarbonyl-1-naphthalenecarboxylic acid of Formula 9 and Formula 10 involve the hydrolysis of 10 compounds of 1,8-naphthalimide of Formula 12 and Formula 13 respectively (Scheme 5) using a hydroxide such as sodium hydroxide or potassium hydroxide in a protic solvent system such as water-methanol or water-dioxane at elevated temperature. The reaction is usually conducted at the reflux temperature of the reaction mixture. The compounds of 1,8-naphthalimide of Formula 12 and Formula 13 are typically prepared by condensing a 1,8-naphthalic anhydride of Formula II with an amine of Formula 7 or Formula 8 respectively at an elevated temperature, usually in the presence of an acid. A typical reaction involves refluxing the 1,8-naphthalic anhydride of Formula 11 and the amine of Formula 7 or Formula 8 in acetic acid.

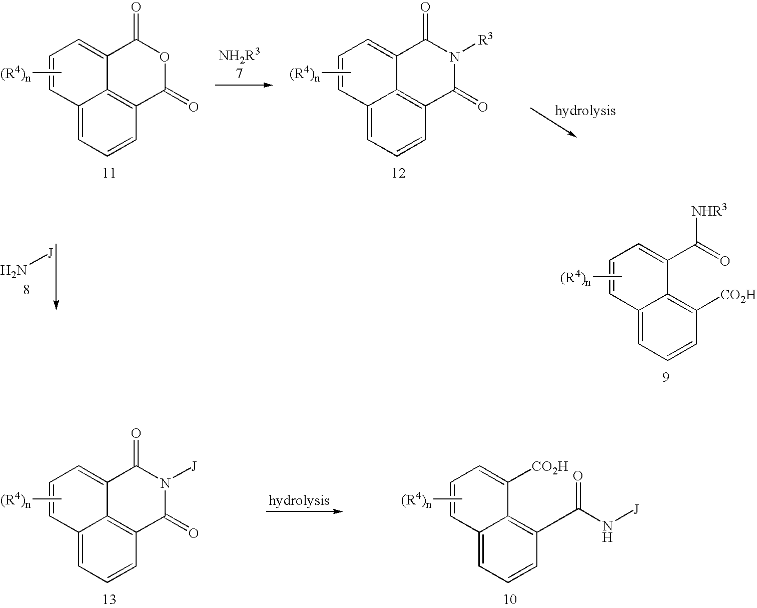

An alternate procedure for the preparation of compounds of Formula I involves the coupling of 8-aminocarbonyl-1-naphthoyl chlorides of Formula 14 and Formula 15 with an amine of Formula 3 or an amine of Formula 5 respectively (Scheme 6), in the presence of an acid scavenger. Typical acid scavengers include amine bases such as triethylamine, diisopropylethylamine and pyridine. In certain instances it is useful to use polymer supported acid scavengers such as polymer-bound diisopropylethylamine and polymer-bound dimethylaminopyridine. Typically, the reaction temperature range is 0 to 25° C.

8-Aminocarbonyl-1-naphthoyl chlorides of Formula 14 and Formula 15 are available from coupling the corresponding 1,8-naphthaloyl dichloride of Formula 6 with an amine of Formula 5 or Formula 3 respectively in the presence of an acid scavenger (Scheme 7). Typical acid scavengers include amine bases such as triethylamine, diisopropylethylamine and pyridine. In certain instances it is useful to use polymer supported acid scavengers such as polymer-bound diisopropylethylamine and polymer-bound dimethylaminopyridine.

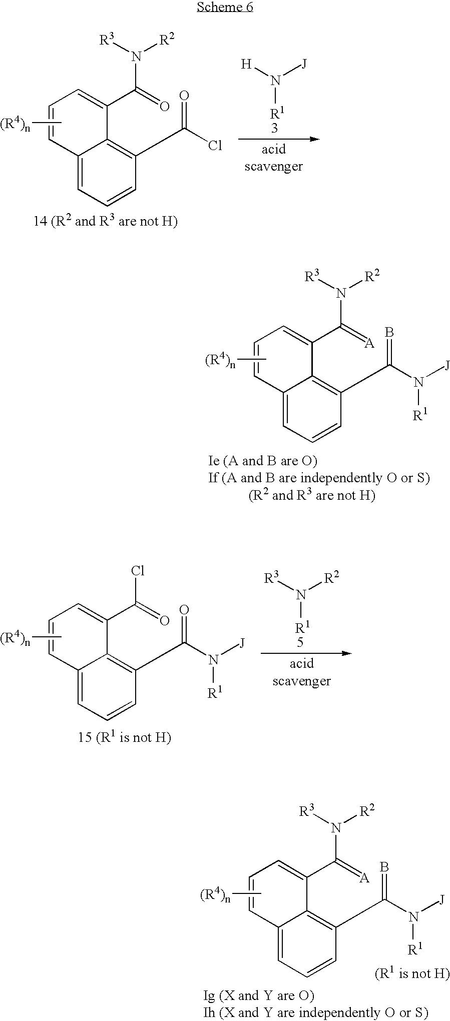

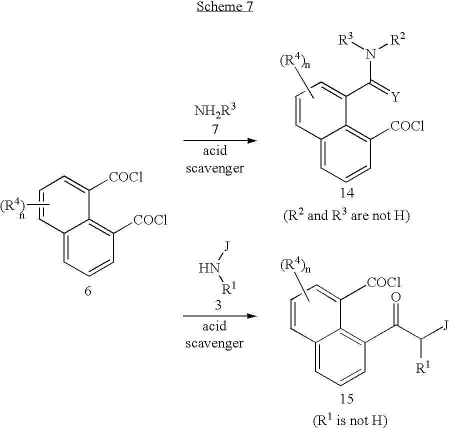

Another procedure for the preparation of compounds of Formula I involves the stepwise carbamoylation or thiocarbamoylation of a naphthalene of Formula 20 (Schemes 8 and 9). Upon treatment with a Lewis acid under Friedel-Crafts reaction conditions, compounds of 1-naphthalenecarboxamide or 1-naphthalenethiocarboxamide of Formula 16 or Formula 17 couple with a carbamoyl chloride or thiocarbamoyl chloride of Formula 18 or Formula 19 to provide a compound of Formula I (Scheme 8). Commonly used Lewis acids include aluminum chloride and stannic chloride.

The preparation of compounds of 1-naphthalenecarboxamides or 1-naphthalenethiocarboxamides of Formula 16 or Formula 17 (Scheme 9) involves the coupling of a naphthalene of Formula 20 and a carbamoyl chloride or thiocarbamoyl chloride of Formula 19 or Formula 18 respectively in the presence of a Lewis acid such as aluminum chloride and stannic chloride under Friedel-Crafts reaction conditions. The naphthalenes of Formula 20, the carbamoyl chlorides and the thiocarbamoyl chlorides of Formula 18 and Formula 19 are commercially available, well described in the chemical literature, or can be prepared following established literature procedures.

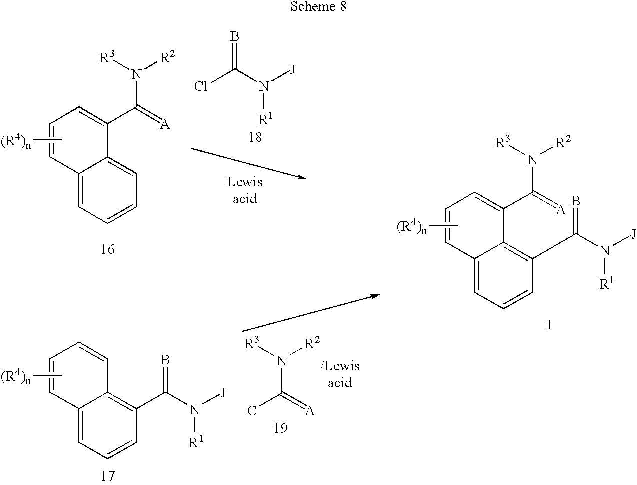

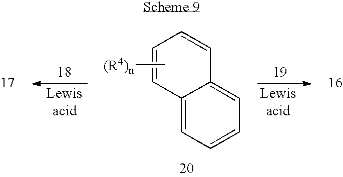

The compounds of Formula II can be prepared by one or more of the following methods and variations as described in Schemes 10-13. Compounds of Formulae IIa-b are subsets of the compounds of Formula II.

A typical procedure is described in Scheme 10 and involves coupling of an 8-amino-naphthalene-1-carboxamide of Formula 21 with a carbonyl chloride of Formula 22 in the presence of an acid scavenger to provide the compounds of Formula IIa, or with thiocarbonyl chloride of Formula 22 in the presence of an areneselenolate to provide the compounds of Formula IIb. Typical acid scavengers include amine bases such as triethylamine, diisopropylethylamine and pyridine, other scavengers include hydroxides such as sodium and potassium hydroxide and carbonates such as sodium carbonate and potassium carbonate. In certain instances it is useful to use polymer supported acid scavengers such as polymer-bound diisopropylethylamine and polymer-bound dimethylaminopyridine. Typical areneselenolates include sodium phenylselenolate. The carbonyl chlorides and the thiocarbonyl chlorides of Formula 22 are commercially available, well represented in the chemical literature, or readily prepared according to established literature procedures. Amides of Formula IIa can be converted to thioamides of Formula IIb using standard thio transfer reagents such as phosphorus pentasulfide and Lawesson's reagent.

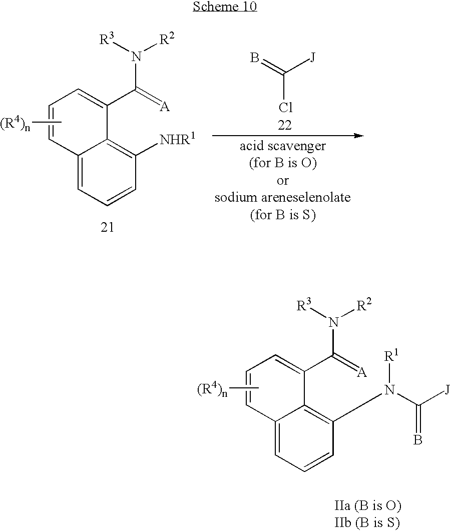

Another procedure for the preparation of compounds of Formula IIa involves coupling of an 1-amino-naphthalene-8-carboxamide or 1-amino-naphthalene-8-thiocarboxamide of Formula 21 with a carboxylic acid of Formula 23 in the presence of a dehydration reagent such as dicyclohexylcarbodiimide (DCC). Polymer supported reagents such as polymer-bound cyclohexylcarboduimide are useful. Benzoic acids of Formula 23 are commercially available, well represented in the chemical literature, or readily prepared according to established literature procedures. The procedures of Scheme 10 and Scheme 11 are only representative examples of useful methods for the preparation of Formula II compounds as the literature is extensive for the preparation of carboxamides.

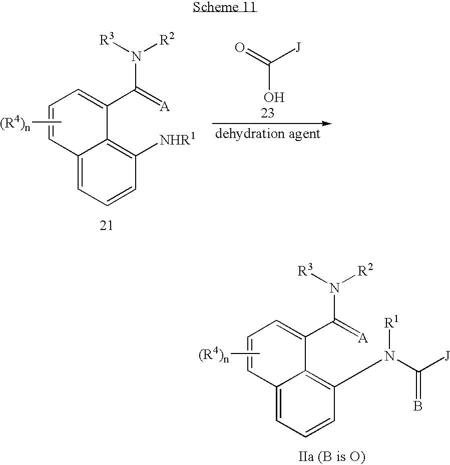

8-Amino-naphthalene-1-carboxamides and 8-amino-naphthalene-1-thiocarboxamides of Formula 21a are typically available from the corresponding 8-nitro-naphthalene-1-carboxamides and 8-nitro-naphthalene-1-thiocarboxamides of Formula 24 respectively via reduction of the nitro group. Typical procedures involve reduction with hydrogen in the presence of a metal catalyst such as palladium on carbon or platinum oxide and in hydroxylic solvents such as ethanol and isopropanol. These procedures are well documented in the chemical literature. R¹ substituents such as alkyl, substituted alkyl and the like can generally be introduced at this stage through known procedures including either direct alkylation or through the generally preferred method of reductive alkylation of the amine. A commonly employed procedure is to combine the aminonaphthalene 21a with an aldehyde in the presence of a reducing agent such as sodium cyanoborohydride to produce the compounds of Formula 21 where R¹ is other than H.

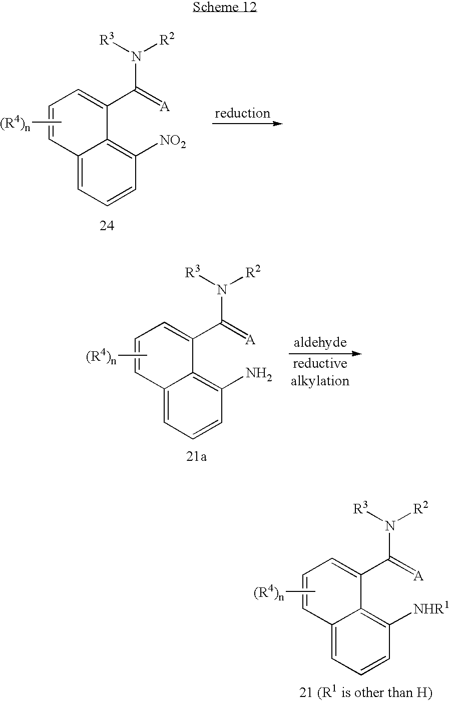

The intermediate amides of Formula 24 are readily prepared from 8-nitro-naphthalene-1-carboxylic acids that are commercially available, known in the literature, or can be prepared by established literature procedures for the derivatization of naphthalenes or aromatic groups in general. Typical methods for amide formation can be applied here. These include direct dehydrative coupling of acids of Formula 25 with amines of Formula 5 using for example DCC, and conversion of the acids to an activated form such as the acid chlorides or anhydrides and subsequent coupling with amines to form amides of formula 24a. Also, diethyl cyanophosphonate is a useful reagent for this type of reaction involving activation of the acid. The chemical literature is extensive on this type of reaction. Amides of Formula 24a are readily converted to thioamides of Formula 24b by using commercially available thio transfer reagents such as phosphorus pentasulfide and Lawesson's reagent.

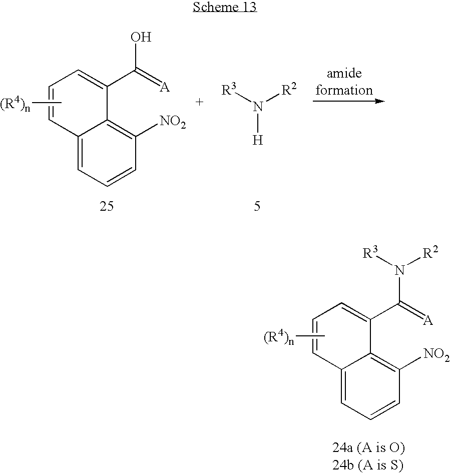

Benzoic acids of Formula 23a, (compounds of Formula 23 wherein J is an optionally substituted phenyl ring) are well known in the art. Preparation of certain heterocyclic acids of Formula 4 are described in Schemes 14-21. A variety of heterocyclic acids and general methods for their synthesis may be found in World Patent Application WO 98/57397.

The synthesis of representative pyridine acids (23b) is depicted in Scheme 14. This procedure involves the known synthesis of pyridines from β-ketoesters and 4-aminobutenones (29). Substituent groups R⁵(c) and R⁵(d) include e.g. alkyl and haloalkyl.

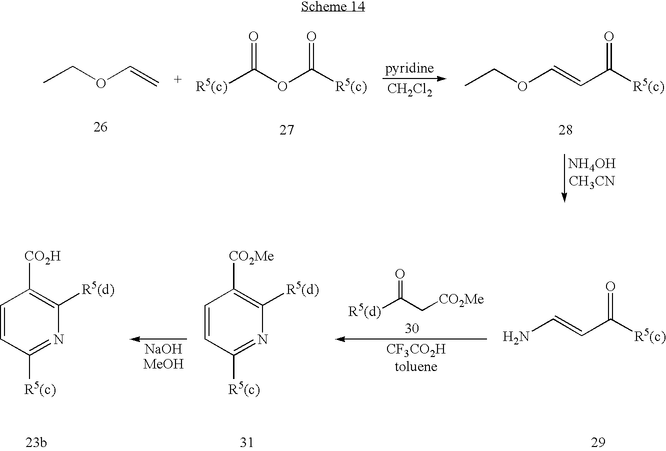

The synthesis of representative pyrimidine acids (23c) is depicted in Scheme 15. This procedure involves the known synthesis of pyrimidines from vinylidene-β-ketoesters (33) and amidines (34). Substituent groups R⁵(c) and R⁵(d) include e.g. alkyl and haloalkyl.

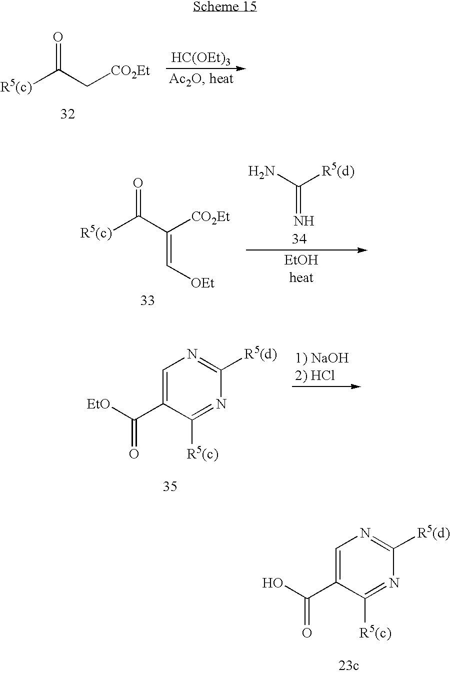

Syntheses of representative pyrazole acids (23d) are depicted in Schemes 16-21. The synthesis of 23d in Scheme 16 involves as the key step introduction of the R⁵(c) substituent via arylation or alkylation of the pyrazole. The arylating or alkylating agent R⁵(c)-Lg (wherein Lg is a leaving group such as Cl, Br, I, sulfonates such as p-toluenesulfonate or methanesulfonate or sulfates such as —SO₂OR⁵(c)) includes R⁵(c) groups such as C₁-C₆ alkyl, C₂-C₆ alkenyl, C₂-C₆ alkynyl, C₂-C₆ alkylcarbonyl, C₂-C₆ alkoxycarbonyl, C₃-C₈ dialkylaminocarbonyl, C₃-C₆ trialkylsilyl; or phenyl, benzyl, benzoyl, 5- or 6-membered heteroaromatic ring each ring or ring system optionally substituted. Oxidation of the methyl group affords the pyrazole carboxylic acid. Some of the more preferred R⁵(d) groups include haloalkyl.

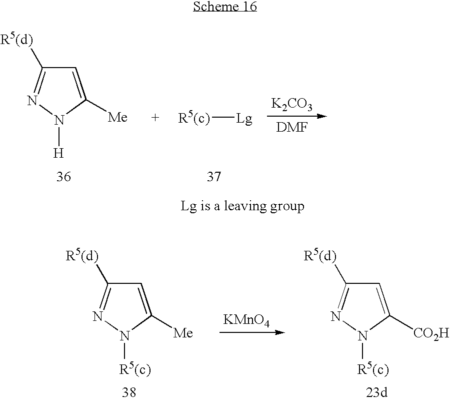

Synthesis of pyrazoles of Formula 23d are described in Scheme 17. These acids may be prepared via metallation of compounds of Formula 40 with lithium diisopropylamide (LDA) followed by quenching of the lithium salt with carbon dioxide affords metallation using lithium diisoprylamide (LDA) and carboxylation of compounds of Formula 40 as the key step. The R⁵(c) group is introduced in a manner similar to that of Scheme 16, i.e. via alkylation or arylation with a compound of Formula 37. Representative R⁵(d) groups include e.g. cyano, haloalkyl and halogen.

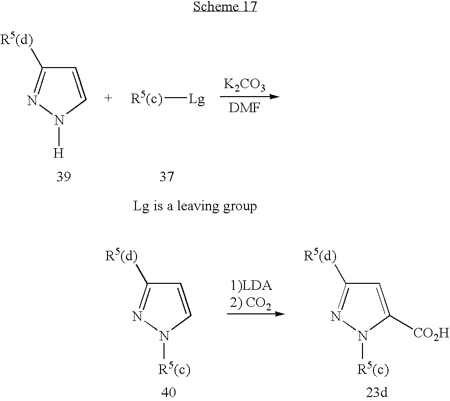

This procedure is particularly useful for preparing 1-(2-pyridinyl)pyrazolecarboxylic acids of Formula 23e as shown in Scheme 18. Reaction of a pyrazole of Formula 39 with a 2,3-dihalopyridine of Formula 37a affords good yields of the l-pyridylpyrazole of Formula 40a with good specificity for the desired regiochemistry. Metallation and carboxylation of compounds of Formula 40a as described above affords the 1-(2-pyridinyl)pyrazolecarboxylic acid of Formula 23e.

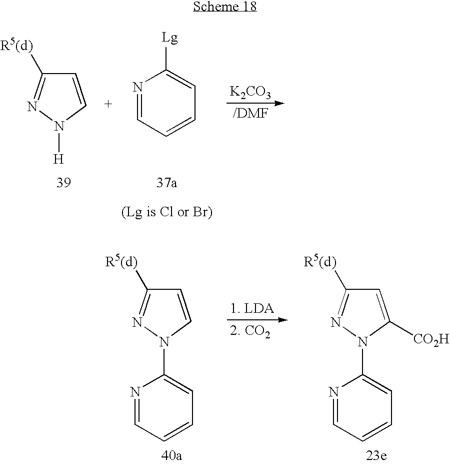

The synthesis of pyrazoles of Formula 4c is described in Scheme 19. They can be prepared via reaction of an optionally substituted phenyl hydrazine of Formula 41 with a ketopyruvate of Formula 42 to yield pyrazole esters of Formula 43. Hydrolysis of the esters affords the pyrazole acids of Formula 23d. This procedure is particularly useful for the preparation of compounds in which R⁵(c) is optionally substituted phenyl and R⁵(d) is haloalkyl.

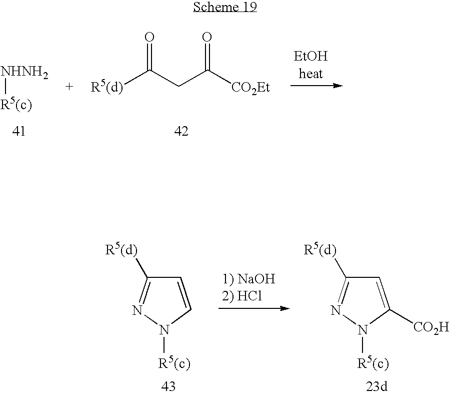

An alternate synthesis of pyrazole acids of Formula 23d is described in Scheme 20. They can be prepared via 3+2 cycloaddition of an appropriately substituted nitrilimine with either substituted propiolates of Formula 45 or acrylates of Formula 47. Cycloaddition with an acrylate requires additional oxidation of the intermediate pyrazoline to the pyrazole. Hydrolysis of the esters affords the pyrazole acids of Formula 23d. Preferred iminohalides for this reaction include the trifluoromethyl iminochloride of Formula 48 and the iminodibromide of Formula 49. Compounds such as 48 are known (J. Heterocycl. Chem. 1985, 22(2), 565-8). Compounds such as 49 are available by known methods (Tetrahedron Letters 1999, 40, 2605). These procedures are particularly useful for the preparation of compounds where R⁵(c) is optionally substituted phenyl and R⁵(d) is haloalkyl or bromo.

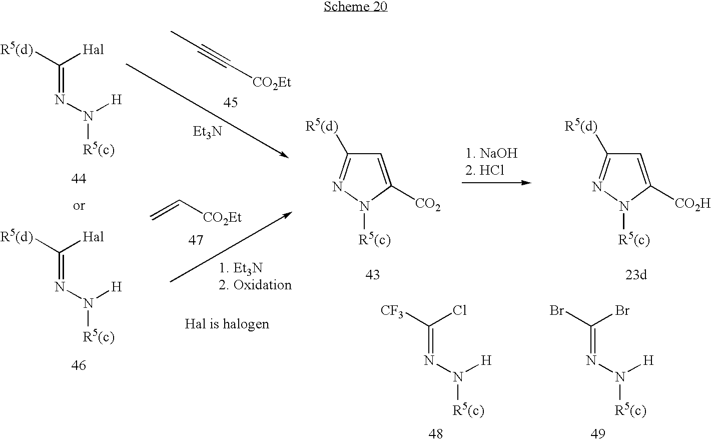

The starting pyrazoles of Formula 39 are known compounds. The pyrazole of Formula 39a (the compound of Formula 39 wherein R⁵(d) is CF₃) is commercially available. The pyrazoles of Formula 39c (compounds of Formula 39 wherein R⁵(d) is Cl or Br) can be prepared by literature procedures (Chem. Ber. 1966, 99(10), 33507). A useful alternative method for the preparation of compound 39c is depicted in Scheme 21. Metallation of the sulfamoyl pyrazole of Formula 50 with n-butyllithium followed by direct halogenation of the anion with either hexachloroethane (for R⁵(d) being Cl) or 1,2-dibromotetrachloroethane (for R⁵(d) being Br) affords the halogenated derivatives of Formula 51. Removal of the sulfamoyl group with trifluoroacetic acid (TFA) at room temperature proceeds cleanly and in good yield to afford the pyrazoles of Formula 39c. One skilled in the art will recognize that Formula 39c is a tautomer of Formula 39b.

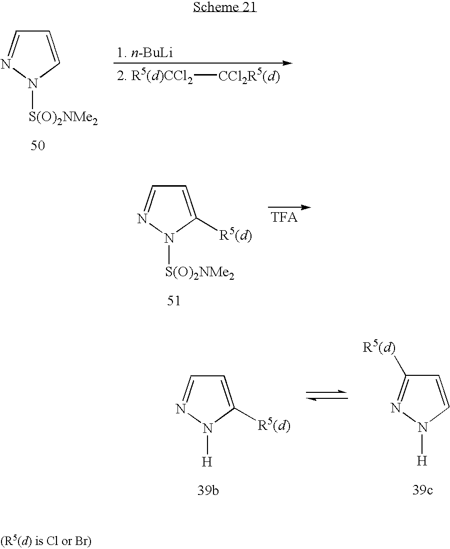

It is recognized that some reagents and reaction conditions described above for preparing compounds of Formula I and Formula II may not be compatible with certain functional groups present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula I and II. One skilled in the art will also recognize that it may be necessary to perform a combination of the steps illustrated in the above schemes in an order other than that implied by the particular sequence presented to prepare the compounds of Formula I and II.

One skilled in the art will also recognize that compounds of Formula I and II and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. ¹H NMR spectra are reported in ppm downfield from tetramethylsilane: s is singlet, d is doublet, t is triplet, q is quartet, m is multiplet, dd is doublet of doublets, dt is doublet of triplets, br s is broad singlet. m.p. is melting point.

### EXAMPLE 1

### Preparation of N-methyl-N′-(2-bromo-4-fluoropheny)-1,8-naphthalene-dicarboxamide

Step A: Preparation of 8-[(methylamino)carbonyl]-1-naphthalenecarboxylic Acid

To a stirred solution of 1,8-naphthalic anhydride (5 g, 25.3 mmole) in N,N-dimethylformarnmide (50 mL) at room temperature, methylamine (0.82 g, 26.5 mmole) in N,N-dimethylformamide (5 mL) was added. The reaction mixture was stirred for 2.5 hours. The solvent was removed under reduced pressure. The crude solid thus obtained was washed with a small amount of diethyl ether and air dried to give the product (5 g), containing 8-[(methylamino)carbonyl]-1-naphthalenecarboxylic acid and 1,8-naphthalic anhydride in a ratio of ˜7 to 1 determined by NMR analysis. This material was used for the subsequent reaction without further purification.

¹H NMR (DMSO-d6) δ: 2.77 (doublet, 3H), 7.55-7.65 (multiplet, 3H), 7.83 (doublet, 1H), 8.03-8.12 (multiplet, 2H), 8.39 (quartet, 1H).

Step B: Preparation of N-methyl —N′-(2-bromo-4-fluoropheny)-1,8-naphthalene-dicarboxamide

To a stirred solution of 8-[(methylamino)carbonyl]-1-naphthalenecarboxylic acid (22.3 g, 97.4 mmole) prepared as described in Step A and pyridine (15.4 g, 0.195 mole) in dichloromethane (300 mL) cooled to −5° C. under nitrogen atmosphere, trifluroacetic anhydride (22.5 g, 0.107 mole) diluted in dichloromethane (50 mL) was added in a dropwise manner. After the addition, the reaction mixture was further stirred at ambient temperature for one half of an hour. A small amount of solid was filtered off. The filtrate was concentrated under reduced pressure to a solid that was extracted with hot hexane (3×250 ml). The hexane extracts were combined and concentrated under reduced pressure to give a crude product of 3-(methylimino)-1H,3H-naphtho[1,8-cd]pyran-1-one (15 g).

The 3-(methylimino)-1H,3H-naphtho[1,8-cd]pyran-1-one (0.25 g, 1.19 mmole) thus obtained was dissolved in dichloromethiane (3 ml) and 2-bromo-4-fluoroaniline (0.17 g, 1.45 mmole) was added. The reaction mixture was stirred at room temperature for 48 hours. The solid was filtered, collected and washed with a small amount of methanol in dichloromethane and air dried to give N-methyl N′-(2-methyl-4-fluoropheny)-1,8-naphthalene-dicarboxamide (0.12 g, m.p. 249° C.).

¹H NMR (DMSO-d₆) δ: 2.37 (singlet, 3H), 2.72 (doublet, 3H), 7.05-7.12 (multiplet, 2H), 7.54-7.64 (multiplet, 3H), 7.75-7.83 (multiplet, 2H), 8.05-8.10 (multiplet, 2H), 8.33 (quartet, 1H.), 9.84 (singlet, 1H).

### EXAMPLE 2

### Preparation of N-methyl 8-[(3,4-difluorophenyl)carbonylamino]-1-naphthalenecarboxamide

Step A: Preparation of N-methyl 8-nitro-1-naphthalenecarboxamide

To a stirred solution of 8-nitro-1-naphthalenecarboxylic acid (1.5 g, 6.9 mmole), methylamine (0.22 g, 7.3 mmole), and imidazole (0.68 g, 8.3 mmole) in N,N-dimethylformamide (12 mL) at room temperature, diethyl cyanophosphonate (1.24 g, 7.6 mmole) was added. The reaction mixture was stirred overnight, poured into water (100 mL) and extracted with ethyl acetate (3×100 mL). The ethyl acetate extracts were combined, dried over anhydrous magnesium sulfate, filtered, and concentrated under reduced pressure to give a solid which was washed with a small amount of methanol to give the product (0.64 g) which was used without further purification.

¹H NMR (DMSO-d₆) δ: 2.76 (doublet, 3H), 7.72-8.37 (multiplet, 6H), 8.66 (broad quartet, 1H).

Step B: Preparation of N-methyl 8-amino-1-naththalenecarboxamide

N-methyl 8-nitro-1-naphthalenecarboxamide (0.5 g, 2.17 mmole) in methanol (5 mL) and dichlomethane (15 mL) was hydrogenated with a catalytic amount of palladium (10% on charcoal) under 30 psi hydrogen pressure for 1 hour. The reaction mixture was suction filtered through celite and concentrated under reduced pressure to provide the product (0.42 g) which was used without further purification.

¹H NMR (DMSO-d6) δ: 2.82 (doublet, 3H), 5.46 (singlet, 2H), 6.81-7.85 (multiplet, 6H), 8.59 (broad quartet, 1H).

Step C: Preparation of N-methyl 8-[(3,4-difluorophenyl)carbonylamino]-1-naphthalenecarboxamide

To a stirred solution of N-methyl 8-amino-1-naphthalenecarboxamide (0.2 g, 1 mmole) and pyridine (0.24 g, 3 mmole) in dichloromethane (10 mL), actyl chloride (94.2 mg, 1.2 mmole) in dichloromethane (2 mL) was added in portions. The mixture was stirred for 3 hours. The solid was collected with suction filtration (110 mg, m.p. >250° C.).

¹H NMR (DMSO-d6) δ: 2.43 (doublet, 3H), 7.46-8.10 (multiplet, 9H), 8.51 (broad quartet, 1H), 10.13 (singlet, 1H).

### EXAMPLE 3

### Preparation of N-methyl 8-[(2-thienyl)carbonylamino]-1-naphthalenecarboxamide

To a stirred solution of N-methyl 8-amino-1-naphthalenecarboxamide (0.2 g, 1 mmole) and pyridine (0.24 g, 3 mmole) in dichloromethane (10 mL), 2-thiophenecarboxylic acid chloride (176 mg, 1.2 mmole) in dichloromethane (2 mL) was added in portions. The mixture was stirred for 3 hours. The reaction mixture was washed sequentially with dilute hydrochloric acid, saturated sodium bicarbonate aqueous solution, and water. The solvent was partially removed under reduced pressure. The solid thus formed was collected with suction filtration (36 mg, m.p. 210° C.). ¹H NMR (DMSO-d6) δ: 2.53 (doublet, 3H), 7.26-8.10 (multiplet, 9H), 8.66 (broad quartet, 1H), 10.07 (singlet, 1H).

By the procedures described herein together with methods known in the art, the following compounds of Tables 1 to 10 can be prepared. The following abbreviations are used in the Tables: 1 is tertiary, s is secondary, z is normal, i is iso, c is cyclo, Me is methyl, Et is ethyl, Pr is propyl, i-Pr is isopropyl, t-Bu is tert butyl, Ph is phenyl, OMe is methoxy, OEt is ethoxy, SMe is methylthio, SEt is ethylthio, CN is cyano, NO₂ is nitro, TMS is trimethylsilyl, S(O)Me is methylsulfinyl, and S(O)₂Me is methylsulfonyl.

TABLE 1

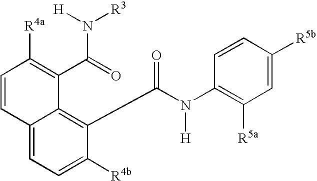

| R³ | R^{4a} | R^{4b} | R^{5a} | R^{5b} |
|---|---|---|---|---|
| Me | H | Me | H | CF₃ |
| Me | H | Me | H | OCF₃ |
| Et | H | Me | H | OCF₃ |
| Me | H | Me | Me | Br |
| Me | H | Me | Et | Br |
| Me | H | Me | Me | Cl |
| Me | H | Me | Et | Cl |
| Me | H | Me | Me | I |
| Me | H | Me | Me | CF₃ |
| Me | H | Me | Me | OCF₃ |
| Et | H | Me | Me | CF₃ |
| Me | H | Me | Me | SCF₃ |
| Me | H | Me | Me | SCHF₂ |
| Me | H | Me | Me | OCHF₂ |
| n-Pr | H | Me | Me | CF₃ |
| Me | H | Me | Me | C₂F₅ |
| Et | H | Me | Me | C₂F₅ |
| Me | H | Me | Et | CF₃ |
| Me | H | Me | n-Pr | CF₃ |
| Me | H | Me | i-Pr | CF₃ |
| Me | H | Me | Cl | CF₃ |
| Me | H | Me | F | CF₃ |
| Me | H | Me | Me | SMe |
| Me | H | Me | Me | OMe |
| Me | H | Me | Me | OEt |
| Me | H | Me | Me | n-C₃F₇ |
| Me | H | Me | Me | i-C₃F₇ |
| Me | H | Me | Me | Et |
| Me | H | Me | Me | OCF₂CHF₂ |
| Me | H | Me | Me | SCF₂CHF₂ |
| Me | H | Me | Me | SO₂ |
| Me | H | Me | Me | SO₂CF₃ |
| Me | H | Me | CF₃ | CF₃ |
| Me | H | Me | CF₃ | Me |
| Me | H | Me | OMe | CF₃ |
| Me | H | Me | H | CF₃ |
| Me | H | Me | H | OCHF₂ |
| Me | H | Me | H | C₂F₅ |
| Et | H | Me | H | C₂F₅ |
| Me | H | Me | H | OCF₃ |
| Me | H | Me | H | OCF₂CHF₂ |
| Me | H | Me | H | SCF₂CHF₂ |
| Me | H | Me | H | n-C₃F₇ |
| Me | H | Me | H | i-C₃F₇ |
| Me | H | Me | H | Br |
| Me | H | Me | H | Cl |
| Me | H | Me | H | SCF₃ |
| Me | H | Me | Ph | CF₃ |
| Me | H | Me | Ph | Cl |
| Me | H | Me | Ph | Br |
| Me | H | Me | 2-pyridyl | CF₃ |
| Me | H | Me | 2-pyridyl | Cl |
| Me | H | Me | 2-ClPh | CF₃ |
| Me | H | Me | 2-ClPh | OCF₃ |
| Me | H | Me | 2-ClPh | Br |
| Me | H | Me | 2-ClPh | Cl |
| Me | H | Me | 2-ClPh | SCHF₂ |
| Me | H | Me | 2-BrPh | CF₃ |
| Me | H | Me | 2-MePh | CF₃ |
| Me | H | Me | 2-CNPh | CF₃ |
| Me | H | Me | 2-FPh | CF₃ |
| Me | H | Me | 2,6-F₂Ph | CF₃ |
| Me | H | Me | 2,4-F₂Ph | CF₃ |
| Me | H | Me | 2,5-F₂Ph | CF₃ |
| Me | H | Me | 2-MeOPh | CF₃ |
| Me | H | Me | 3-Cl-2-pyridyl | CF₃ |
| Me | H | Me | 3-Cl-2-pyridyl | OCF₃ |
| Me | H | Me | 3-Cl-2-pyridyl | Br |
| Me | H | Me | 3-Cl-2-pyridyl | Cl |
| Me | H | Me | 3-Cl-2-pyridyl | SCHF₂ |
| Me | H | Me | 3-F-2-pyridyl | CF₃ |
| Me | H | Me | 3-CF₃-2- | CF₃ |
| | | | pyridyl | |
| Me | H | Me | 3-Me-2- | CF₃ |
| | | | pyridyl | |
| Me | H | Me | 3-Br-2-pyridyl | CF₃ |
| Me | H | Me | 3-Br-2-pyridyl | OCF₃ |
| Me | H | Me | 3-Br-2-pyridyl | Br |
| Me | H | Me | 3-Br-2-pyridyl | Cl |
| Me | Me | H | H | CF₃ |
| Me | Me | H | H | OCF₃ |
| Et | Me | H | H | OCF₃ |
| Me | Me | H | Me | Br |
| Me | Me | H | Et | Br |
| Me | Me | H | Me | Cl |
| Me | Me | H | Et | Cl |
| Me | Me | H | Me | I |
| Me | Me | H | Me | CF₃ |
| Me | Me | H | Me | OCF₃ |
| Et | Me | H | Me | CF₃ |
| Me | Me | H | Me | SCF₃ |
| Me | Me | H | Me | SCHF₂ |
| Me | Me | H | Me | OCHF₂ |
| n-Pr | Me | H | Me | CF₃ |
| Me | Me | H | Me | C₂F₅ |
| Et | Me | H | Me | C₂F₅ |
| Me | Me | H | Et | CF₃ |
| Me | Me | H | n-Pr | CF₃ |
| Me | Me | H | i-Pr | CF₃ |
| Me | Me | H | Cl | CF₃ |
| Me | Me | H | F | CF₃ |
| Me | Me | H | Me | SMe |
| Me | Me | H | Me | OMe |
| Me | Me | H | Me | OEt |
| Me | Me | H | Me | n-C₃F₇ |
| Me | Me | H | Me | i-C₃F₇ |
| Me | Me | H | Me | Et |
| Me | Me | H | Me | OCF₂CHF₂ |
| Me | Me | H | Me | SCF₂CHF₂ |
| Me | Me | H | Me | SO₂Me |
| Me | Me | H | Me | SO₂CF₃ |
| Me | Me | H | CF₃ | CF₃ |
| Me | Me | H | CF₃ | Me |
| Me | Me | H | OMe | CF₃ |
| Me | Me | H | H | CF₃ |
| Me | Me | H | H | OCHF₂ |
| Me | Me | H | H | C₂F₅ |
| Et | Me | H | H | C₂F₅ |
| Me | Me | H | H | OCF₃ |
| Me | Me | H | H | OCF₂CHF₂ |
| Me | Me | H | H | SCF₂CHF₂ |
| Me | Me | H | H | n-C₃F₇ |
| Me | Me | H | H | i-C₃F₇ |
| Me | Me | H | H | Br |
| Me | Me | H | H | Cl |
| Me | Me | H | H | SCF₃ |
| Me | Me | H | Ph | CF₃ |
| Me | Me | H | Ph | Cl |
| Me | Me | H | Ph | Br |
| Me | Me | H | 2-pyridyl | CF₃ |
| Me | Me | H | 2-pyridyl | Cl |
| Me | Me | H | 2-ClPh | CF₃ |
| Me | Me | H | 2-ClPh | OCF₃ |
| Me | Me | H | 2-ClPh | Br |
| Me | Me | H | 2-ClPh | Cl |
| Me | Me | H | 2-ClPh | SCHF₂ |
| Me | Me | H | 2-BrPh | CF₃ |
| Me | Me | H | 2-MePh | CF₃ |
| Me | Me | H | 2-CNPh | CF₃ |
| Me | Me | H | 2-FPh | CF₃ |
| Me | Me | H | 2,6-F₂Ph | CF₃ |
| Me | Me | H | 2,4-F₂Ph | CF₃ |
| Me | Me | H | 2,5-F₂Ph | CF₃ |
| Me | Me | H | 2-MeOPh | CF₃ |
| Me | Me | H | 3-Cl-2-pyridyl | CF₃ |
| Me | Me | H | 3-Cl-2-pyridyl | OCF₃ |
| Me | Me | H | 3-CF₃-2- | CF₃ |
| | | | pyridyl | |
| Me | Me | H | 3-Me-2- | CF₃ |
| | | | pyridyl | |
| Me | Me | H | 3-Cl-2-pyridyl | SCHF₂ |
| Me | Me | H | 3-F-2-pyridyl | CF₃ |
| Me | Me | H | 3-Cl-2-pyridyl | Br |
| Me | Me | H | 3-Cl-2-pyridyl | Cl |
| Me | Me | H | 3-Br-2-pyridyl | CF₃ |
| Me | Me | H | 3-Br-2-pyridyl | OCF₃ |
| Me | Me | H | 3-Br-2-pyridyl | Br |
| Me | Me | H | 3-Br-2-pyridyl | Cl |
| Me | Cl | H | Me | Br |
| Me | Cl | H | Et | Br |
| Me | Cl | H | Me | Cl |
| Me | Cl | H | Et | Cl |
| Me | Cl | H | Me | I |
| Me | Cl | H | Me | CF₃ |
| Me | Cl | H | Me | OCF₃ |
| Et | Cl | H | Me | CF₃ |
| Me | Cl | H | Me | SCF₃ |
| Me | Cl | H | Me | SCHF₂ |
| Me | Cl | H | Me | OCHF₂ |
| n-Pr | Cl | H | Me | CF₃ |
| Me | Cl | H | Me | C₂F₅ |
| Et | Cl | H | Me | C₂F₅ |
| Me | Cl | H | Et | CF₃ |
| Me | Cl | H | n-Pr | CF₃ |
| Me | Cl | H | i-Pr | CF₃ |
| Me | Cl | H | Cl | CF₃ |
| Me | Cl | H | F | CF₃ |
| Me | Cl | H | Me | SMe |
| Me | Cl | H | 3-Me-2- | CF₃ |
| | | | pyridyl | |
| Me | Cl | H | Me | OEt |
| Me | Cl | H | Me | n-C₃F₇ |
| Me | Cl | H | Me | i-C₃F₇ |
| Me | Cl | H | Me | Et |
| Me | Cl | H | Me | OCF₂CHF₂ |
| Me | Cl | H | Me | SCF₂CHF₂ |
| Me | Cl | H | Me | SO₂Me |
| Me | Cl | H | Me | SO₂CF₃ |
| Me | Cl | H | CF₃ | CF₃ |
| Me | Cl | H | CF₃ | Me |
| Me | Cl | H | OMe | CF₃ |
| Me | Cl | H | H | CF₃ |
| Me | Cl | H | H | OCHF₂ |
| Me | Cl | H | H | C₂F₅ |
| Et | Cl | H | H | C₂F₅ |
| Me | H | H | H | CF₃ |
| Me | H | H | H | OCF₃ |
| Et | H | H | H | OCF₃ |
| Me | H | H | Me | Br |
| Me | H | H | Et | Br |
| Me | H | H | Me | Cl |
| Me | H | H | Et | Cl |
| Me | H | H | Me | I |
| Me | H | H | Me | CF₃ |
| Me | H | H | Me | OCF₃ |
| Et | H | H | Me | CF₃ |
| Me | H | H | Me | SCF₃ |
| Me | H | H | Me | SCHF₂ |
| Me | H | H | Me | OCHF₂ |
| n-Pr | H | H | Me | CF₃ |
| Me | H | H | Me | C₂F₅ |
| Et | H | H | Me | C₂F₅ |
| Me | H | H | Et | CF₃ |
| Me | H | H | n-Pr | CF₃ |
| Me | H | H | i-Pr | CF₃ |
| Me | H | H | Cl | CF₃ |
| Me | H | H | F | CF₃ |
| Me | H | H | Me | SMe |
| Me | H | H | Me | OMe |
| Me | H | H | Me | OEt |
| Me | H | H | Me | n-C₃F₇ |
| Me | H | H | Me | i-C₃F₇ |
| Me | H | H | Me | Et |
| Me | H | H | Me | OCF₂CHF₂ |
| Me | H | H | Me | SCF₂CHF₂ |
| Me | Me | H | Me | SO₂Me |
| Me | H | H | Me | SO₂CF₃ |
| Me | H | H | CF₃ | CF₃ |
| Me | H | H | CF₃ | Me |
| Me | H | H | OMe | CF₃ |
| Me | H | H | H | CF₃ |
| Me | H | H | H | OCHF₂ |
| Me | H | H | H | C₂F₅ |
| Et | H | H | H | C₂F₅ |
| Me | H | H | H | OCF₃ |
| Me | H | H | H | OCF₂CHF₂ |
| Me | H | H | H | SCF₂CHF₂ |
| Me | H | H | H | n-C₃F₇ |
| Me | H | H | H | i-C₃F₇ |
| Me | H | H | H | Br |
| Me | H | H | H | Cl |
| Me | H | H | H | SCF₃ |
| Me | H | H | Ph | CF₃ |
| Me | H | H | Ph | Cl |
| Me | H | H | Ph | Br |
| Me | H | H | 2-pyridyl | CF₃ |
| Me | H | H | 2-pyridyl | Cl |
| Me | H | H | 2-ClPh | CF₃ |
| Me | H | H | 2-ClPh | OCF₃ |
| Me | H | H | 2-ClPh | Br |
| Me | H | H | 2-ClPh | Cl |
| Me | H | H | 2-ClPh | SCHF₂ |
| Me | H | H | 2-BrPh | CF₃ |
| Me | H | H | 2-MePh | CF₃ |
| Me | H | H | 2-CNPh | CF₃ |
| Me | H | H | 2-FPh | CF₃ |
| Me | H | H | 2,6-F₂Ph | CF₃ |
| Me | H | H | 2,4-F₂Ph | CF₃ |
| Me | H | H | 2,5-F₂Ph | CF₃ |
| Me | H | H | 2-MeOPh | CF₃ |
| Me | H | H | 3-Cl-2-pyridyl | CF₃ |
| Me | H | H | 3-Cl-2-pyridyl | OCF₃ |
| Me | H | H | 3-Cl-2-pyridyl | Br |
| Me | H | H | 3-Cl-2-pyridyl | Cl |
| Me | H | H | 3-Cl-2-pyridyl | SCHF₂ |
| Me | H | H | 3-F-2-pyridyl | CF₃ |
| Me | H | H | 3-CF₃-2- | CF₃ |
| | | | pyridyl | |
| Me | H | H | 3-Me-2- | CF₃ |
| | | | pyridyl | |
| Me | H | H | 3-Br-2-pyridyl | CF₃ |
| Me | H | H | 3-Br-2-pyridyl | OCF₃ |
| Me | H | H | 3-Br-2-pyridyl | Br |
| Me | H | H | 3-Br-2-pyridyl | Cl |
| Me | H | Cl | Et | Br |
| Me | H | Cl | Me | Cl |
| Me | H | Cl | Et | Cl |
| Me | H | Cl | Me | I |
| Me | H | Cl | Me | CF₃ |
| Me | H | Cl | Me | OCF₃ |
| Et | H | Cl | Me | CF₃ |
| Me | H | Cl | Me | SCF₃ |
| Me | H | Cl | Me | SCHF₂ |
| Me | H | Cl | Me | OCHF₂ |
| n-Pr | H | Cl | Me | CF₃ |
| Me | H | Cl | Me | C₂F₅ |
| Et | H | Cl | Me | C₂F₅ |
| Me | H | Cl | Et | CF₃ |
| Me | H | Cl | n-Pr | CF₃ |
| Me | H | Cl | i-Pr | CF₃ |
| Me | H | Cl | Cl | CF₃ |
| Me | H | Cl | F | CF₃ |
| Me | H | Cl | Me | SMe |
| Me | H | Cl | Me | OMe |
| Me | H | Cl | Me | OEt |
| Me | H | Cl | Me | n-C₃F₇ |
| Me | H | Cl | Me | i-C₃F₇ |
| Me | H | Cl | Me | Et |
| Me | H | Cl | Me | OCF₂CHF₂ |
| Me | H | Cl | Me | SCF₂CHF₂ |
| Me | H | Cl | Me | SO₂Me |
| Me | H | Cl | Me | SO₂CF₃ |
| Me | H | Cl | CF₃ | CF₃ |
| Me | H | Cl | CF₃ | Me |
| Me | H | Cl | OMe | CF₃ |
| Me | H | Cl | H | CF₃ |
| Me | H | Cl | H | OCHF₂ |
| Me | H | Cl | H | C₂F₅ |
| Et | H | Cl | H | C₂F₅ |
| Me | H | Cl | H | OCF₃ |
| Me | H | Cl | H | OCF₂CHF₂ |
| Me | H | Cl | H | SCF₂CHF₂ |
| Me | H | Cl | H | n-C₃F₇ |
| Me | H | Cl | H | i-C₃F₇ |
| Me | H | Cl | H | Br |
| Me | H | Cl | H | Cl |
| Me | H | Cl | H | SCF₃ |
| Me | H | Cl | Ph | CF₃ |
| Me | H | Cl | Ph | Cl |
| Me | H | Cl | Ph | Br |
| Me | H | Cl | 2-pyridyl | CF₃ |
| Me | H | Cl | 2-pyridyl | Cl |
| Me | H | Cl | 2-ClPh | CF₃ |
| Me | H | Cl | 2-ClPh | OCF₃ |
| Me | H | Cl | 2-ClPh | Br |
| Me | H | Cl | 2-ClPh | Cl |
| Me | H | Cl | 2-ClPh | SCHF₂ |
| Me | H | Cl | 2-BrPh | CF₃ |
| Me | H | Cl | 2-MePh | CF₃ |
| Me | H | Cl | 2-CNPh | CF₃ |
| Me | H | Cl | 2-FPh | CF₃ |
| Me | H | Cl | 2,6-F₂Ph | CF₃ |
| Me | H | Cl | 2,4-F₂Ph | CF₃ |
| Me | H | Cl | 2,5-F₂Ph | CF₃ |
| Me | H | Cl | 2-MeOPh | CF₃ |
| Me | H | Cl | 3-Cl-2-pyridyl | CF₃ |
| Me | H | Cl | 3-Cl-2-pyridyl | OCF₃ |
| Me | H | Cl | 3-Cl-2-pyridyl | Br |
| Me | H | Cl | 3-Cl-2-pyridyl | Cl |
| Me | H | Cl | 3-Cl-2-pyridyl | SCHF₂ |
| Me | H | Cl | 3-F-2-pyridyl | CF₃ |
| Me | H | Cl | 3-CF₃-2- | CF₃ |
| | | | pyridyl | |
| Me | H | Cl | 3-Me-2- | CF₃ |
| | | | pyridyl | |
| Me | H | Cl | 3-Br-2-pyridyl | CF₃ |
| Me | H | Cl | 3-Br-2-pyridyl | OCF₃ |
| Me | H | Cl | 3-Br-2-pyridyl | Br |
| Me | H | Cl | 3-Br-2-pyridyl | Cl |
| Me | Cl | H | Ph | CF₃ |
| Me | Cl | H | Ph | Cl |
| Me | Cl | H | Ph | Br |
| Me | Cl | H | 2-pyridyl | CF₃ |
| Me | Cl | H | 2-pyridyl | Cl |
| Me | Cl | H | 2-ClPh | CF₃ |
| Me | Cl | H | 2-ClPh | OCF₃ |
| Me | Cl | H | 2-ClPh | Br |
| Me | Cl | H | 2-ClPh | Cl |
| Me | Cl | H | 2-ClPh | SCHF₂ |
| Me | Cl | H | 2-BrPh | CF₃ |
| Me | Cl | H | 2-MePh | CF₃ |
| Me | Cl | H | 2-CNPh | CF₃ |
| Me | Cl | H | 2-FPh | CF₃ |
| Me | Cl | H | 2,6-F₂Ph | CF₃ |
| Me | Cl | H | 2,4-F₂Ph | CF₃ |
| Me | Cl | H | 2,5-F,Ph | CF₃ |
| Me | Cl | H | 2-MeOPh | CF₃ |
| Me | Cl | H | 3-Cl-2-pyridyl | CF₃ |
| Me | Cl | H | 3-Cl-2-pyridyl | OCF₃ |
| Me | Cl | H | 3-Cl-2-pyridyl | Br |
| Me | Cl | H | 3-Cl-2-pyridyl | Cl |
| Me | Cl | H | 3-Cl-2-pyridyl | SCHF₂ |
| Me | Cl | H | 3-F-2-pyridyl | CF₃ |
| Me | Cl | H | 3-CF₃-2- | CF₃ |
| | | | pyridyl | |
| Me | Cl | H | 3-Br-2-pyridyl | CF₃ |
| Me | Cl | H | 3-Br-2-pyridyl | OCF₃ |
| Me | Cl | H | 3-Br-2-pyridyl | Br |
| Me | Cl | H | 3-Br-2-pyridyl | Cl |
| Me | Cl | H | H | OCF₃ |
| Me | Cl | H | H | OCF₂CHF₂ |
| Me | Cl | H | H | SCF₂CHF₂ |
| Me | Cl | H | H | n-C₃F₇ |
| Me | Cl | H | H | i-C₃F₇ |
| Me | Cl | H | H | Br |
| Me | Cl | H | H | Cl |
| Me | Cl | H | H | SCF₃ |
| Me | Cl | H | Ph | CF₃ |
| Me | Cl | H | Me | OMe |

TABLE 2

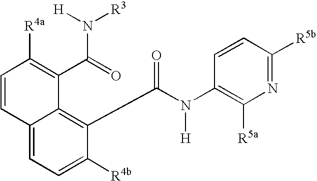

| R³ | R^{4a} | R^{4b} | R^{5a} | R^{5b} |
|---|---|---|---|---|
| Me | H | Me | H | CF₃ |
| Me | H | Me | H | OCF₃ |
| Et | H | Me | H | OCF₃ |
| Me | H | Me | Me | Br |
| Me | H | Me | Et | Br |
| Me | H | Me | Me | Cl |
| Me | H | Me | Et | Cl |
| Me | H | Me | Me | I |
| Me | H | Me | Me | CF₃ |
| Me | H | Me | Me | OCF₃ |
| Et | H | Me | Me | CF₃ |
| Me | H | Me | Me | SCF₃ |
| Me | H | Me | Me | SCHF₂ |
| Me | H | Me | Me | OCHF₂ |
| n-Pr | H | Me | Me | CF₃ |
| Me | H | Me | Me | C₂F₅ |
| Et | H | Me | Me | C₂F₅ |
| Me | H | Me | Et | CF₃ |
| Me | H | Me | n-Pr | CF₃ |
| Me | H | Me | i-Pr | CF₃ |
| Me | H | Me | Cl | CF₃ |
| Me | H | Me | F | CF₃ |
| Me | H | Me | Me | SMe |
| Me | H | Me | Me | OMe |
| Me | H | Me | Me | OEt |
| Me | H | Me | Me | n-C₃F₇ |
| Me | H | Me | Me | i-C₃F₇ |
| Me | H | Me | Me | Et |
| Me | H | Me | Me | OCF₂CHF₂ |
| Me | H | Me | Me | SCF₂CHF₂ |
| Me | H | Me | Me | SO₂Me |
| Me | H | Me | Me | SO₂CF₃ |
| Me | H | Me | CF₃ | CF₃ |
| Me | H | Me | CF₃ | Me |
| Me | H | Me | OMe | CF₃ |
| Me | H | Me | H | CF₃ |
| Me | H | Me | H | OCHF₂ |
| Me | H | Me | H | C₂F₅ |
| Et | H | Me | H | C₂F₅ |
| Me | H | Me | H | OCF₃ |
| Me | H | Me | H | OCF₂CHF₂ |
| Me | H | Me | H | SCF₂CHF₂ |
| Me | H | Me | H | n-C₃F₇ |
| Me | H | Me | H | i-C₃F₇ |
| Me | H | Me | H | Br |
| Me | H | Me | H | Cl |
| Me | H | Me | H | SCF₃ |
| Me | H | Me | Ph | CF₃ |
| Me | H | Me | Ph | Cl |
| Me | H | Me | Ph | Br |
| Me | H | Me | 2-pyridyl | CF₃ |
| Me | H | Me | 2-pyridyl | Cl |
| Me | H | Me | 2-ClPh | CF₃ |
| Me | H | Me | 2-ClPh | OCF₃ |
| Me | H | Me | 2-ClPh | Br |
| Me | H | Me | 2-ClPh | Cl |
| Me | H | Me | 2-ClPh | SCHF₂ |
| Me | H | Me | 2-BrPh | CF₃ |
| Me | H | Me | 2-MePh | CF₃ |
| Me | H | Me | 2-CNPh | CF₃ |
| Me | H | Me | 2-FPh | CF₃ |
| Me | H | Me | 2,6-F₂Ph | CF₃ |
| Me | H | Me | 2,4-F₂Ph | CF₃ |
| Me | H | Me | 2,5-F₂Ph | CF₃ |
| Me | H | Me | 2-MeOPh | CF₃ |
| Me | H | Me | 3-Cl-2-pyridyl | CF₃ |
| Me | H | Me | 3-Cl-2-pyridyl | OCF₃ |
| Me | H | Me | 3-Cl-2-pyridyl | Br |
| Me | H | Me | 3-Cl-2-pyridyl | Cl |
| Me | H | Me | 3-Cl-2-pyridyl | SCHF₂ |
| Me | H | Me | 3-F-2-pyridyl | CF₃ |
| Me | H | Me | 3-CF₃₋₂₋ | CF₃ |
| | | | pyridyl | |
| Me | H | Me | 3-Me-2- | CF₃ |
| | | | pyridyl | |
| Me | H | Me | 3-Br-2-pyridyl | CF₃ |
| Me | H | Me | 3-Br-2-pyridyl | OCF₃ |
| Me | H | Me | 3-Br-2-pyridyl | Br |
| Me | H | Me | 3-Br-2-pyridyl | Cl |
| Me | Me | H | H | CF₃ |
| Me | Me | H | H | OCF₃ |
| Et | Me | H | H | OCF₃ |
| Me | Me | H | Me | Br |
| Me | Me | H | Et | Br |
| Me | Me | H | Me | Cl |
| Me | Me | H | Cl | Et |
| Me | Me | H | Me | I |
| Me | Me | H | Me | CF₃ |
| Me | Me | H | Me | OCF₃ |
| Et | Me | H | Me | CF₃ |
| Me | Me | H | Me | SCF₃ |
| Me | Me | H | Me | SCHF₂ |
| Me | Me | H | Me | OCHF₂ |
| n-Pr | Me | H | Me | CF₃ |
| Me | Me | H | Me | C₂F₅ |
| Et | Me | H | Me | C₂F₅ |
| Me | Me | H | Et | CF₃ |
| Me | Me | H | n-Pr | CF₃ |
| Me | Me | H | i-Pr | CF₃ |
| Me | Me | H | Cl | CF₃ |
| Me | Me | H | F | CF₃ |
| Me | Me | H | Me | SMe |
| Me | Me | H | Me | OMe |
| Me | Me | H | Me | OEt |
| Me | Me | H | Me | n-C₃F₇ |
| Me | Me | H | Me | i-C₃F₇ |
| Me | Me | H | Me | Et |
| Me | Me | H | Me | OCF₂CHF₂ |
| Me | Me | H | Me | SCF₂CHF₂ |
| Me | Me | H | Me | SO₂Me |
| Me | Me | H | Me | SO₂CF₃ |
| Me | Me | H | CF₃ | CF₃ |
| Me | Me | H | CF₃ | Me |
| Me | Me | H | OMe | CF₃ |
| Me | Me | H | H | CF₃ |
| Me | Me | H | H | OCHF₂ |
| Me | Me | H | H | C₂F₅ |
| Et | Me | H | H | C₂F₅ |
| Me | Me | H | H | OCF₃ |
| Me | Me | H | H | OCF₂CHF₂ |
| Me | Me | H | H | SCF₂CHF₂ |
| Me | Me | H | H | n-C₃F₇ |
| Me | Me | H | H | i-C₃F₇ |
| Me | Me | H | H | Br |
| Me | Me | H | H | Cl |
| Me | Me | H | H | SCF₃ |
| Me | Me | H | Ph | CF₃ |
| Me | Me | H | Ph | Cl |
| Me | Me | H | Ph | Br |
| Me | Me | H | 2-pyridyl | CF₃ |
| Me | Me | H | 2-pyridyl | Cl |
| Me | Me | H | 2-ClPh | CF₃ |
| Me | Me | H | 2-ClPh | OCF₃ |
| Me | Me | H | 2-ClPh | Br |
| Me | Me | H | 2-ClPh | Cl |
| Me | Me | H | 2-ClPh | SCHF₂ |
| Me | Me | H | 2-BrPh | CF₃ |
| Me | Me | H | 2-MePh | CF₃ |
| Me | Me | H | 2-CNPh | CF₃ |
| Me | Me | H | 2-FPh | CF₃ |
| Me | Me | H | 2,5-F₂Ph | CF₃ |
| Me | Me | H | 2,4-F₂Ph | CF₃ |
| Me | Me | H | 2,5-F₂Ph | CF₃ |
| Me | Me | H | 2-MeOPh | CF₃ |
| Me | Me | H | 3-Cl-2-pyridyl | CF₃ |
| Me | Me | H | 3-Cl-2-pyridyl | OCF₃ |
| Me | Me | H | 3-CF₃₋₂₋ | CF₃ |
| | | | pyridyl | |
| Me | Me | H | 3-Me-2- | CF₃ |
| | | | pyridyl | |
| Me | Me | H | 3-Cl-2-pyridyl | SCHF₂ |
| Me | Me | H | 3-F-2-pyridyl | CF₃ |
| Me | Me | H | 3-Cl-2-pyridyl | Br |
| Me | Me | H | 3-Cl-2-pyridyl | Cl |
| Me | Me | H | 3-Br-2-pyridyl | CF₃ |
| Me | Me | H | 3-Br-2-pyridyl | OCF₃ |
| Me | Me | H | 3-Br-2-pyridyl | Br |
| Me | Me | H | 3-Br-2-pyridyl | Cl |
| Me | Cl | H | Me | Br |
| Me | Cl | H | Et | Br |
| Me | Cl | H | Me | Cl |
| Me | Cl | H | Et | Cl |
| Me | Cl | H | Me | Cl |
| Me | Cl | H | Me | CF₃ |
| Me | Cl | H | Me | OCF₃ |
| Et | Cl | H | Me | CF₃ |
| Me | Cl | H | Me | SCF₃ |
| Me | Cl | H | Me | SCHF₂ |
| Me | Cl | H | Me | OCHF₂ |
| n-Pr | Cl | H | Me | CF₃ |
| Me | Cl | H | Me | C₂F₅ |
| Et | Cl | H | Me | C₂F₅ |
| Me | Cl | H | Et | CF₃ |
| Me | Cl | H | n-Pr | CF₃ |
| Me | Cl | H | i-Pr | CF₃ |
| Me | Cl | H | Cl | CF₃ |
| Me | Cl | H | F | CF₃ |
| Me | Cl | H | Me | SMe |
| Me | Cl | H | Me | OMe |
| Me | Cl | H | Me | OEt |
| Me | Cl | H | Me | n-C₃F₇ |
| Me | Cl | H | Me | i-C₃F₇ |
| Me | Cl | H | Me | Et |
| Me | Cl | H | Me | OCF₂CHF₂ |
| Me | Cl | H | Me | SCF₂CHF₂ |
| Me | Cl | H | Me | SO₂Me |
| Me | Cl | H | Me | SO₂CF₃ |
| Me | Cl | H | 3-CF₃₋₂₋ | CF₃ |
| | | | pyridyl | |
| Me | Cl | H | CF₃ | Me |
| Me | Cl | H | OMe | CF₃ |
| Me | Cl | H | H | CF₃ |
| Me | Cl | H | H | OCHF₂ |
| Me | Cl | H | H | C₂F₅ |
| Me | H | H | H | CF₃ |
| Me | H | H | H | OCF₃ |
| Et | H | H | H | OCF₃ |
| Me | H | H | Me | Br |
| Me | H | H | Et | Br |
| Me | H | H | Me | Cl |
| Me | H | H | Et | Cl |
| Me | H | H | Me | I |
| Me | H | H | Me | CF₃ |
| Me | H | H | Me | OCF₃ |
| Et | H | H | Me | CF₃ |
| Me | H | H | Me | SCF₃ |
| Me | H | H | Me | SCHF₂ |
| Me | H | H | Me | OCHF₂ |
| n-Pr | H | H | Me | CF₃ |
| Me | H | H | Me | C₂F₅ |
| Et | H | H | Me | C₂F₅ |
| Me | H | H | Et | CF₃ |
| Me | H | H | n-Pr | CF₃ |
| Me | H | H | i-Pr | CF₃ |
| Me | H | H | Cl | CF₃ |
| Me | H | H | F | CF₃ |
| Me | H | H | Me | SMe |
| Me | H | H | Me | OMe |
| Me | H | H | Me | OEt |
| Me | H | H | Me | n-C₃F₇ |
| Me | H | H | Me | i-C₃F₇ |
| Me | H | H | Me | Et |
| Me | H | H | Me | OCF₂CHF₂ |
| Me | H | H | Me | SCF₂CHF₂ |
| Me | H | H | Me | SO₂Me |
| Me | H | H | Me | SO₂CF₃ |
| Me | H | H | CF₃ | CF₃ |
| Me | H | H | CF₃ | Me |
| Me | H | H | OMe | CF₃ |
| Me | H | H | H | CF₃ |
| Me | H | H | H | OCHF₂ |
| Me | H | H | H | C₂F₅ |
| Et | H | H | H | C₂F₅ |
| Me | H | H | H | OCF₃ |
| Me | H | H | H | OCF₂CHF₂ |
| Me | H | H | H | SCF₂CHF₂ |
| Me | H | H | H | n-C₃F₇ |
| Me | H | H | H | i-C₃F₇ |
| Me | H | H | H | Br |
| Me | H | H | H | Cl |
| Me | H | H | H | SCF₃ |
| Me | H | H | Ph | CF₃ |
| Me | H | H | Ph | Cl |
| Me | H | H | Ph | Br |
| Me | H | H | 2-pyridyl | CF₃ |
| Me | H | H | 2-pyridyl | Cl |
| Me | H | H | 2-ClPh | CF₃ |
| Me | H | H | 2-ClPh | OCF₃ |
| Me | H | H | 2-ClPh | Br |
| Me | H | H | 2-ClPh | Cl |
| Me | H | H | 2-ClPh | SCHF₂ |
| Me | H | H | 2-BrPh | CF₃ |
| Me | H | H | 2-MePh | CF₃ |
| Me | H | H | 2-CNPh | CF₃ |
| Me | H | H | 2-FPh | CF₃ |
| Me | H | H | 2,6-F₂Ph | CF₃ |
| Me | H | H | 2,4-F₂Ph | CF₃ |
| Me | H | H | 2,5-F₂Ph | CF₃ |
| Me | H | H | 2-MeOPh | CF₃ |
| Me | H | H | 3-Cl-2-pyridyl | CF₃ |
| Me | H | H | 3-Cl-2-pyridyl | OCF₃ |
| Me | H | H | 3-Cl-2-pyridyl | Br |
| Me | H | H | 3-Cl-2-pyridyl | Cl |
| Me | H | H | 3-Cl-2-pyridyl | SCHF₂ |
| Me | H | H | 3-F-2-pyridyl | CF₃ |
| Me | H | H | 3-CF₃-2- | CF₃ |
| | | | pyridyl | |
| Me | H | H | 3-Me-2- | CF₃ |
| | | | pyridyl | |
| Me | H | H | 3-Br-2-pyridyl | CF₃ |
| Me | H | H | 3-Br-2-pyridyl | OCF₃ |
| Me | H | H | 3-Br-2-pyridyl | Br |
| Me | H | H | 3-Br-2-pyridyl | Cl |
| Me | H | Cl | Et | Br |
| Me | H | Cl | Me | Cl |
| Me | H | Cl | Et | Cl |
| Me | H | Cl | Me | I |
| Me | H | Cl | Me | CF₃ |
| Me | H | Cl | Me | OCF₃ |
| H | Cl | Me | CF₃ | |
| Me | H | Cl | Me | SCF₃ |
| Me | H | Cl | Me | SCHF₂ |
| Me | H | Cl | Me | OCHF₂ |
| n-Pr | H | Cl | Me | CF₃ |
| Me | H | Cl | Me | C₂F₅ |
| Et | H | Cl | Me | C₂F₅ |
| Me | H | Cl | Et | CF₃ |
| Me | H | Cl | n-Pr | CF₃ |
| Me | H | Cl | i-Pr | CF₃ |
| Me | H | Cl | Cl | CF₃ |
| Me | H | Cl | F | CF₃ |
| Me | H | Cl | Me | SMe |
| Me | H | Cl | Me | OMe |
| Me | H | Cl | Me | OEt |
| Me | H | Cl | Me | n-C₃F₇ |
| Me | H | Cl | Me | i-C₃F₇ |
| Me | H | Cl | Me | Et |
| Me | H | Cl | Me | OCF₂CHF₂ |
| Me | H | Cl | Me | SCF₂CHF₂ |
| Me | H | Cl | Me | SO₂Me |
| Me | H | Cl | Me | SO₂CF₃ |
| Me | H | Cl | CF₃ | CF₃ |
| Me | H | Cl | CF₃ | Me |
| Me | H | Cl | OMe | CF₃ |
| Me | H | Cl | H | CF₃ |
| Me | H | Cl | H | OCHF₂ |
| Me | H | Cl | H | C₂F₅ |
| Et | H | Cl | H | C₂F₅ |
| Me | H | Cl | H | OCF₃ |
| Me | H | Cl | H | OCF₂CHF₂ |
| Me | H | Cl | H | SCF₂CHF₂ |
| Me | H | Cl | H | n-C₃F₇ |
| Me | H | Cl | H | i-C₃F₇ |
| Me | H | Cl | H | Br |
| Me | H | Cl | H | Cl |
| Me | H | Cl | H | SCF₃ |
| Me | H | Cl | Ph | CF₃ |
| Me | H | Cl | Ph | Cl |
| Me | H | Cl | Ph | Br |
| Me | H | Cl | 2-pyridyl | CF₃ |
| Me | H | Cl | 2-pyridyl | Cl |
| Me | H | Cl | 2-ClPh | CF₃ |
| Me | H | Cl | 2-ClPh | OCF₃ |
| Me | H | Cl | 2-ClPh | Br |
| Me | H | Cl | 2-ClPh | Cl |
| Me | H | Cl | 2-ClPh | SCHF₂ |
| Me | H | Cl | 2-BrPh | CF₃ |
| Me | H | Cl | 2-MePh | CF₃ |
| Me | H | Cl | 2-CNPh | CF₃ |
| Me | H | Cl | 2-FPh | CF₃ |
| Me | H | Cl | 2,6-FPh | CF₃ |
| Me | H | Cl | 2,4-F₂Ph | CF₃ |
| Me | H | Cl | 2,5-F₂Ph | CF₃ |
| Me | H | Cl | 2-MeOPh | CF₃ |
| Me | H | Cl | 3-Cl-2-pyridyl | CF₃ |
| Me | H | Cl | 3-Cl-2-pyridyl | OCF₃ |
| Me | H | Cl | 3-Cl-2-pyridyl | Br |
| Me | H | Cl | 3-Cl-2-pyridyl | Cl |
| Me | H | Cl | 3-Cl-2-pyridyl | SCHF₂ |
| Me | H | Cl | 3-F-2-pyridyl | CF₃ |
| Me | H | Cl | 3-CF₃₋₂₋ | CF₃ |
| | | | pyridyl | |
| Me | H | Cl | 3-Me-2- | CF₃ |
| | | | pyridyl | |
| Me | H | Cl | 3-Br-2-pyridyl | CF₃ |
| Me | H | Cl | 3-Br-2-pyridyl | OCF₃ |
| Me | H | Cl | 3-Br-2-pyridyl | Br |
| Me | H | Cl | 3-Br-2-pyridyl | Cl |
| Me | Cl | H | H | OCF₃ |
| Me | Cl | H | H | OCF₂CHF₂ |
| Me | Cl | H | H | SCF₂CHF₂ |
| Me | Cl | H | H | n-C₃F₇ |
| Me | Cl | H | H | i-C₃F₇ |
| Me | Cl | H | H | Br |
| Me | Cl | H | H | Cl |
| Me | Cl | H | H | SCF₃ |
| Me | Cl | H | Ph | CF₃ |
| Me | Cl | H | Ph | Cl |
| Me | Cl | H | Ph | Br |
| Me | Cl | H | 2-pyridyl | CF₃ |
| Me | Cl | H | 2-pyridyl | Cl |
| Me | Cl | H | 2-ClPh | CF₃ |
| Me | Cl | H | 2-ClPh | OCF₃ |
| Me | Cl | H | 2-ClPh | Br |
| Me | Cl | H | 2-ClPh | Cl |
| Me | Cl | H | 2-ClPh | SCHF₂ |
| Me | Cl | H | 2-BrPh | CF₃ |
| Me | Cl | H | 2-MePh | CF₃ |
| Me | Cl | H | 2-CNPh | CF₃ |
| Me | Cl | H | 2-FPh | CF₃ |
| Me | Cl | H | 2,5-F₂Ph | CF₃ |
| Me | Cl | H | 2,4-F₂Ph | CF₃ |
| Me | Cl | H | 2,5-F₂Ph | CF₃ |
| Me | Cl | H | 2-MeOPh | CF₃ |
| Me | Cl | H | 3-Cl-2-pyridyl | CF₃ |
| Me | Cl | H | 3-Cl-2-pyridyl | OCF₃ |
| Me | Cl | H | 3-Cl-2-pyridyl | Br |
| Me | Cl | H | 3-Cl-2-pyridyl | Cl |
| Me | Cl | H | 3-Cl-2-pyridyl | SCHF₂ |
| Me | Cl | H | 3-F-2-pyridyl | CF₃ |
| Me | Cl | H | CF₃ | CF₃ |
| Me | Cl | H | 3-Me-2- | CF₃ |
| | | | pyridyl | |
| Me | Cl | H | 3-Br-2-pyridyl | CF₃ |
| Me | Cl | H | 3-Br-2-pyridyl | OCF₃ |
| Me | Cl | H | 3-Br-2-pyridyl | Br |
| Me | Cl | H | 3-Br-2-pyridyl | Cl |
| Et | Cl | H | H | C₂F₅ |

TABLE 3

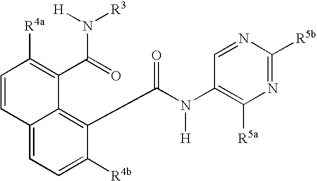

| R³ | R^{4a} | R^{4b} | R^{5a} | R^{5b} |
|---|---|---|---|---|
| Me | H | Me | H | CF₃ |
| Me | H | Me | H | OCF₃ |
| Et | H | Me | H | OCF₃ |
| Me | H | Me | Me | Br |
| Me | H | Me | Et | Br |
| Me | H | Me | Me | Cl |
| Me | H | Me | Et | Cl |
| Me | H | Me | Me | I |
| Me | H | Me | Me | CF₃ |
| Me | H | Me | Me | OCF₃ |
| Et | H | Me | Me | CF₃ |
| Me | H | Me | Me | SCF₃ |
| Me | H | Me | Me | SCHF₂ |
| Me | H | Me | Me | OCHF₂ |
| n-Pr | H | Me | Me | CF₃ |
| Me | H | Me | Me | C₂F₅ |
| Et | H | Me | Me | C₂F₅ |
| Me | H | Me | Et | CF₃ |
| Me | H | Me | n-Pr | CF₃ |
| Me | H | Me | i-Pr | CF₃ |
| Me | H | Me | Cl | CF₃ |
| Me | H | Me | F | CF₃ |
| Me | H | Me | Me | SMe |
| Me | H | Me | Me | OMe |
| Me | H | Me | Me | OEt |
| Me | H | Me | Me | n-C₃F₇ |
| Me | H | Me | Me | i-C₃F₇ |
| Me | H | Me | Me | Et |
| Me | H | Me | Me | OCF₂CHF₂ |
| Me | H | Me | Me | SCF₂CHF₂ |
| Me | H | Me | Me | SO₂Me |
| Me | H | Me | Me | SO₂CF₃ |
| Me | H | Me | CF₃ | CF₃ |
| Me | H | Me | CF₃ | Me |
| Me | H | Me | OMe | CF₃ |
| Me | H | Me | H | CF₃ |
| Me | H | Me | H | OCHF₂ |
| Me | H | Me | H | C₂F₅ |
| Et | H | Me | H | C₂F₅ |
| Me | H | Me | H | OCF₃ |
| Me | H | Me | H | OCF₂CHF₂ |
| Me | H | Me | H | SCF₂CHF₂ |
| Me | H | Me | H | n-C₃F₇ |
| Me | H | Me | H | i-C₃F₇ |
| Me | H | Me | H | Br |
| Me | H | Me | H | Cl |
| Me | H | Me | H | SCF₃ |
| Me | H | Me | Ph | CF₃ |
| Me | H | Me | Ph | Cl |
| Me | H | Me | Ph | Br |
| Me | H | Me | 2-pyridyl | CF₃ |
| Me | H | Me | 2-pyridyl | Cl |
| Me | H | Me | 2-ClPh | CF₃ |
| Me | H | Me | 2-ClPh | OCF₃ |
| Me | H | Me | 2-ClPh | Br |
| Me | H | Me | 2-ClPh | Cl |
| Me | H | Me | 2-ClPh | SCHF₂ |
| Me | H | Me | 2-BrPh | CF₃ |
| Me | H | Me | 2-MePh | CF₃ |
| Me | H | Me | 2-CNPh | CF₃ |
| Me | H | Me | 2-FPh | CF₃ |
| Me | H | Me | 2,6-F₂Ph | CF₃ |
| Me | H | Me | 2,4-F₂Ph | CF₃ |
| Me | H | Me | 2,5-F₂Ph | CF₃ |
| Me | H | Me | 2-MeOPh | CF₃ |
| Me | H | Me | 3-Cl-2-pyridyl | CF₃ |
| Me | H | Me | 3-Cl-2-pyridyl | OCF₃ |
| Me | H | Me | 3-Cl-2-pyridyl | Br |
| Me | H | Me | 3-Cl-2-pyridyl | Cl |
| Me | H | Me | 3-Cl-2-pyridyl | SCHF₂ |
| Me | H | Me | 3-F-2-pyridyl | CF₃ |
| Me | H | Me | 3-CF₃-2- | CF₃ |
| | | | pyridyl | |
| Me | H | Me | 3-Me-2- | CF₃ |
| | | | pyridyl | |
| Me | H | Me | 3-Br-2-pyridyl | CF₃ |
| Me | H | Me | 3-Br-2-pyridyl | OCF₃ |
| Me | H | Me | 3-Br-2-pyridyl | Br |
| Me | H | Me | 3-Br-2-pyridyl | Cl |
| Me | Me | H | H | CF₃ |
| Me | Me | H | H | OCF₃ |
| Et | Me | H | H | OCF₃ |
| Me | Me | H | Me | Br |
| Me | Me | H | Et | Br |
| Me | Me | H | Me | Cl |
| Me | Me | H | Et | Cl |
| Me | Me | H | Me | I |
| Me | Me | H | Me | CF₃ |
| Me | Me | H | Me | OCF₃ |
| Et | Me | H | Me | CF₃ |
| Me | Me | H | Me | SCF₃ |
| Me | Me | H | Me | SCHF₂ |
| Me | Me | H | Me | OCHF₂ |
| n-Pr | Me | H | Me | CF₃ |
| Me | Me | H | Me | C₂F₅ |
| Et | Me | H | Me | C₂F₅ |
| Me | Me | H | Et | CF₃ |
| Me | Me | H | n-Pr | CF₃ |
| Me | Me | H | i-Pr | CF₃ |
| Me | Me | H | Cl | CF₃ |
| Me | Me | H | F | CF₃ |
| Me | Me | H | Me | SMe |
| Me | Me | H | Me | OMe |
| Me | Me | H | Me | OEt |
| Me | Me | H | Me | n-C₃F₇ |
| Me | Me | H | Me | i-C₃F₇ |
| Me | Me | H | Me | Et |
| Me | Me | H | Me | OCF₂CHF₂ |
| Me | Me | H | Me | SCF₂CHF₂ |
| Me | Me | H | Me | SO₂Me |
| Me | Me | H | Me | SO₂CF₃ |
| Me | Me | H | CF₃ | CF₃ |
| Me | Me | H | CF₃ | Me |
| Me | Me | H | OMe | CF₃ |
| Me | Me | H | H | CF₃ |
| Me | Me | H | H | OCHF₂ |
| Me | Me | H | H | C₂F₅ |
| Et | Me | H | H | C₂F₅ |
| Me | Me | H | H | OCF₃ |
| Me | Me | H | H | OCF₂CHF₂ |
| Me | Me | H | H | SCF₂CHF₂ |
| Me | Me | H | H | n-C₃F₇ |
| Me | Me | H | H | i-C₃F₇ |
| Me | Me | H | H | Br |
| Me | Me | H | H | Cl |
| Me | Me | H | H | SCF₃ |
| Me | Me | H | Ph | CF₃ |
| Me | Me | H | Ph | Cl |
| Me | Me | H | Ph | Br |
| Me | Me | H | 2-pyridyl | CF₃ |
| Me | Me | H | 2-pyridyl | Cl |
| Me | Me | H | 2-ClPh | CF₃ |
| Me | Me | H | 2-ClPh | OCF₃ |
| Me | Me | H | 2-ClPh | Br |
| Me | Me | H | 2-ClPh | Cl |
| Me | Me | H | 2-ClPh | SCHF₂ |
| Me | Me | H | 2-BrPh | CF₃ |
| Me | Me | H | 2-MePh | CF₃ |
| Me | Me | H | 2-CNPh | CF₃ |
| Me | Me | H | 2-FPh | CF₃ |
| Me | Me | H | 2,6-F₂Ph | CF₃ |
| Me | Me | H | 2,4-F₂Ph | CF₃ |
| Me | Me | H | 2,5-F₂Ph | CF₃ |
| Me | Me | H | 2-MeOPh | CF₃ |
| Me | Me | H | 3-Cl-2-pyridyl | CF₃ |
| Me | Me | H | 3-Cl-2-pyridyl | OCF₃ |
| Me | Me | H | 3-CF₃-2- | CF₃ |
| | | | pyridyl | |
| Me | Me | H | 3-Me-2- | CF₃ |
| | | | pyridyl | |
| Me | Me | H | 3-Cl-2-pyridyl | SCHF₂ |
| Me | Me | H | 3-F-2-pyridyl | CF₃ |
| Me | Me | H | 3-Cl-2-pyridyl | Br |
| Me | Me | H | 3-Cl-2-pyridyl | Cl |
| Me | Me | H | 3-Br-2-pyridyl | CF₃ |
| Me | Me | H | 3-Br-2-pyridyl | OCF₃ |
| Me | Me | H | 3-Br-2-pyridyl | Br |
| Me | Me | H | 3-Br-2-pyridyl | Cl |
| Me | Cl | H | Me | Br |
| Me | Cl | H | Et | Br |
| Me | Cl | H | Me | Cl |
| Me | Cl | H | Et | Cl |
| Me | Cl | H | Me | I |
| Me | Cl | H | Me | CF₃ |
| Me | Cl | H | Me | OCF₃ |
| Et | Cl | H | Me | CF₃ |
| Me | Cl | H | Me | SCF₃ |
| Me | Cl | H | Me | SCHF₂ |
| Me | Cl | H | Me | OCHF₂ |
| n-Pr | Cl | H | Me | CF₃ |
| Me | Cl | H | Me | C₂F₅ |
| Et | Cl | H | Me | C₂F₅ |
| Me | Cl | H | Et | CF₃ |
| Me | Cl | H | n-Pr | CF₃ |
| Me | Cl | H | i-Pr | CF₃ |
| Me | Cl | H | Cl | CF₃ |
| Me | Cl | H | F | CF₃ |
| Me | Cl | H | Me | SMe |
| Me | Cl | H | Me | OMe |
| Me | Cl | H | Me | OEt |
| Me | Cl | H | Me | n-C₃F₇ |
| Me | Cl | H | Me | i-C₃F₇ |
| Me | Cl | H | Me | Et |
| Me | Cl | H | Me | OCF₂CHF₂ |
| Me | Cl | H | Me | SCF₂CHF₂ |
| Me | Cl | H | Me | SO₂Me |
| Me | Cl | H | Me | SO₂CF₃ |
| Me | Cl | H | 3-CF₃-2- | CF₃ |
| | | | pyridyl | |
| Me | Cl | H | CF₃ | Me |
| Me | Cl | H | OMe | CF₃ |
| Me | Cl | H | H | CF₃ |
| Me | Cl | H | H | OCHF₂ |
| Me | Cl | H | H | C₂F₅ |
| Me | H | H | H | CF₃ |
| Me | H | H | H | OCF₃ |
| Et | H | H | H | OCF₃ |
| Me | H | H | Me | Br |
| Me | H | H | Et | Br |
| Me | H | H | Me | Cl |
| Me | H | H | Et | Cl |
| Me | H | H | Me | I |
| Me | H | H | Me | CF₃ |
| Me | H | H | Me | OCF₃ |
| Et | H | H | Me | CF₃ |
| Me | H | H | Me | SCF₃ |
| Me | H | H | Me | SCHF₂ |
| Me | H | H | Me | OCHF₂ |
| n-Pr | H | H | Me | CF₃ |
| Me | H | H | Me | C₂F₅ |
| Et | H | H | Me | C₂F₅ |
| Me | H | H | Et | CF₃ |
| Me | H | H | n-Pr | CF₃ |
| Me | H | H | i-Pr | CF₃ |
| Me | H | H | Cl | CF₃ |
| Me | H | H | F | CF₃ |
| Me | H | H | Me | SMe |
| Me | H | H | Me | OMe |
| Me | H | H | Me | OEt |
| Me | H | H | Me | n-C₃F₇ |
| Me | H | H | Me | i-C₃F₇ |
| Me | H | H | Me | Et |
| Me | H | H | Me | OCF₂CHF₂ |
| Me | H | H | Me | SCF₂CHF₂ |
| Me | H | H | Me | SO₂Me |
| Me | H | H | Me | SO₂CF₃ |
| Me | H | H | CF₃ | CF₃ |
| Me | H | H | CF₃ | Me |
| Me | H | H | OMe | CF₃ |
| Me | H | H | H | CF₃ |
| Me | H | H | H | OCHF₂ |
| Me | H | H | H | C₂F₅ |
| Et | H | H | H | C₂F₅ |
| Me | H | H | H | OCF₃ |
| Me | H | H | H | OCF₂CHF₂ |
| Me | H | H | H | SCF₂CHF₂ |
| Me | H | H | H | n-C₃F₇ |
| Me | H | H | H | i-C₃F₇ |
| Me | H | H | H | Br |
| Me | H | H | H | Cl |
| Me | H | H | H | SCF₃ |
| Me | H | H | Ph | CF₃ |
| Me | H | H | Ph | Cl |
| Me | H | H | Ph | Br |
| Me | H | H | 2-pyridyl | CF₃ |
| Me | H | H | 2-pyridyl | Cl |
| Me | H | H | 2-ClPh | CF₃ |
| Me | H | H | 2-ClPh | OCF₃ |
| Me | H | H | 2-ClPh | Br |
| Me | H | H | 2-ClPh | Cl |
| Me | H | H | 2-ClPh | SCHF₂ |
| Me | H | H | 2-BrPh | CF₃ |
| Me | H | H | 2-MePh | CF₃ |
| Me | H | H | 2-CNPh | CF₃ |
| Me | H | H | 2-FPh | CF₃ |
| Me | H | H | 2,6-F₂Ph | CF₃ |
| Me | H | H | 2,4-F₂Ph | CF₃ |
| Me | H | H | 2,5-F₂Ph | CF₃ |
| Me | H | H | 2-MeOPh | CF₃ |
| Me | H | H | 3-Cl-2-pyridyl | CF₃ |
| Me | H | H | 3-Cl-2-pyridyl | OCF₃ |
| Me | H | H | 3-Cl-2-pyridyl | Br |
| Me | H | H | 3-Cl-2-pyridyl | Cl |
| Me | H | H | 3-Cl-2-pyridyl | SCHF₂ |
| Me | H | H | 3-F-2-pyridyl | CF₃ |
| Me | H | H | 3-CF₃-2- | CF₃ |
| | | | pyridyl | |
| Me | H | H | 3-Me-2- | CF₃ |
| | | | pyridyl | |
| Me | H | H | 3-Br-2-pyridyl | CF₃ |
| Me | H | H | 3-Br-2-pyridyl | OCF₃ |
| Me | H | H | 3-Br-2-pyridyl | Br |
| Me | H | H | 3-Br-2-pyridyl | Cl |
| Me | H | Cl | Et | Br |
| Me | H | Cl | Me | Cl |
| Me | H | Cl | Et | Cl |
| Me | H | Cl | Me | I |
| Me | H | Cl | Me | CF₃ |
| Me | H | Cl | Me | OCF₃ |
| Et | H | Cl | Me | CF₃ |
| Me | H | Cl | Me | SCF₃ |
| Me | H | Cl | Me | SCHF₂ |
| Me | H | Cl | Me | OCHF₂ |
| n-Pr | H | Cl | Me | CF₃ |
| Me | H | Cl | Me | C₂F₅ |
| Et | H | Cl | Me | C₂F₅ |
| Me | H | Cl | Et | CF₃ |
| Me | H | Cl | n-Pr | CF₃ |
| Me | H | Cl | i-Pr | CF₃ |
| Me | H | Cl | Cl | CF₃ |
| Me | H | Cl | F | CF₃ |
| Me | H | Cl | Me | SMe |
| Me | H | Cl | Me | OMe |
| Me | H | Cl | Me | OEt |
| Me | H | Cl | Me | n-C₃F₇ |
| Me | H | Cl | Me | i-C₃F₇ |
| Me | H | Cl | Me | Et |
| Me | H | Cl | Me | OCF₂CHF₂ |
| Me | H | Cl | Me | SCF₂CHF₂ |
| Me | H | Cl | Me | SO₂Me |
| Me | H | Cl | Me | SO₂CF₃ |
| Me | H | Cl | CF₃ | CF₃ |
| Me | H | Cl | CF₃ | Me |
| Me | H | Cl | OMe | CF₃ |
| Me | H | Cl | H | CF₃ |
| Me | H | Cl | H | OCHF₂ |
| Me | H | Cl | H | C₂F₅ |
| Et | H | Cl | H | C₂F₅ |
| Me | H | Cl | H | OCF₃ |
| Me | H | Cl | H | OCF₂CHF₂ |
| Me | H | Cl | H | SCF₂CHF₂ |
| Me | H | Cl | H | n-C₃F₇ |
| Me | H | Cl | H | i-C₃F₇ |
| Me | H | Cl | H | Br |
| Me | H | Cl | H | Cl |
| Me | H | Cl | H | SCF₃ |
| Me | H | Cl | Ph | CF₃ |
| Me | H | Cl | Ph | Cl |
| Me | H | Cl | Ph | Br |
| Me | H | Cl | 2-pyridyl | CF₃ |
| Me | H | Cl | 2-pyridyl | Cl |
| Me | H | Cl | 2-ClPh | CF₃ |
| Me | H | Cl | 2-ClPh | OCF₃ |
| Me | H | Cl | 2-ClPh | Br |
| Me | H | Cl | 2-ClPh | Cl |
| Me | H | Cl | 2-ClPh | SCHF₂ |
| Me | H | Cl | 2-BrPh | CF₃ |
| Me | H | Cl | 2-MePh | CF₃ |
| Me | H | Cl | 2-CNPh | CF₃ |
| Me | H | Cl | 2-FPh | CF₃ |
| Me | H | Cl | 2,6-F₂Ph | CF₃ |
| Me | H | Cl | 2,4-F₂Ph | CF₃ |
| Me | H | Cl | 2,5-F₂Ph | CF₃ |
| Me | H | Cl | 2-MeOPh | CF₃ |
| Me | H | Cl | 3-Cl-2-pyridyl | CF₃ |
| Me | H | Cl | 3-Cl-2-pyridyl | OCF₃ |
| Me | H | Cl | 3-Cl-2-pyridyl | Br |
| Me | H | Cl | 3-Cl-2-pyridyl | Cl |
| Me | H | Cl | 3-Cl-2-pyridyl | SCHF₂ |
| Me | H | Cl | 3-F-2-pyridyl | CF₃ |
| Me | H | Cl | 3-CF₃-2- | CF₃ |
| | | | pyridyl | |
| Me | H | Cl | 3-Me-2- | CF₃ |
| | | | pyridyl | |
| Me | H | Cl | 3-Br-2-pyridyl | CF₃ |
| Me | H | Cl | 3-Br-2-pyridyl | OCF₃ |
| Me | H | Cl | 3-Br-2-pyridyl | Br |
| Me | H | Cl | 3-Br-2-pyridyl | Cl |
| Me | Cl | H | H | OCF₃ |
| Me | Cl | H | H | OCF₂CHF₂ |
| Me | Cl | H | H | SCF₂CHF₂ |
| Me | Cl | H | H | n-C₃F₇ |
| Me | Cl | H | H | i-C₃F₇ |
| Me | Cl | H | H | Br |
| Me | Cl | H | H | Cl |
| Me | Cl | H | H | SCF₃ |
| Me | Cl | H | Ph | CF₃ |
| Me | Cl | H | Ph | Cl |
| Me | Cl | H | Ph | Br |
| Me | Cl | H | 2-pyridyl | CF₃ |
| Me | Cl | H | 2-pyridyl | Cl |
| Me | Cl | H | 2-ClPh | CF₃ |
| Me | Cl | H | 2-ClPh | OCF₃ |
| Me | Cl | H | 2-ClPh | Br |
| Me | Cl | H | 2-ClPh | Cl |
| Me | Cl | H | 2-ClPh | SCHF₂ |
| Me | Cl | H | 2-BrPh | CF₃ |
| Me | Cl | H | 2-MePh | CF₃ |
| Me | Cl | H | 2-CNPh | CF₃ |
| Me | Cl | H | 2-FPh | CF₃ |
| Me | Cl | H | 2,6-F₂Ph | CF₃ |
| Me | Cl | H | 2,4-F₂Ph | CF₃ |
| Me | Cl | H | 2,5-F₂Ph | CF₃ |
| Me | Cl | H | 2-MeOPh | CF₃ |
| Me | Cl | H | 3-Cl-2-pyridyl | CF₃ |
| Me | Cl | H | 3-Cl-2-pyridyl | OCF₃ |
| Me | Cl | H | 3-Cl-2-pyridyl | Br |
| Me | Cl | H | 3-Cl-2-pyridyl | Cl |
| Me | Cl | H | 3-Cl-2-pyridyl | SCHF₂ |
| Me | Cl | H | 3-F-2-pyridyl | CF₃ |
| Me | Cl | H | CF₃ | CF₃ |
| Me | Cl | H | 3-Me-2- | CF₃ |
| | | | pyridyl | |
| Me | Cl | H | 3-Br-2-pyridyl | CF₃ |
| Me | Cl | H | 3-Br-2-pyridyl | OCF₃ |
| Me | Cl | H | 3-Br-2-pyridyl | Br |
| Me | Cl | H | 3-Br-2-pyridyl | Cl |
| Et | Cl | H | H | C₂F₅ |

TABLE 4

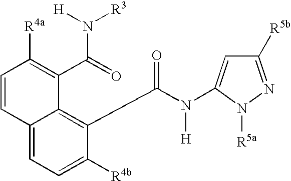

| R³ | R^{4a} | R^{4b} | R^{5a} | R^{5b} |
|---|---|---|---|---|
| Me | H | Me | H | CF₃ |
| Me | H | Me | H | OCF₃ |
| Et | H | Me | H | OCF₃ |
| Me | H | Me | Me | Br |
| Me | H | Me | Et | Br |
| Me | H | Me | Me | Cl |
| Me | H | Me | Et | Cl |
| Me | H | Me | Me | I |
| Me | H | Me | Me | CF₃ |
| Me | H | Me | Me | OCF₃ |
| Et | H | Me | Me | CF₃ |
| Me | H | Me | Me | SCF₃ |
| Me | H | Me | Me | SCHF₂ |
| Me | H | Me | Me | OCHF₂ |
| n-Pr | H | Me | Me | CF₃ |
| Me | H | Me | Me | C₂F₅ |
| Et | H | Me | Me | C₂F₅ |
| Me | H | Me | Et | CF₃ |
| Me | H | Me | n-Pr | CF₃ |
| Me | H | Me | i-Pr | CF₃ |
| Me | H | Mc | i-Pr | OCF₃ |
| Me | H | Me | Me | SMe |
| Me | H | Me | Me | OMe |
| Me | H | Me | Me | OEt |
| Me | H | Me | Me | n-C₃F₇ |
| Me | H | Me | Me | i-C₃F₇ |
| Me | H | Me | Me | Et |
| Me | H | Me | Me | OCF₂CHF₂ |
| Me | H | Me | Me | SCF₂CHF₂ |
| Me | H | Me | Me | SO₂Me |
| Me | H | Me | Me | SO₂CF₃ |
| Me | H | Me | CHF₂ | CF₃ |
| Me | H | Me | CHF₂ | Me |
| Me | H | Me | Ph | CF₃ |
| Me | H | Me | Ph | Cl |
| Me | H | Me | Ph | Br |
| Me | H | Me | 2-pyridyl | CF₃ |
| Me | H | Me | 2-pyridyl | Cl |
| Me | H | Me | 2-ClPh | CF₃ |
| Me | H | Me | 2-ClPh | OCF₃ |
| Me | H | Me | 2-ClPh | Br |
| Me | H | Me | 2-ClPh | Cl |
| Me | H | Me | 2-ClPh | SCHF₂ |
| Me | H | Me | 2-BrPh | CF₃ |
| Me | H | Me | 2-MePh | CF₃ |
| Me | H | Me | 2-CNPh | CF₃ |
| Me | H | Me | 2-FPh | CF₃ |
| Me | H | Me | 2,6-F₂Ph | CF₃ |
| Me | H | Me | 2,4-F₂Ph | CF₃ |
| Me | H | Me | 2,5-F₂Ph | CF₃ |
| Me | H | Me | 2-MeOPh | CF₃ |
| Me | H | Me | 3-Cl-2-pyridyl | CF₃ |
| Me | H | Me | 3-Cl-2-pyridyl | OCF₃ |
| Me | H | Me | 3-Cl-2-pyridyl | Br |
| Me | H | Me | 3-Cl-2-pyridyl | Cl |
| Me | H | Me | 3-Cl-2-pyridyl | SCHF₂ |
| Me | H | Me | 3-CF₃-2- | CF₃ |
| | | | pyridyl | |
| Me | H | Me | 3-Me-2- | CF₃ |
| | | | pyridyl | |
| Me | H | Me | 3-F-2-pyridyl | CF₃ |
| Me | H | Me | 3-Br-2-pyridyl | CF₃ |
| Me | H | Me | 3-Br-2-pyridyl | OCF₃ |
| Me | H | Me | 3-Br-2-pyridyl | Br |
| Me | H | Me | 3-Br-2-pyridyl | Cl |
| Me | Me | H | Me | Br |
| Me | Me | H | Et | Br |
| Me | Me | H | Me | Cl |
| Me | Me | H | Et | Cl |
| Me | Me | H | Me | I |
| Me | Me | H | Me | CF₃ |
| Me | Me | H | Me | OCF₃ |
| Et | Me | H | Me | CF₃ |
| Me | Me | H | Me | SCF₃ |
| Me | Me | H | Me | SCHF₂ |
| Me | Me | H | Me | OCHF₂ |
| n-Pr | Me | H | Me | CF₃ |
| Me | Me | H | Me | C₂F₅ |
| Et | Me | H | Me | C₂F₅ |
| Me | Me | H | Et | CF₃ |
| Me | Me | H | n-Pr | CF₃ |
| Me | Me | H | i-Pr | CF₃ |
| Me | Me | H | Me | SMe |
| Me | Me | H | Me | OMe |
| Me | Me | H | Me | OEt |
| Me | Me | H | Me | n-C₃F₇ |
| Me | Me | H | Me | i-C₃F₇ |
| Me | Me | H | Me | Et |
| Me | Me | H | Me | OCF₂CHF₂ |
| Me | Me | H | Me | SCF₂CHF₂ |
| Me | Me | H | Me | SO₂Me |
| Me | Me | H | Me | SO₂CF₃ |
| Me | Me | H | CF₃ | CF₃ |
| Me | Me | H | CF₃ | Me |
| Me | Me | H | Ph | CF₃ |
| Me | Me | H | Ph | Cl |
| Me | Me | H | Ph | Br |
| Me | Me | H | 2-pyridyl | CF₃ |
| Me | Me | H | 2-pyridyl | Cl |
| Me | Me | H | 2-ClPh | CF₃ |
| Me | Me | H | 2-ClPh | OCF₃ |
| Me | Me | H | 2-ClPh | Br |
| Me | Me | H | 2-ClPh | Cl |
| Me | Me | H | 2-ClPh | SCHF₂ |
| Me | Me | H | 2-BrPh | CF₃ |
| Me | Me | H | 2-MePh | CF₃ |
| Me | Me | H | 2-CNPh | CF₃ |
| Me | Me | H | 2-FPh | CF₃ |
| Me | Me | H | 2,6-F₂Ph | CF₃ |
| Me | Me | H | 2,4-F₂Ph | CF₃ |
| Me | Me | H | 2,5-F₂Ph | CF₃ |
| Me | Me | H | 2-MeOPh | CF₃ |
| Me | Me | H | 3-Cl-2-pyridyl | CF₃ |
| Me | Me | H | 3-Cl-2-pyridyl | OCF₃ |
| Me | Me | H | 3-Cl-2-pyridyl | Br |
| Me | Me | H | 3-Cl-2-pyridyl | Cl |
| Me | Me | H | 3-CF₃-2- | CF₃ |
| | | | pyridyl | |
| Me | Me | H | 3-Me-2- | CF₃ |
| | | | pyridyl | |
| Me | Me | H | 3-Cl-2-pyridyl | SHF₂ |
| Me | Me | H | 3-F-2-pyridyl | CF₃ |
| Me | Me | H | 3-Br-2-pyridyl | CF₃ |
| Me | Me | H | 3-Br-2-pyridyl | OCF₃ |
| Me | Me | H | 3-Br-2-pyridyl | Br |
| Me | Me | H | 3-Br-2-pyridyl | Cl |
| Me | Cl | H | Me | Br |
| Me | Cl | H | Et | Br |
| Me | Cl | H | Me | Cl |
| Me | Cl | H | Et | Cl |
| Me | Cl | H | Me | I |
| Me | Cl | H | Me | CF₃ |
| Me | Cl | H | Me | OCF₃ |
| Et | Cl | H | Me | CF₃ |
| Me | Cl | H | Me | SCF₃ |
| Me | Cl | H | Me | SCHF₂ |
| Me | Cl | H | Me | OCHF₂ |
| n-Pr | Cl | H | Me | CF₃ |
| Me | Cl | H | Me | C₂F₅ |
| Et | Cl | H | Me | C₂F₅ |
| Me | Cl | H | 3-Cl-2-pyridyl | CF₃ |
| Me | Cl | H | 3-Cl-2-pyridyl | OCF₃ |
| Me | Cl | H | 3-Cl-2-pyridyl | Br |
| Me | Cl | H | 3-Cl-2-pyridyl | Cl |
| Me | Cl | H | 3-Cl-2-pyridyl | SHF₂ |
| Me | Cl | H | 3-F-2-pyridyl | CF₃ |
| Me | Cl | H | 3-CF₃-2- | CF₃ |
| | | | pyridyl | |
| Me | Cl | H | 3-Br-2-pyridyl | CF₃ |
| Me | Cl | H | 3-Br-2-pyridyl | OCF₃ |
| Me | Cl | H | 3-Br-2-pyridyl | Br |
| Me | Cl | H | 3-Br-2-pyridyl | Cl |
| Me | Cl | H | 2,4-F₂Ph | CF₃ |
| Me | Cl | H | 2,5-F₂Ph | CF₃ |
| Me | Cl | H | 2-OMe | CF₃ |
| Me | Cl | H | 2-ClPh | Br |
| Me | H | H | H | CF₃ |
| Me | H | H | H | OCF₃ |
| Et | H | H | H | OCF₃ |
| Me | H | H | Me | Br |
| Me | H | H | Et | Br |
| Me | H | H | Me | CI |
| Me | H | H | Et | Cl |
| Me | H | H | Me | I |
| Me | H | H | Me | CF₃ |
| Me | H | H | Me | OCF₃ |
| Et | H | H | Me | CF₃ |
| Me | H | H | Me | SCF₃ |
| Me | H | H | Me | SCHF₂ |
| Me | H | H | Me | OCHF₂ |
| n-Pr | H | H | Me | CF₃ |
| Me | H | H | Me | C₂F₅ |
| Et | H | H | Me | C₂F₅ |
| Me | H | H | Et | CF₃ |
| Me | H | H | n-Pr | CF₃ |
| Me | H | H | i-Pr | CF₃ |
| Me | H | H | Me | SMe |
| Me | H | H | Me | OMe |
| Me | H | H | Me | OEt |
| Me | H | H | Me | n-C₃F₇ |
| Me | H | H | Me | i-C₃F₇ |
| Me | H | H | Me | Et |
| Me | H | H | Me | OCF₂CHF₂ |
| Me | H | H | Me | SCF₂CHF₂ |
| Me | H | H | Me | SO₂Me |
| Me | H | H | Me | SO₂CF₃ |
| Me | H | H | CF₃ | CF₃ |
| Me | H | H | CF₃ | Me |
| Me | H | H | Ph | CF₃ |
| Me | H | H | Ph | Cl |
| Me | H | H | Ph | Br |
| Me | H | H | 2-pyridyl | CF₃ |
| Me | H | H | 2-pyridyl | Cl |
| Me | H | H | 2-ClPh | CF₃ |
| Me | H | H | 2-ClPh | OCF₃ |
| Me | H | H | 2-ClPh | Br |
| Me | H | H | 2-ClPh | Cl |
| Me | H | H | 2-ClPh | SCHF₂ |
| Me | H | H | 2-BrPh | CF₃ |
| Me | H | H | 2-MePh | CF₃ |
| Me | H | H | 2-CNPh | CF₃ |
| Me | H | H | 2-FPh | CF₃ |
| Me | H | H | 2,6-F₂Ph | CF₃ |
| Me | H | H | 2,4-F₂Ph | CF₃ |
| Me | H | H | 2,5-F₂Ph | CF₃ |
| Me | H | H | 2-MeOPh | CF₃ |
| Me | H | H | 3-Cl-2-pyridyl | CF₃ |
| Me | H | H | 3-Cl-2-pyridyl | OCF₃ |
| Me | H | H | 3-Cl-2-pyridyl | Br |
| Me | H | H | 3-Cl-2-pyridyl | Cl |
| Me | H | H | 3-Cl-2-pyridyl | SCHF₂ |
| Me | H | H | 3-F-2-pyridyl | CF₃ |
| Me | H | H | 3-CF₃-2- | CF₃ |
| | | | pyridyl | |
| Me | H | H | 3-Me-2- | CF₃ |
| | | | pyridyl | |
| Me | H | H | 3-Br-2-pyridyl | CF₃ |
| Me | H | H | 3-Br-2-pyridyl | OCF₃ |
| Me | H | H | 3-Br-2-pyridyl | Br |
| Me | H | H | 3-Br-2-pyridyl | Cl |
| Me | H | Cl | Et | Br |
| Me | H | Cl | Me | Cl |
| Me | H | Cl | Et | Cl |
| Me | H | Cl | Me | I |
| Me | H | Cl | Me | CF₃ |
| Me | H | Cl | Me | OCF₃ |
| Et | H | Cl | Me | CF₃ |
| Me | H | Cl | Me | SCF₃ |
| Me | H | Cl | Me | SCHF₂ |
| Me | H | Cl | Me | OCHF₂ |
| n-Pr | H | Cl | Me | CF₃ |
| Me | H | Cl | Me | C₂F₅ |
| Et | H | Cl | Me | C₂F₅ |
| Me | H | Cl | Et | CF₃ |
| Me | H | Cl | n-Pr | CF₃ |
| Me | H | Cl | i-Pr | CF₃ |
| Me | H | Cl | Me | SMe |
| Me | H | Cl | Me | OMe |
| Me | H | Cl | Me | OEt |
| Me | H | Cl | Me | n-C₃F₇ |
| Me | H | Cl | Me | i-C₃F₇ |
| Me | H | Cl | Me | Et |
| Me | H | Cl | Me | OCF₂CHF₂ |
| Me | H | Cl | Me | SCF₂CHF₂ |
| Me | H | Cl | Me | SO₂Me |
| Me | H | Cl | Me | SO₂CF₃ |
| Me | H | Cl | CF₃ | CF₃ |
| Me | H | Cl | CF₃ | Me |
| Me | H | Cl | Ph | CF₃ |
| Me | H | Cl | Ph | Cl |
| Me | H | Cl | Ph | Br |
| Me | H | Cl | 2-pyridyl | CF₃ |
| Me | H | Cl | 2-pyridyl | Cl |
| Me | H | Cl | 2-ClPh | CF₃ |
| Me | H | Cl | 2-ClPh | OCF₃ |
| Me | H | Cl | 2-ClPh | Br |
| Me | H | Cl | 2-ClPh | Cl |
| Me | H | Cl | 2-ClPh | SCHF₂ |
| Me | H | Cl | 2-BrPh | CF₃ |
| Me | H | Cl | 2-MePh | CF₃ |
| Me | H | Cl | 2-CNPh | CF₃ |
| Me | H | Cl | 2-FPh | CF₃ |
| Me | H | Cl | 2,6-F₂Ph | CF₃ |
| Me | H | Cl | 2,4-F₂Ph | CF₃ |
| Me | H | Cl | 2,5-F₂Ph | CF₃ |
| Me | H | Cl | 2-OMe | CF₃ |
| Me | H | Cl | 3-Cl-2-pyridyl | CF₃ |
| Me | H | Cl | 3-Cl-2-pyridyl | OCF₃ |
| Me | H | Cl | 3-Cl-2-pyridyl | Br |
| Me | H | Cl | 3-Cl-2-pyridyl | Cl |
| Me | H | Cl | 3-Cl-2-pyridyl | SCHF₂ |
| Me | H | Cl | 3-F-2-pyridyl | CF₃ |
| Me | H | Cl | 3-CF₃-2- | CF₃ |
| | | | pyridyl | |
| Me | H | Cl | 3-Me-2- | CF₃ |
| | | | pyridyl | |
| Me | H | Cl | 3-Br-2-pyridyl | CF₃ |
| Me | H | Cl | 3-Br-2-pyridyl | OCF₃ |
| Me | H | Cl | 3-Br-2-pyridyl | Br |
| Me | H | Cl | 3-Br-2-pyridyl | Cl |
| Me | Cl | H | Et | CF₃ |
| Me | Cl | H | n-Pr | CF₃ |
| Me | Cl | H | i-Pr | CF₃ |
| Me | Cl | H | Me | SMe |
| Me | Cl | H | Me | OMe |
| Me | Cl | H | Me | OEt |
| Me | Cl | H | Me | n-C₃F₇ |
| Me | Cl | H | Me | i-C₃F₇ |
| Me | Cl | H | Me | Et |
| Me | Cl | H | Me | OCF₂CHF₂ |
| Me | Cl | H | Me | SCF₂CHF₂ |
| Me | Cl | H | Me | SO₂Me |
| Me | Cl | H | Me | SO₂CF₃ |
| Me | Cl | H | CF₃ | CF₃ |
| Me | Cl | H | CF₃ | Me |
| Me | Cl | H | OMe | CF₃ |
| Me | Cl | H | Ph | CF₃ |
| Me | Cl | H | Ph | Cl |
| Me | Cl | H | Ph | Br |
| Me | Cl | H | 2-pyridyl | CF₃ |
| Me | Cl | H | 2-pyridyl | Cl |
| Me | Cl | H | 2-ClPh | CF₃ |
| Me | Cl | H | 3-Me-2- | CF₃ |
| | | | pyridyl | |
| Me | Cl | H | 2-ClPh | Cl |
| Me | Cl | H | 2-ClPh | SCHF₂ |
| Me | Cl | H | 2-BrPh | CF₃ |
| Me | Cl | H | 2-MePh | CF₃ |
| Me | Cl | H | 2-CNPh | CF₃ |
| Me | Cl | H | 2-FPh | CF₃ |
| Me | Cl | H | 2,6-F₂Ph | CF₃ |
| Me | Cl | H | 2-ClPh | OCF-₃ |

TABLE 5

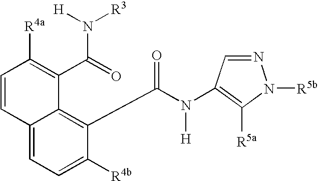

| R³ | R^{4a} | R^{4b} | R^{5a} | R^{5b} |
|---|---|---|---|---|
| Me | H | Me | H | CHF₂ |
| Me | H | Me | H | CH₂CF₃ |
| Et | H | Me | H | CH₂CF₃ |
| Me | H | Me | Me | CH₂CF₃ |
| Me | H | Me | Et | CH₂CF₃ |
| Me | H | Me | Me | CF₂CHF₂ |
| Me | H | Me | Et | CHF₂ |
| Me | H | Me | Me | CHF₂ |
| Me | H | Me | Me | CBrF₂ |
| Me | H | Me | Me | CHF₂ |
| Et | H | Me | Me | CH₂CF₃ |
| Me | H | Me | Me | Et |
| Me | H | Me | Me | n-Pr |
| Me | H | Me | Me | CH₂C₂F₅ |
| n-Pr | H | Me | Me | CH₂CF₃ |
| Me | H | Me | Me | CF₃ |
| Et | H | Me | Me | C₂F₅ |
| Me | H | Me | Et | CHF₂ |
| Me | H | Me | n-Pr | CH₂CF₃ |
| Me | H | Me | i-Pr | CHF₂ |
| Me | H | Me | Cl | CH₂CF₃ |
| Me | H | Me | F | CH₂CF₃ |
| Me | H | Me | Me | CH₂Cl |
| Me | H | Me | Me | CClF₂ |
| Me | H | Me | Me | CH₂CH₂Cl |
| Me | H | Me | Me | n-C₃F₇ |
| Me | H | Me | Me | i-C₃F₇ |
| Me | H | Me | Me | Allyl |
| Me | H | Me | Et | CF₂CHF₂ |
| Me | H | Me | Et | i-C₃F₇ |
| Me | H | Me | i-Pr | CF₂CHF₂ |
| Me | H | Me | n-Pr | CF₂CHF₂ |
| Me | H | Me | CF₃ | CF₂CHF₂ |
| Me | H | Me | CF₃ | Me |
| Me | H | Me | OMe | CH₂CF₃ |
| Me | H | Me | H | CH₂CF₃ |
| Me | H | Me | H | CH₂CF₃ |
| Me | H | Me | H | C₂F₅ |
| Et | H | Me | H | C₂F₅ |
| Me | H | Me | H | C₂F₅ |
| Me | H | Me | H | CF₂CHF₂ |
| Me | H | Me | i-Pr | CH₂CF₃ |
| Me | H | Me | H | n-C₃F₇ |
| Me | H | Me | H | i-C₃F₇ |
| Me | H | Me | Ph | CH₂CF₃ |
| Me | H | Me | Ph | CF₂CHF₂ |
| Me | H | Me | Ph | CHF₂ |
| Me | H | Me | 2-pyridyl | CH₂CF₃ |
| Me | H | Me | 2-pyridyl | CF₂CHF₂ |
| Me | H | Me | 2-ClPh | CH₂CF₃ |
| Me | H | Me | 2-ClPh | CF₂CHF₂ |
| Me | H | Me | 2-ClPh | CHF₂ |
| Me | H | Me | 2-ClPh | Et |
| Me | H | Me | 2-ClPh | CBrF₂ |
| Me | H | Me | 2-BrPh | CH₂CF₃ |
| Me | H | Me | 2-MePh | CH₂CF₃ |
| Me | H | Me | 2-CNPh | CH₂CF₃ |
| Me | H | Me | 2-FPh | CH₂CF₃ |
| Me | H | Me | 2,6-F₂Ph | CH₂CF₃ |
| Me | H | Me | 2,4-F₂Ph | CH₂CF₃ |
| Me | H | Me | 2,5-F₂Ph | CH₂CF₃ |
| Me | H | Me | 2-MeOPh | CH₂CF₃ |
| Me | H | Me | 3-Cl-2-pyridyl | CH₂CF₃ |
| Me | H | Me | 3-Cl-2-pyridyl | CF₂CHF₂ |
| Me | H | Me | 3-Cl-2-pyridyl | CF₃ |
| Me | H | Me | 3-Cl-2-pyridyl | CHF₂ |
| Me | H | Me | 3-Cl-2-pyridyl | CBrF₂ |
| Me | H | Me | 3-F-2-pyridyl | CH₂CF₃ |
| Me | H | Me | 3-CF₃-2- | CH₂CF₃ |
| | | | pyridyl | |
| Me | H | Me | 3-Me-2- | CH₂CF₃ |
| | | | pyridyl | |
| Me | H | Me | 3-Br-2-pyridyl | CF₃ |
| Me | H | Me | 3-Br-2-pyridyl | CH₂CF₃ |
| Me | H | Me | 3-Br-2-pyridyl | CF₂CHF₂ |
| Me | H | Me | 3-Br-2-pyridyl | CClF₂ |
| Me | Me | H | H | CHF₂ |
| Me | Me | H | H | CH₂CF₃ |
| Et | Me | H | H | CH₂CF₃ |
| Me | Me | H | Me | CH₂CF₃ |
| Me | Me | H | Et | CH₂CF₃ |
| Me | Me | H | Me | CF₂CHF₂ |
| Me | Me | H | Et | CHF₂ |
| Me | Me | H | Me | CHF₂ |
| Me | Me | H | Me | CBrF₂ |
| Me | Me | H | Me | CHF₂ |
| Et | Me | H | Me | CH₂CF₃ |
| Me | Me | H | Me | Et |
| Me | Me | H | Me | n-Pr |
| Me | Me | H | Me | CH₂C₂F₅ |
| n-Pr | Me | H | Me | CH₂CF₃ |
| Me | Me | H | Me | CF₃ |
| Et | Me | H | Me | C₂F₅ |
| Me | Me | H | Et | CHF₂ |
| Me | Me | H | n-Pr | CH₂CF₃ |
| Me | Me | H | i-Pr | CHF₂ |
| Me | Me | H | Cl | CH₂CF₃ |
| Me | Me | H | F | CH₂CF₃ |
| Me | Me | H | Me | CH₂Cl |
| Me | Me | H | Me | CClF₂ |
| Me | Me | H | Me | CH₂CH₂Cl |
| Me | Me | H | Me | n-C₃F₇ |
| Me | Me | H | Me | i-C₃F₇ |
| Me | Me | H | Me | Allyl |
| Me | Me | H | Me | CF₂CHF₂ |
| Me | Me | H | Me | i-C₃F₇ |
| Me | Me | H | Me | CF₂CHF₂ |
| Me | Me | H | Me | CF₂CHF₂ |
| Me | Me | H | CF₃ | CF₂CHF₂ |
| Me | Me | H | CF₃ | Me |
| Me | Me | H | OMe | CH₂CF₃ |
| Me | Me | H | H | CH₂CF₃ |
| Me | Me | H | H | CH₂CF₃ |
| Me | Me | H | H | C₂F₅ |
| Et | Me | H | H | C₂F₅ |
| Me | Me | H | H | C₂F₅ |
| Me | Me | H | H | CF₂CHF₂ |
| Me | Me | H | H | CH₂CF₃ |
| Me | Me | H | H | n-C₃F₇ |
| Me | Me | H | H | i-C₃F₇ |
| Me | Me | H | H | CH₂CF₃ |
| Me | Me | H | H | CF₂CHF₂ |
| Me | Me | H | H | CHF₂ |
| Me | Me | H | Ph | CH₂CF₃ |
| Me | Me | H | Ph | CF₂CHF₂ |
| Me | Me | H | Ph | CH₂CF₃ |
| Me | Me | H | 2-pyridyl | CF₂CHF₂ |
| Me | Me | H | 2-pyridyl | CHF₂ |
| Me | Me | H | 2-ClPh | Et |
| Me | Me | H | 2-ClPh | CBrF₂ |
| Me | Me | H | 2-ClPh | CH₂CF₃ |
| Me | Me | H | 2-ClPh | CH₂CF₃ |
| Me | Me | H | 2-ClPh | CH₂CF₃ |
| Me | Me | H | 2-BrPh | CH₂CF₃ |
| Me | Me | H | 2-MePh | CH₂CF₃ |
| Me | Me | H | 2-CNPh | CH₂CF₃ |
| Me | Me | H | 2-FPh | CH₂CF₃ |
| Me | Me | H | 2,6-F₂Ph | CH₂CF₃ |
| Me | Me | H | 2,4-F₂Ph | CH₂CF₃ |
| Me | Me | H | 2,5-F₂Ph | CF₂CHF₂ |
| Me | Me | H | 2-MeOPh | CF₃ |
| Me | Me | H | 3-Cl-2-pyridyl | CHF₂ |
| Me | Me | H | 3-Cl-2-pyridyl | CBrF₂ |
| Me | Me | H | 3-Cl-2-pyridyl | CH₂CF₃ |
| Me | Me | H | 3-Cl-2-pyridyl | CH₂CF₃ |
| Me | Me | H | 3-Cl-2-pyridyl | CH₂CF₃ |
| Me | Me | H | 3-F-2-pyridyl | CF₃ |
| Me | Me | H | 3-CF₃-2- | CH₂CF₃ |
| | | | pyridyl | |
| Me | Me | H | 3-Me-2- | CF₂CHF₂ |
| | | | pyridyl | |
| Me | Me | H | 3-Br-2-pyridyl | CClF₂ |
| Me | Me | H | 3-Br-2-pyridyl | CH₂CF₃ |
| Me | Me | H | 3-Br-2-pyridyl | CF₃ |
| Me | Me | H | 3-Br-2-pyridyl | CF₃ |
| Me | Cl | H | Me | CHF₂ |
| Me | Cl | H | Et | CH₂CF₃ |
| Me | Cl | H | Me | CH₂CF₃ |
| Me | Cl | H | Et | CH₂CF₃ |
| Me | Cl | H | Me | CH₂CF₃ |
| Me | Cl | H | Me | CF₂CHF₂ |
| Me | Cl | H | Me | CHF₂ |
| Et | Cl | H | Me | CHF₂ |
| Me | Cl | H | Me | CBrF₂ |
| Me | Cl | H | Me | CHF₂ |
| Me | Cl | H | Me | CH₂CF₃ |
| n-Pr | Cl | H | Me | Et |
| Me | Cl | H | Me | n-Pr |
| Et | Cl | H | Me | CH₂C₂F₅ |
| Me | Cl | H | Et | CH₂CF₃ |
| Me | Cl | H | n-Pr | CF₃ |
| Me | Cl | H | i-Pr | C₂F₅ |
| Me | Cl | H | Cl | CHF₂ |
| Me | Cl | H | 3-Cl-2-pyridyl | CH₂CF₃ |
| Me | Cl | H | 3-Cl-2-pyridyl | CF₂CHF₂ |
| Me | Cl | H | 3-Cl-2-pyridyl | CF₃ |
| Me | Cl | H | 3-Cl-2-pyridyl | CHF₂ |
| Me | Cl | H | 3-Cl-2-pyridyl | CBrF₂ |
| Me | Cl | H | 3-F-2-pyridyl | CH₂CF₃ |
| Me | Cl | H | 3-CF₃-2- | CH₂CF₃ |
| | | | pyridyl | |
| Me | Cl | H | 3-Br-2-pyridyl | CF₃ |
| Me | Cl | H | 3-Br-2-pyridyl | CH₂CF₃ |
| Me | Cl | H | 3-Br-2-pyridyl | CF₂CHF₂ |
| Me | Cl | H | 3-Br-2-pyridyl | CCF₂ |
| Me | Cl | H | 2-ClPh | CHF₂ |
| Me | Cl | H | 2-ClPh | Et |
| Me | Cl | H | 2-ClPh | CBrF₂ |
| Me | Cl | H | 2-BrPh | CH₂CF₃ |
| Me | Cl | H | 2-MePh | CH₂CF₃ |
| Me | Cl | H | 2-CNPh | CH₂CF₃ |
| Me | Cl | H | 2-MeOPh | CH₂CF₃ |
| Me | Cl | H | 2,5-F₂Ph | CH₂CF₃ |
| Me | H | H | H | CH₂ |
| Me | H | H | H | CH₂CF₃ |
| Et | H | H | H | CH₂CF₃ |
| Me | H | H | Me | CH₂CF₃ |
| Me | H | H | Et | CH₂CF₃ |
| Me | H | H | Me | CF₂CHF₂ |
| Me | H | H | Et | CHF₂ |
| Me | H | H | Me | CHF₂ |
| Me | H | H | Me | CBrF₂ |
| Me | H | H | Me | CHF₂ |
| Et | H | H | Me | CH₂CF₃ |
| Me | H | H | Me | Et |
| Me | H | H | Me | n-Pr |
| Me | H | H | Me | CH₂C₂F₅ |
| n-Pr | H | H | Me | CH₂CF₃ |
| Me | H | H | Me | CF₃ |
| Et | H | H | Me | C₂F₅ |
| Me | H | H | Et | CHF₂ |
| Me | H | H | n-Pr | CH₂CF₃ |
| Me | H | H | i-Pr | CHF₂ |
| Me | H | H | Cl | CH₂CF₃ |
| Me | H | H | F | CH₂CF₃ |
| Me | H | H | Me | CH₂Cl |
| Me | H | H | Me | CClF₂ |
| Me | H | H | Me | CH₂CH₂Cl |
| Me | H | H | Me | n-C₃F₇ |
| Me | H | H | Me | i-C₃F₇ |
| Me | H | H | Me | Allyl |
| Me | H | H | Me | CF₂CHF₂ |
| Me | H | H | Me | i-C₃F₇ |
| Me | H | H | Me | CF₂CHF₂ |
| Me | H | H | Me | CF₂CHF₂ |
| Me | H | H | CF₃ | CF₂CHF₂ |
| Me | H | H | CF₃ | Me |
| Me | H | H | OMe | CH₂CF₃ |
| Me | H | H | H | CH₂CF₃ |
| Me | H | H | H | CH₂CF₃ |
| Me | H | H | H | C₂F₅ |
| Et | H | H | H | C₂F₅ |
| Me | H | H | H | C₂F₅ |
| Me | H | H | H | CF₂CHF₂ |
| Me | H | H | H | CH₂CF₃ |
| Me | H | H | H | n-C₃F₇ |
| Me | H | H | H | i-C₃F₇ |
| Me | H | H | H | CH₂CF₃ |
| Me | H | H | H | CF₂CHF₂ |
| Me | H | H | H | CHF₂ |
| Me | H | H | Ph | CH₂CF₃ |
| Me | H | H | Ph | CF₂CHF₂ |
| Me | H | H | Ph | CH₂CF₃ |
| Me | H | H | 2-pyridyl | CF₂CHF₂ |
| Me | H | H | 2-pyridyl | CHF₂ |
| Me | H | H | 2-ClPh | Et |
| Me | H | H | 2-ClPh | CBrF₂ |
| Me | H | H | 2-ClPh | CH₂CF₃ |
| Me | H | H | 2-ClPh | CH₂CF₃ |
| Me | H | H | 2-ClPh | CH₂CF₃ |
| Me | H | H | 2-BrPh | CH₂CF₃ |
| Me | H | H | 2-MePh | CH₂CF₃ |
| Me | H | H | 2-CNPh | CH₂CF₃ |
| Me | H | H | 2-FPh | CH₂CF₃ |
| Me | H | H | 2,6-F₂Ph | CH₂CF₃ |
| Me | H | H | 2,4-F₂Ph | CH₂CF₃ |
| Me | H | H | 2,5-F₂Ph | CF₂CHF₂ |
| Me | H | H | 2-MeOPh | CF₃ |
| Me | H | H | 3-Cl-2-pyridyl | CHF₂ |
| Me | H | H | 3-Cl-2-pyridyl | CBrF₂ |
| Me | H | H | 3-Cl-2-pyridyl | CH₂CF₃ |
| Me | H | H | 3-CF₃-2- | CH₂CF₃ |
| | | | pyridyl | |
| Me | H | H | 3-Me-2- | CF₂CHF₂ |
| | | | pyridyl | |
| Me | H | H | 3-F-2-pyridyl | CF₃ |
| Me | H | H | 3-Cl-2-pyridyl | CH₂CF₃ |
| Me | H | H | 3-Cl-2-pyridyl | CH₂CF₃ |
| Me | H | H | 3-Br-2-pyridyl | CClF₂ |
| Me | H | H | 3-Br-2-pyridyl | CH₂CF₃ |
| Me | H | H | 3-Br-2-pyridyl | CF₃ |
| Me | H | H | 3-Br-2-pyridyl | CF₃ |
| Me | H | Cl | Et | CHF₂ |
| Me | H | Cl | Me | CH₂CF₃ |
| Me | H | Cl | Et | CH₂CF₃ |
| Me | H | Cl | Me | CH₂CF₃ |
| Me | H | Cl | Me | CH₂CF₃ |
| Me | H | Cl | Me | CF₂CHF₂ |
| Et | H | Cl | Me | CHF₂ |
| Me | H | Cl | Me | CHF₂ |
| Me | H | Cl | Me | CBrF₂ |
| Me | H | Cl | Me | CHF₂ |
| n-Pr | H | Cl | Me | CH₂CF₃ |
| Me | H | Cl | Me | Et |
| Et | H | Cl | Me | n-Pr |
| Me | H | Cl | Et | CH₂C₂F₅ |
| Me | H | Cl | n-Pr | CH₂CF₃ |
| Me | H | Cl | i-Pr | CF₃ |
| Me | H | Cl | Cl | C₂F₅ |
| Me | H | Cl | F | CHF₂ |
| Me | H | Cl | Me | CH₂CF₃ |
| Me | H | Cl | Me | CHF₂ |
| Me | H | Cl | Me | CH₂CF₃ |
| Me | H | Cl | Me | CH₂CF₃ |
| Me | H | Cl | Me | CH₂Cl |
| Me | H | Cl | Me | CClF₂ |
| Me | H | Cl | Me | CH₂CH₂Cl |
| Me | H | Cl | Me | n-C₃F₇ |
| Me | H | Cl | Me | i-C₃F₇ |
| Me | H | Cl | Me | Allyl |
| Me | H | Cl | CF₃ | CF₂CHF₂ |
| Me | H | Cl | CF₃ | i-C₃F₇ |
| Me | H | Cl | OMe | CF₂CHF₂ |
| Me | H | Cl | H | CF₂CHF₂ |
| Me | H | Cl | H | CF₂CHF₂ |
| Me | H | Cl | H | Me |
| Et | H | Cl | H | CH₂CF₃ |
| Me | H | Cl | H | CH₂CF₃ |
| Me | H | Cl | H | CH₂CF₃ |
| Me | H | Cl | H | C₂F₅ |
| Me | H | Cl | H | C₂F₅ |
| Me | H | Cl | H | C₂F₅ |
| Me | H | Cl | H | CF₂CHF₂ |
| Me | H | Cl | H | CH₂CF₃ |
| Me | H | Cl | H | n-C₃F₇ |
| Me | H | Cl | Ph | i-C₃F₇ |
| Me | H | Cl | Ph | CH₂CF₃ |
| Me | H | Cl | Ph | CF₂CHF₂ |
| Me | H | Cl | 2-pyridyl | CHF₂ |
| Me | H | Cl | 2-pyridyl | CH₂CF₃ |
| Me | H | Cl | 2-ClPh | CF₂CHF₂ |
| Me | H | Cl | 2-ClPh | CH₂CF₃ |
| Me | H | Cl | 2-ClPh | CF₂CHF₂ |
| Me | H | Cl | 2-ClPh | CHF₂ |
| Me | H | Cl | 2-ClPh | Et |
| Me | H | Cl | 2-BrPh | CBrF₂ |
| Me | H | Cl | 2-MePh | CH₂CF₃ |
| Me | H | Cl | 2-CNPh | CH₂CF₃ |
| Me | H | Cl | 2-FPh | CH₂CF₃ |
| Me | H | Cl | 2,6-F₂Ph | CH₂CF₃ |
| Me | H | Cl | 2,4-F₂Ph | CH₂CF₃ |
| Me | H | Cl | 2,5-F₂Ph | CH₂CF₃ |
| Me | H | Cl | 2-MeOPh | CH₂CF₃ |
| Me | H | CL | 3-Cl-2-pyridyl | CH₂CF₃ |
| Me | H | Cl | 3-Cl-2-pyridyl | CH₂CF₃ |
| Me | H | Cl | 3-Cl-2-pyridyl | CF₂CHF₂ |
| Me | H | Cl | 3-Cl-2-pyridyl | CF₃ |
| Me | H | Cl | 3-Cl-2-pyridyl | CHF₂ |
| Me | H | Cl | 3-F-2-pyridyl | CBrF₂ |
| Me | H | CL | 3-Br-2-pyridyl | CH₂CF₃ |
| Me | H | Cl | 3-CF₃-2- | CH₂CF₃ |
| | | | pyridyl | |
| Me | H | Cl | 3-Me-2- | CH₂CF₃ |
| | | | pyridyl | |
| Me | H | Cl | 3-Br-2-pyridyl | CF₃ |
| Me | H | Cl | 3-Br-2-pyridyl | CH₂CF₃ |
| Me | H | Cl | 3-Br-2-pyridyl | CF₂CHF₂ |
| Me | Cl | H | F | Cl₂CF₃ |
| Me | Cl | H | Me | CHF₂ |
| Me | Cl | H | Me | CH₂CF₃ |
| Me | Cl | H | Me | CH₂CF₃ |
| Me | Cl | H | Me | CH₂Cl |
| Me | Cl | H | Me | CClF₂ |
| Me | Cl | H | Me | CH₂CH₂Cl |
| Me | Cl | H | Me | n-C₃F₇ |
| Me | Cl | H | Me | i-C₃F₇ |
| Me | Cl | H | Me | Allyl |
| Me | Cl | H | Me | CF₂CHF₂ |
| Me | Cl | H | CF₃ | i-C₃F₇ |
| Me | Cl | H | CF₃ | CF₂CHF₂ |
| Me | Cl | H | OMe | CF₂CHF₂ |
| Me | Cl | H | H | CF₂CHF₂ |
| Me | Cl | H | H | Me |
| Me | Cl | H | H | CH₂CF₃ |
| Et | Cl | H | H | CH₂CF₃ |
| Me | Cl | H | H | CH₂CF₃ |
| Me | Cl | H | H | C₂F₅ |
| Me | Cl | H | H | C₂F₅ |
| Me | Cl | H | H | C₂F₅ |
| Me | Cl | H | H | CF₂CHF₂ |
| Me | Cl | H | H | CH₂CF₃ |
| Me | Cl | H | H | n-C₃F₇ |
| Me | Cl | H | 3-Me-2- | CH₂CF₃ |
| | | | pyridyl | |
| Me | Cl | H | H | i-C₃F₇ |
| Me | Cl | H | Ph | CH₂CF₃ |
| Me | Cl | H | Ph | CF₂CHF₂ |
| Me | Cl | H | Ph | CHF₂ |
| Me | Cl | H | 2-pyridyl | CH₂CF₃ |
| Me | Cl | H | 2-pyridyl | CF₂CHF₂ |
| Me | Cl | H | 2-ClPh | CH₂CF₃ |
| Me | Cl | H | 2-ClPh | CF₂CHF₂ |
| Me | Cl | H | 2-FPh | CH₂CF₃ |
| Me | Cl | H | 2,6-F₂Ph | CH₂CF₃ |
| Me | Cl | H | 2,4-F₂Ph | CH₂CF₃ |

TABLE 6

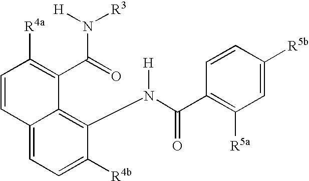

| R³ | R^{4a} | R^{4b} | R^{5a} | R^{5b} | R³ | R^{4a} | R^{4b} | R^{5a} | R^{5b} |
|---|---|---|---|---|---|---|---|---|---|
| Me | H | Me | H | CF₃ | Me | H | H | H | CF₃ |
| Me | H | Me | H | OCF₃ | Me | H | H | H | OCF₃ |
| Et | H | Me | H | OCF₃ | Et | H | H | H | OCF₃ |
| Me | H | Me | Me | Br | Me | H | H | Me | Br |
| Me | H | Me | Et | Br | Me | H | H | Et | Br |
| Me | H | Me | Me | Cl | Me | H | H | Me | Cl |
| Me | H | Me | Et | Cl | Me | H | H | Et | Cl |
| Me | H | Me | Me | I | Me | H | H | Me | I |
| Me | H | Me | Me | CF₃ | Me | H | H | Me | CF₃ |
| Me | H | Me | Me | OCF₃ | Me | H | H | Me | OCF₃ |
| Et | H | Me | Me | CF₃ | Et | H | H | Me | CF₃ |
| Me | H | Me | Me | SCF₃ | Me | H | H | Me | SCF₃ |
| Me | H | Me | Me | SCHF₂ | Me | H | H | Me | SCHF₂ |
| Me | H | Me | Me | OCHF₂ | Me | H | H | Me | OCHF₂ |
| n-Pr | H | Me | Me | CF₃ | n-Pr | H | H | Me | CF₃ |
| Me | H | Me | Me | C₂F₅ | Me | H | H | Me | C₂F₅ |
| Et | H | Me | Me | C₂F₅ | Et | H | H | Me | C₂F₅ |
| Me | H | Me | Et | CF₃ | Me | H | H | Et | CF₃ |
| Me | H | Me | n-Pr | CF₃ | Me | H | H | n-Pr | CF₃ |
| Me | H | Me | i-Pr | CF₃ | Me | H | H | i-Pr | CF₃ |
| Me | H | Me | Cl | CF₃ | Me | H | H | Cl | CF₃ |
| Me | H | Me | F | CF₃ | Me | H | H | F | CF₃ |
| Me | H | Me | Me | SMe | Me | H | H | Me | SMe |
| Me | H | Me | Me | OMe | Me | H | H | Me | OMe |
| Me | H | Me | Me | OEt | Me | H | H | Me | OEt |
| Me | H | Me | Me | n-C₃F₇ | Me | H | H | Me | n-C₃F₇ |
| Me | H | Me | Me | i-C₃F₇ | Me | H | H | Me | i-C₃F₇ |
| Me | H | Me | Me | Et | Me | H | H | Me | Et |
| Me | H | Me | Me | OCF₂CHF₂ | Me | H | H | Me | OCF₂CHF₂ |
| Me | H | Me | Me | SCF₂CHF₂ | Me | H | H | Me | SCF₂CHF₂ |
| Me | H | Me | Me | SO₂Me | Me | H | H | Me | SO₂Me |
| Me | H | Me | Me | SO₂CF₃ | Me | H | H | Me | SO₂CF₃ |
| Me | H | Me | CF₃ | CF₃ | Me | H | H | CF₃ | CF₃ |
| Me | H | Me | CF₃ | Me | Me | H | H | CF₃ | Me |
| Me | H | Me | OMe | CF₃ | Me | H | H | OMe | CF₃ |
| Me | H | Me | H | CF₃ | Me | H | H | H | CF₃ |
| Me | H | Me | H | OCHF₂ | Me | H | H | H | OCHF₂ |
| Me | H | Me | H | C₂F₅ | Me | H | H | H | C₂F₅ |
| Et | H | Me | H | C₂F₅ | Et | H | H | H | C₂F₅ |
| Me | H | Me | H | OCF₃ | Me | H | H | H | OCF₃ |
| Me | H | Me | H | OCF₂CHF₂ | Me | H | H | H | OCF₂CHF₂ |
| Me | H | Me | H | SCF₂CHF₂ | Me | H | H | H | SCF₂CHF₂ |
| Me | H | Me | H | n-C₃F₇ | Me | H | H | H | n-C₃F₇ |
| Me | H | Me | H | i-C₃F₇ | Me | H | H | H | i-C₃F₇ |
| Me | H | Me | H | Br | Me | H | H | H | Br |
| Me | H | Me | H | Cl | Me | H | H | H | Cl |
| Me | H | Me | H | SCF₃ | Me | H | H | H | SCF₃ |
| Me | H | Me | Ph | CF₃ | Me | H | H | Ph | CF₃ |
| Me | H | Me | Ph | Cl | Me | H | H | Ph | Cl |
| Me | H | Me | Ph | Br | Me | H | H | Ph | Br |
| Me | H | Me | 2-pyridyl | CF₃ | Me | H | H | 2-pyridyl | CF₃ |
| Me | H | Me | 2-pyridyl | Cl | Me | H | H | 2-pyridyl | Cl |
| Me | H | Me | 2-ClPh | CF₃ | Me | H | H | 2-ClPh | CF₃ |
| Me | H | Me | 2-ClPh | OCF₃ | Me | H | H | 2-ClPh | OCF₃ |
| Me | H | Me | 2-ClPh | Br | Me | H | H | 2-ClPh | Br |
| Me | H | Me | 2-ClPh | Cl | Me | H | H | 2-ClPh | Cl |
| Me | H | Me | 2-ClPh | SCHF₂ | Me | H | H | 2-ClPh | SCHF₂ |
| Me | H | Me | 2-BrPh | CF₃ | Me | H | H | 2-BrPh | CF₃ |
| Me | H | Me | 2-MePh | CF₃ | Me | H | H | 2-MePh | CF₃ |
| Me | H | Me | 2-CNPh | CF₃ | Me | H | H | 2-CNPh | CF₃ |
| Me | H | Me | 2-FPh | CF₃ | Me | H | H | 2-FPh | CF₃ |
| Me | H | Me | 2,6-F₂Ph | CF₃ | Me | H | H | 2,6-F₂Ph | CF₃ |
| Me | H | Me | 2,4-F₂Ph | CF₃ | Me | H | H | 2,4-F₂Ph | CF₃ |
| Me | H | Me | 2,5-F₂Ph | CF₃ | Me | H | H | 2,5-F₂Ph | CF₃ |
| Me | H | Me | 2-MeOPh | CF₃ | Me | H | H | 2-MeOPh | CF₃ |
| Me | H | Me | 3-Cl-2-pyridyl | CF₃ | Me | H | H | 3-Cl-2-pyridyl | CF₃ |
| Me | H | Me | 3-Cl-2-pyridyl | OCF₃ | Me | H | H | 3-Cl-2-pyridyl | OCF₃ |
| Me | H | Me | 3-Cl-2-pyridyl | Br | Me | H | H | 3-Cl-2-pyridyl | Br |
| Me | H | Me | 3-Cl-2-pyridyl | Cl | Me | H | H | 3-Cl-2-pyridyl | Cl |
| Me | H | Me | 3-Cl-2-pyridyl | SCHF₂ | Me | H | H | 3-Cl-2-pyridyl | SCHF₂ |
| Me | H | Me | 3-F-2-pyridyl | CF₃ | Me | H | H | 3-F-2-pyridyl | CF₃ |
| Me | H | Me | 3-CF₃-2- | CF₃ | Me | H | H | 3-CF₃-2- | CF₃ |
| | | | pyridyl | | | | | pyridyl | |
| Me | H | Me | 3-Me-2- | CF₃ | Me | H | H | 3-Me-2- | CF₃ |
| | | | pyridyl | | | | | pyridyl | |
| Me | H | Me | 3-Br-2-pyridyl | CF₃ | Me | H | H | 3-Br-2-pyridyl | CF₃ |
| Me | H | Me | 3-Br-2-pyridyl | OCF₃ | Me | H | H | 3-Br-2-pyridyl | OCF₃ |
| Me | H | Me | 3-Br-2-pyridyl | Br | Me | H | H | 3-Br-2-pyridyl | Br |
| Me | H | Me | 3-Br-2-pyridyl | Cl | Me | H | H | 3-Br-2-pyridyl | Cl |
| Me | Me | H | H | CF₃ | Me | H | Cl | Et | Br |
| Me | Me | H | H | OCF₃ | Me | H | Cl | Me | Cl |
| Et | Me | H | H | OCF₃ | Me | H | Cl | Et | Cl |
| Me | Me | H | Me | Br | Me | H | Cl | Me | I |
| Me | Me | H | Et | Br | Me | H | Cl | Me | CF₃ |
| Me | Me | H | Me | Cl | Me | H | Cl | Me | OCF₃ |
| Me | Me | H | Et | Cl | Et | H | Cl | Me | CF₃ |
| Me | Me | H | Me | I | Me | H | Cl | Me | SCF₃ |
| Me | Me | H | Me | CF₃ | Me | H | Cl | Me | SCHF₂ |
| Me | Me | H | Me | OCF₃ | Me | H | Cl | Me | OCHF₂ |
| Et | Me | H | Me | CF₃ | n-Pr | H | Cl | Me | CF₃ |
| Me | Me | H | Me | SCF₃ | Me | H | Cl | Me | C₂F₅ |
| Me | Me | H | Me | SCHF₂ | Et | H | Cl | Me | C₂F₅ |
| Me | Me | H | Me | OCHF₂ | Me | H | Cl | Et | CF₃ |
| n-Pr | Me | H | Me | CF₃ | Me | H | Cl | n-Pr | CF₃ |
| Me | Me | H | Me | C₂F₅ | Me | H | Cl | i-Pr | CF₃ |
| Et | Me | H | Me | C₂F₅ | Me | H | Cl | Cl | CF₃ |
| Me | Me | H | Et | CF₃ | Me | H | Cl | F | CF₃ |
| Me | Me | H | n-Pr | CF₃ | Me | H | Cl | Me | SMe |
| Me | Me | H | i-Pr | CF₃ | Me | H | Cl | Me | OMe |
| Me | Me | H | Cl | CF₃ | Me | H | Cl | Me | OEt |
| Me | Me | H | F | CF₃ | Me | H | Cl | Me | n-C₃F₇ |
| Me | Me | H | Me | SMe | Me | H | Cl | Me | i-C₃F₇ |
| Me | Me | H | Me | OMe | Me | H | Cl | Me | Et |
| Me | Me | H | Me | OEt | Me | H | Cl | Me | OCF₂CHF₂ |
| Me | Me | H | Me | n-C₃F₇ | Me | H | Cl | Me | SCF₂CHF₂ |
| Me | Me | H | Me | i-C₃F₇ | Me | H | Cl | Me | SO₂Me |
| Me | Me | H | Me | Et | Me | H | Cl | Me | SO₂CF₃ |
| Me | Me | H | Me | OCF₂CHF₂ | Me | H | Cl | CF₃ | CF₃ |
| Me | Me | H | Me | SCF₂CHF₂ | Me | H | Cl | CF₃ | Me |
| Me | Me | H | Me | SO₂Me | Me | H | Cl | OMe | CF₃ |
| Me | Me | H | Me | SO₂CF₃ | Me | H | Cl | H | CF₃ |
| Me | Me | H | CF₃ | CF₃ | Me | H | Cl | H | OCHF₂ |
| Me | Me | H | CF₃ | Me | Me | H | Cl | H | C₂F₅ |
| Me | Me | H | OMe | CF₃ | Et | H | Cl | H | C₂F₅ |
| Me | Me | H | H | CF₃ | Me | H | Cl | H | OCF₃ |
| Me | Me | H | H | OCHF₂ | Me | H | Cl | H | OCF₂CHF₂ |
| Me | Me | H | H | C₂F₅ | Me | H | Cl | H | SCF₂CHF₂ |
| Et | Me | H | H | C₂F₅ | Me | H | Cl | H | n-C₃F₇ |
| Me | Me | H | H | OCF₃ | Me | H | Cl | H | i-C₃F₇ |
| Me | Me | H | H | OCF₂CHF₂ | Me | H | Cl | H | Br |
| Me | Me | H | H | SCF₂CHF₂ | Me | H | Cl | H | Cl |
| Me | Me | H | H | n-C₃F₇ | Me | H | Cl | H | SCF₃ |
| Me | Me | H | H | i-C₃F₇ | Me | H | Cl | Ph | CF₃ |
| Me | Me | H | H | Br | Me | H | Cl | Ph | Cl |
| Me | Me | H | H | Cl | Me | H | Cl | Ph | Br |
| Me | Me | H | H | SCF₃ | Me | H | Cl | 2-pyridyl | CF₃ |
| Me | Me | H | Ph | CF₃ | Me | H | Cl | 2-pyridyl | Cl |
| Me | Me | H | Ph | Cl | Me | H | Cl | 2-ClPh | CF₃ |
| Me | Me | H | Ph | Br | Me | H | Cl | 2-ClPh | OCF₃ |
| Me | Me | H | 2-pyridyl | CF₃ | Me | H | Cl | 2-ClPh | Br |
| Me | Me | H | 2-pyridyl | Cl | Me | H | Cl | 2-ClPh | Cl |
| Me | Me | H | 2-ClPh | CF₃ | Me | H | Cl | 2-ClPh | SCHF₂ |
| Me | Me | H | 2-ClPh | OCF₃ | Me | H | Cl | 2-BrPh | CF₃ |
| Me | Me | H | 2-ClPh | Br | Me | H | Cl | 2-MePh | CF₃ |
| Me | Me | H | 2-ClPh | Cl | Me | H | Cl | 2-CNPh | CF₃ |
| Me | Me | H | 2-ClPh | SCHF₂ | Me | H | Cl | 2-FPh | CF₃ |
| Me | Me | H | 2-BrPh | CF₃ | Me | H | Cl | 2,6-F₂Ph | CF₃ |
| Me | Me | H | 2-MePh | CF₃ | Me | H | Cl | 2,4-F₂Ph | CF₃ |
| Me | Me | H | 2-CNPh | CF₃ | Me | H | Cl | 2.5-F₂Ph | CF₃ |
| Me | Me | H | 2-FPh | CF₃ | Me | H | Cl | 2-MeOPh | CF₃ |
| Me | Me | H | 2,6-F₂Ph | CF₃ | Me | H | Cl | 3-Cl-2-pyridyl | CF₃ |
| Me | Me | H | 2,4-F₂Ph | CF₃ | Me | H | Cl | 3-Cl-2-pyridyl | OCF₃ |
| Me | Me | H | 2,5-F₂Ph | CF₃ | Me | H | Cl | 3-Cl-2-pyridyL | Br |
| Me | Me | H | 2-MeOPh | CF₃ | Me | H | Cl | 3-Cl-2-pyridyl | Cl |
| Me | Me | H | 3-Cl-2-pyridyl | CF₃ | Me | H | Cl | 3-Cl-2-pyridyl | SCHF₂ |
| Me | Me | H | 3-Cl-2-pyridyl | OCF₃ | Me | H | Cl | 3-F-2-pyridyl | CF₃ |
| Me | Me | H | 3-Cl-2-pyridyl | Br | Me | H | Cl | 3-Br-2-pyridyl | CF₃ |
| Me | Me | H | 3-Cl-2-pyridyl | Cl | Me | H | Cl | 3-Br-2-pyridyl | OCF₃ |
| Me | Me | H | 3-Cl-2-pyridyl | SCHF₂ | Me | H | Cl | 3-Br-2-pyridyl | Br |
| Me | Me | H | 3-F-2-pyridyl | CF₃ | Me | H | Cl | 3-Br-2-pyridyl | Cl |
| Me | Me | H | 3-CF₃-2- | CF₃ | Me | H | Cl | 3-CF₃-2- | CF₃ |
| | | | pyridyl | | | | | pyridyl | |
| Me | Me | H | 3-Me-2- | CF₃ | Me | H | Cl | 3-Me-2- | CF₃ |
| | | | pyridyl | | | | | pyridyl | |
| Me | Me | H | 3-Br-2-pyridyl | CF₃ | Me | Cl | H | H | OCF₃ |
| Me | Me | H | 3-Br-2-pyridyl | OCF₃ | Me | Cl | H | H | OCF₂CHF₂ |
| Me | Me | H | 3-Br-2-pyridyl | Br | Me | Cl | H | H | SCF₂CHF₂ |
| Me | Me | H | 3-Br-2-pyridyl | Cl | Me | Cl | H | H | n-C₃F₇ |
| Me | Cl | H | Me | Br | Me | Cl | H | H | i-C₃F₇ |
| Me | Cl | H | Et | Br | Me | Cl | H | H | Br |
| Me | Cl | H | Me | Cl | Me | Cl | H | H | Cl |
| Me | Cl | H | Et | Cl | Me | Cl | H | H | SCF₃ |
| Me | Cl | H | Me | I | Me | Cl | H | Ph | CF₃ |
| Me | Cl | H | Me | CF₃ | Me | Cl | H | Ph | Cl |
| Me | Cl | H | Me | OCF₃ | Me | Cl | H | Ph | Br |
| Et | Cl | H | Me | CF₃ | Me | Cl | H | 2-pyridyl | CF₃ |
| Me | Cl | H | Me | SCF₃ | Me | Cl | H | 2-pyridyl | Cl |
| Me | Cl | H | Me | SCHF₂ | Me | Cl | H | 2-ClPh | CF₃ |
| Me | Cl | H | Me | OCHF₂ | Me | Cl | H | 2-ClPh | OCF₃ |
| n-Pr | Cl | H | Me | CF₃ | Me | Cl | H | 2-ClPh | Br |
| Me | Cl | H | Me | C₂F₅ | Me | Cl | H | 2-ClPh | Cl |
| Et | Cl | H | Me | C₂F₅ | Me | Cl | H | 2-ClPh | SCHF₂ |
| Me | Cl | H | Et | CF₃ | Me | Cl | H | 2-BrPh | CF₃ |
| Me | Cl | H | n-Pr | CF₃ | Me | Cl | H | 2-MePh | CF₃ |
| Me | Cl | H | i-Pr | CF₃ | Me | Cl | H | 2-CNPh | CF₃ |
| Me | Cl | H | Cl | CF₃ | Me | Cl | H | 2-FPh | CF₃ |
| Me | Cl | H | F | CF₃ | Me | Cl | H | 2,6-F₂Ph | CF₃ |
| Me | Cl | H | Me | SMe | Me | Cl | H | 2,4-F₂Ph | CF₃ |
| Me | Cl | H | Me | OMe | Me | Cl | H | 2,5-F₂Ph | CF₃ |
| Me | Cl | H | Me | OEt | Me | Cl | H | 2-MeOPh | CF₃ |
| Me | Cl | H | Me | n-G₃F₇ | Me | Cl | H | 3-Cl-2-pyridyl | CF₃ |
| Me | Cl | H | Me | i-C₃F₇ | Me | Cl | H | 3-Cl-2-pyridyl | OCF₃ |
| Me | Cl | H | Me | Et | Me | Cl | H | 3-Cl-2-pyridyl | Br |
| Me | Cl | H | Me | OCF₂CHF₂ | Me | Cl | H | 3-Cl-2-pyridyl | Cl |
| Me | Cl | H | Me | SCF₂CHF₂ | Me | Cl | H | 3-Cl-2-pyridyl | SCHF₂ |
| Me | Cl | H | Me | SO₂Me | Me | Cl | H | 3-F-2-pyridyl | CF₃ |
| Me | Cl | H | Me | SO₂CF₃ | Me | Cl | H | CF₃ | CF₃ |
| Me | Cl | H | 3-CF₃-2- | CF₃ | Me | Cl | H | 3-Me-2- | CF₃ |
| | | | pyridyl | | | | | pyridyl | |
| Me | Cl | H | CF₃ | Me | Me | Cl | H | 3-Br-2-pyridyl | CF₃ |
| Me | Cl | H | OMe | CF₃ | Me | Cl | H | 3-Br-2-pyridyl | OCF₃ |
| Me | Cl | H | H | CF₃ | Me | Cl | H | 3-Br-2-pyridyl | Br |
| Me | Cl | H | H | OCHF₂ | Me | Cl | H | 3-Br-2-pyridyl | Cl |
| Me | Cl | H | H | C₂F₅ | Et | Cl | H | H | C₂F₅ |

TABLE 7

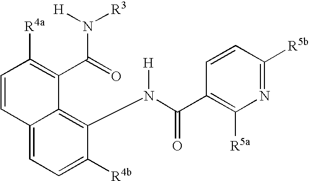

| R³ | R^{4a} | R^{4b} | R^{5a} | R^{5b} | R³ | R^{4a} | R^{4b} | R^{5a} | R^{5b} |
|---|---|---|---|---|---|---|---|---|---|
| Me | H | Me | H | CF₃ | Me | H | H | H | CF₃ |
| Me | H | Me | H | OCF₃ | Me | H | H | H | OCF₃ |
| Et | H | Me | H | OCF₃ | Et | H | H | H | OCF₃ |
| Me | H | Me | Me | Br | Me | H | H | Me | Br |
| Me | H | Me | Et | Br | Me | H | H | Et | Br |
| Me | H | Me | Me | CI | Me | H | H | Me | Cl |
| Me | H | Me | Et | Cl | Me | H | H | Et | Cl |
| Me | H | Me | Me | I | Me | H | H | Me | I |
| Me | H | Me | Me | CF₃ | Me | H | H | Me | CF₃ |
| Me | H | Me | Me | OCF₃ | Me | H | H | Me | OCF₃ |
| Et | H | Me | Me | CF₃ | Et | H | H | Me | CF₃ |
| Me | H | Me | Me | SCF₃ | Me | H | H | Me | SCF₃ |
| Me | H | Me | Me | SCHF₂ | Me | H | H | Me | SCHF₂ |
| Me | H | Me | Me | OCHF₂ | Me | H | H | Me | OCHF₂ |
| n-Pr | H | Me | Me | CF₃ | n-Pr | H | H | Me | CF₃ |
| Me | H | Me | Me | C₂F₅ | Me | H | H | Me | C₂F₅ |
| Et | H | Me | Me | C₂F₅ | Et | H | H | Me | C₂F₅ |
| Me | H | Me | Et | CF₃ | Me | H | H | Et | CF₃ |
| Me | H | Me | n-Pr | CF₃ | Me | H | H | n-Pr | CF₃ |
| Me | H | Me | i-Pr | CF₃ | Me | H | H | i-Pr | CF₃ |
| Me | H | Me | Cl | CF₃ | Me | H | H | Cl | CF₃ |
| Me | H | Me | F | CF₃ | Me | H | H | F | CF₃ |
| Me | H | Me | Me | SMe | Me | H | H | Me | SMe |
| Me | H | Me | Me | OMe | Me | H | H | Me | OMe |
| Me | H | Me | Me | OEt | Me | H | H | Me | OEt |
| Me | H | Me | Me | n-C₃F₇ | Me | H | H | Me | n-C₃F₇ |
| Me | H | Me | Me | i-C₃F₇ | Me | H | H | Me | i-C₃F₇ |
| Me | H | Me | Me | Et | Me | H | H | Me | Et |
| Me | H | Me | Me | OCF₂CHF₂ | Me | H | H | Me | OCF₂CHF₂ |
| Me | H | Me | Me | SCF₂CHF₂ | Me | H | H | Me | SCF₂CHF₂ |
| Me | H | Me | Me | SO₂Me | Me | H | H | Me | SO₂Me |
| Me | H | Me | Me | SO₂CF₃ | Me | H | H | Me | SO₂CF₃ |
| Me | H | Me | CF₃ | CF₃ | Me | H | H | CF₃ | CF₃ |
| Me | H | Me | CF₃ | Me | Me | H | H | CF₃ | Me |
| Me | H | Me | OMe | CF₃ | Me | H | H | OMe | CF₃ |
| Me | H | Me | H | CF₃ | Me | H | H | H | CF₃ |
| Me | H | Me | H | OCHF₂ | Me | H | H | H | OCHF₂ |
| Me | H | Me | H | C₂F₅ | Me | H | H | H | C₂F₅ |
| Et | H | Me | H | C₂F₅ | Et | H | H | H | C₂F₅ |
| Me | H | Me | H | OCF₃ | Me | H | H | H | OCF₃ |
| Me | H | Me | H | OCF₂CHF₂ | Me | H | H | H | OCF₂CHF₂ |
| Me | H | Me | H | SCF₂CHF₂ | Me | H | H | H | SCF₂CHF₂ |
| Me | H | Me | H | n-C₃F₇ | Me | H | H | H | n-C₃F₇ |
| Me | H | Me | H | i-C₃F₇ | Me | H | H | H | i-C₃F₇ |
| Me | H | Me | H | Br | Me | H | H | H | Br |
| Me | H | Me | H | Cl | Me | H | H | H | Cl |
| Me | H | Me | H | SCF₃ | Me | H | H | H | SCF₃ |
| Me | H | Me | Ph | CF₃ | Me | H | H | Ph | CF₃ |
| Me | H | Me | Ph | Cl | Me | H | H | Ph | Cl |
| Me | H | Me | Ph | Br | Me | H | H | Ph | Br |
| Me | H | Me | 2-pyridyl | CF₃ | Me | H | H | 2-pyridyl | CF₃ |
| Me | H | Me | 2-pyridyl | Cl | Me | H | H | 2-pyridyl | Cl |
| Me | H | Me | 2-ClPh | CF₃ | Me | H | H | 2-ClPh | CF₃ |
| Me | H | Me | 2-ClPh | OCF₃ | Me | H | H | 2-ClPh | OCF₃ |
| Me | H | Me | 2-Clph | Br | Me | H | H | 2-ClPh | Br |
| Me | H | Me | 2-ClPh | Cl | Me | H | H | 2-ClPh | Cl |
| Me | H | Me | 2-ClPh | SCHF₂ | Me | H | H | 2-ClPh | SCHF₂ |
| Me | H | Me | 2-BrPh | CF₃ | Me | H | H | 2-BrPh | CF₃ |
| Me | H | Me | 2-MePh | CF₃ | Me | H | H | 2-MePh | CF₃ |
| Me | H | Me | 2-CNPh | CF₃ | Me | H | H | 2-CNPh | CF₃ |
| Me | H | Me | 2-FPh | CF₃ | Me | H | H | 2-FPh | CF₃ |
| Me | H | Me | 2,6-F₂Ph | CF₃ | Me | H | H | 2,6-F₂Ph | CF₃ |
| Me | H | Me | 2,4-F₂Ph | CF₃ | Me | H | H | 2,4-F₂Ph | CF₃ |
| Me | H | Me | 2,5-F₂Ph | CF₃ | Me | H | H | 2,5-F₂Ph | CF₃ |
| Me | H | Me | 2-MeOPh | CF₃ | Me | H | H | 2-MeOPh | CF₃ |
| Me | H | Me | 3-Cl-2-pyridyl | CF₃ | Me | H | H | 3-Cl-2-pyridyl | CF₃ |
| Me | H | Me | 3-Cl-2-pyridyl | OCF₃ | Me | H | H | 3-Cl-2-pyridyl | OCF₃ |
| Me | H | Me | 3-Cl-2-pyridyl | Br | Me | H | H | 3-Cl-2-pyridyl | Br |
| Me | H | Me | 3-Cl-2-pyridyl | Cl | Me | H | H | 3-Cl-2-pyridyl | Cl |
| Me | H | Me | 3-Cl-2-pyridyl | SCHF₂ | Me | H | H | 3-Cl-2-pyridyl | SCHF₂ |
| Me | H | Me | 3-F-2-pyridyl | CF₃ | Me | H | H | 3-F-2-pyridyl | CF₃ |
| Me | H | Me | 3-CF₃-2- | CF₃ | Me | H | H | 3-CF₃-2- | CF₃ |
| | | | pyridyl | | | | | pyridyl | |
| Me | H | Me | 3-Me-2- | CF₃ | Me | H | H | 3-Me-2- | CF₃ |
| | | | pyridyl | | | | | pyridyl | |
| Me | H | Me | 3-Br-2-pyridyl | CF₃ | Me | H | H | 3-Br-2-pyridyl | CF₃ |
| Me | H | Me | 3-Br-2-pyridyl | OCF₃ | Me | H | H | 3-Br-2-pyridyl | OCF₃ |
| Me | H | Me | 3-Br-2-pyridyl | Br | Me | H | H | 3-Br-2-pyridyl | Br |
| Me | H | Me | 3-Br-2-pyridyl | Cl | Me | H | H | 3-Br-2-pyridyl | Cl |
| Me | Me | H | H | CF₃ | Me | H | Cl | Et | Br |
| Me | Me | H | H | OCF₃ | Me | H | Cl | Me | Cl |
| Et | Me | H | H | OCF₃ | Me | H | Cl | Et | Cl |
| Me | Me | H | Me | Br | Me | H | Cl | Me | I |
| Me | Me | H | El | Br | Me | H | Cl | Me | CF₃ |
| Me | Me | H | Me | Cl | Me | H | Cl | Me | OCF₃ |
| Me | Me | H | Et | Cl | Et | H | Cl | Me | CF₃ |
| Me | Me | H | Me | I | Me | H | Cl | Me | SCF₃ |
| Me | Me | H | Me | CF₃ | Me | H | Cl | Me | SCHF₂ |
| Me | Me | H | Me | OCF₃ | Me | H | Cl | Me | OCHF₂ |
| Et | Me | H | Me | CF₃ | n-Pr | H | Cl | Me | CF₃ |
| Me | Me | H | Me | SCF₃ | Me | H | Cl | Me | C₂F₅ |
| Me | Me | H | Me | SCHF₂ | Et | H | Cl | Me | C₂F₅ |
| Me | Me | H | Me | OCHF₂ | Me | H | Cl | Et | CF₃ |
| n-Pr | Me | H | Me | CF₃ | Me | H | Cl | n-Pr | CF₃ |
| Me | Me | H | Me | C₂F₅ | Me | H | Cl | i-Pr | CF₃ |
| Et | Me | H | Me | C₂F₅ | Me | H | Cl | Cl | CF₃ |
| Me | Me | H | Et | CF₃ | Me | H | Cl | F | CF₃ |
| Me | Me | H | n-Pr | CF₃ | Me | H | Cl | Me | SMe |
| Me | Me | H | i-Pr | CF₃ | Me | H | Cl | Me | OMe |
| Me | Me | H | Cl | CF₃ | Me | H | Cl | Me | OEt |
| Me | Me | H | F | CF₃ | Me | H | Cl | Me | n-C₃F₇ |
| Me | Me | H | Me | SMe | Me | H | Cl | Me | i-C₃F₇ |
| Me | Me | H | Me | OMe | Me | H | Cl | Me | Et |
| Me | Me | H | Me | OEt | Me | H | Cl | Me | OCF₂CHF₂ |
| Me | Me | H | Me | n-C₃F₇ | Me | H | Cl | Me | SCF₂CHF₂ |
| Me | Me | H | Me | i-C₃F₇ | Me | H | Cl | Me | SO₂Me |
| Me | Me | H | Me | Et | Me | H | Cl | Me | SO₂CF₃ |
| Me | Me | H | Me | OCF₂CHF₂ | Me | H | Cl | CF₃ | CF₃ |
| Me | Me | H | Me | SCF₂CHF₂ | Me | H | Cl | CF₃ | Me |
| Me | Me | H | Me | SO₂Me | Me | H | Cl | OMe | CF₃ |
| Me | Me | H | Me | SO₂CF₃ | Me | H | Cl | H | CF₃ |
| Me | Me | H | CF₃ | CF₃ | Me | H | Cl | H | OCHF₂ |
| Me | Me | H | CF₃ | Me | Me | H | Cl | H | C₂F₅ |
| Me | Me | H | OMe | CF₃ | Et | H | Cl | H | C₂F₅ |
| Me | Me | H | H | CF₃ | Me | H | Cl | H | OCF₃ |
| Me | Me | H | H | OCHF₂ | Me | H | Cl | H | OCF₂CHF₂ |
| Me | Me | H | H | C₂F₅ | Me | H | Cl | H | SCF₂CHF₂ |
| Et | Me | H | H | C₂F₅ | Me | H | Cl | H | n-C₃F₇ |
| Me | Me | H | H | OCF₃ | Me | H | Cl | H | i-C₃F₇ |
| Me | Me | H | H | OCF₂CHF₂ | Me | H | Cl | H | Br |
| Me | Me | H | H | SCF₂CHF₂ | Me | H | Cl | H | Cl |
| Me | Me | H | H | n-C₃F₇ | Me | H | Cl | H | SCF₃ |
| Me | Me | H | H | i-C₃F₇ | Me | H | Cl | Ph | CF₃ |
| Me | Me | H | H | Br | Me | H | Cl | Ph | Cl |
| Me | Me | H | H | Cl | Me | H | Cl | Ph | Br |
| Me | Me | H | H | SCF₃ | Me | H | Cl | 2-pyridyl | CF₃ |
| Me | Me | H | Ph | CF₃ | Me | H | Cl | 2-pyridyl | Cl |
| Me | Me | H | Ph | Cl | Me | H | Cl | 2-ClPh | CF₃ |
| Me | Me | H | Ph | Br | Me | H | Cl | 2-ClPh | OCF₃ |
| Me | Me | H | 2-pyridyl | CF₃ | Me | H | Cl | 2-ClPh | Br |
| Me | Me | H | 2-pyridyl | Cl | Me | H | Cl | 2-ClPh | Cl |
| Me | Me | H | 2-ClPh | CF₃ | Me | H | Cl | 2-ClPh | SCHF₂ |
| Me | Me | H | 2-ClPh | OCF₃ | Me | H | Cl | 2-BrPh | CF₃ |
| Me | Me | H | 2-ClPh | Br | Me | H | Cl | 2-MePh | CF₃ |
| Me | Me | H | 2-ClPh | Cl | Me | Et | Cl | 2-CNPh | CF₃ |
| Me | Me | H | 2-ClPh | SCHF₂ | Me | H | Cl | 2-FPh | CF₃ |
| Me | Me | H | 2-BrPh | CF₃ | Me | H | Cl | 2,6-F₂Ph | CF₃ |
| Me | Me | H | 2-MePh | CF₃ | Me | H | Cl | 2,4-F₂Ph | CF₃ |
| Me | Me | H | 2-CNPh | CF₃ | Me | H | Cl | 2,5-F₂Ph | CF₃ |
| Me | Me | H | 2-FPh | CF₃ | Me | H | Cl | 2-MeOPh | CF₃ |
| Me | Me | H | 2,6-F₂Ph | CF₃ | Me | H | Cl | 3-Cl-2-pyridyl | CF₃ |
| Me | Me | H | 2,4-F₂Ph | CF₃ | Me | H | Cl | 3-Cl-2-pyridyl | OCF₃ |
| Me | Me | H | 2,5-F₂Ph | CF₃ | Me | H | Cl | 3-Cl-2-pyridyl | Br |
| Me | Me | H | 2-MeOPh | CF₃ | Me | H | Cl | 3-Cl-2-pyridyl | Cl |
| Me | Me | H | 3-Cl-2-pyridyl | CF₃ | Me | H | Cl | 3-Cl-2-pyridyl | SCHF₂ |
| Me | Me | H | 3-Cl-2-pyridyl | OCF₃ | Me | H | Cl | 3-F-2-pyridyl | CF₃ |
| Me | Me | H | 3-Cl-2-pyridyl | Br | Me | Me | H | 3-Cl-2-pyridyl | Cl |
| Me | H | Cl | 3-CF₃-2- | CF₃ | Me | H | Cl | 3-Me-2- | CF₃ |
| | | | pyridyl | | | | | pyridyl | |
| Me | Me | H | 3-Cl-2-pyridyl | SCHF₂ | Me | H | Cl | 3-Br-2-pyridyl | CF₃ |
| Me | Me | H | 3-F-2-pyridyl | CF₃ | Me | H | Cl | 3-Br-2-pyridyl | OCF₃ |
| Me | Me | H | 3-CF₃-2- | CF₃ | Me | Me | H | 3-Me-2- | CF₃ |
| | | | pyridyl | | | | | pyridyl | |
| Me | H | Cl | 3-Br-2-pyridyl | Br | Me | H | Cl | 3-Br-2-pyridyl | Cl |
| Me | Me | H | 3-Br-2-pyridyl | CF₃ | Me | Cl | H | H | OCF₃ |
| Me | Me | H | 3-Br-2-pyridyl | OCF₃ | Me | Cl | H | H | OCF₂CHF₂ |
| Me | Me | H | 3-Br-2-pyridyl | Br | Me | Cl | H | H | SCF₂CHF₂ |
| Me | Me | H | 3-Br-2-pyridyl | Cl | Me | Cl | H | H | n-C₃F₇ |
| Me | Cl | H | Me | Br | Me | Cl | H | H | i-C₃F₇ |
| Me | Cl | H | Et | Br | Me | Cl | H | H | Br |
| Me | Cl | H | Me | Cl | Me | Cl | H | H | Cl |
| Me | Cl | H | Et | Cl | Me | Cl | H | H | SCF₃ |
| Me | Cl | H | Me | I | Me | Cl | H | Ph | CF₃ |
| Me | Cl | H | Me | CF₃ | Me | Cl | H | Ph | Cl |
| Me | Cl | H | Me | OCF₃ | Me | Cl | H | Ph | Br |
| Et | Cl | H | Me | CF₃ | Me | Cl | H | 2-pyridyl | CF₃ |
| Me | Cl | H | Me | SCF₃ | Me | Cl | H | 2-pyridyl | Cl |
| Me | Cl | H | Me | SCHF₂ | Me | Cl | H | 2-ClPh | CF₃ |
| Me | Cl | H | Me | OCHF₂ | Me | Cl | H | 2-ClPh | OCF₃ |
| n-Pr | Cl | H | Me | CF₃ | Me | Cl | H | 2-ClPh | Br |
| Me | Cl | H | Me | C₂F₅ | Me | Cl | H | 2-ClPh | Cl |
| Et | Cl | H | Me | C₂F₅ | Me | Cl | H | 2-ClPh | SCHF₂ |
| Me | Cl | H | Et | CF₃ | Me | Cl | H | 2-BrPh | CF₃ |
| Me | Cl | H | n-Pr | CF₃ | Me | Cl | H | 2-MePh | CF₃ |
| Me | Cl | H | i-Pr | CF₃ | Me | Cl | H | 2-CNPh | CF₃ |
| Me | Cl | H | Cl | CF₃ | Me | Cl | H | 2-FPh | CF₃ |
| Me | Cl | H | F | CF₃ | Me | Cl | H | 2,6-F₂Ph | CF₃ |
| Me | Cl | H | Me | SMe | Me | Cl | H | 2,4-F₂Ph | CF₃ |
| Me | Cl | H | Me | OMe | Me | Cl | H | 2,5-F₂Ph | CF₃ |
| Me | Cl | H | Me | OEt | Me | Cl | H | 2-MeOPh | CF₃ |
| Me | Cl | H | Me | n-C₃F₇ | Me | Cl | H | 3-Cl-2-pyridyl | CF₃ |
| Me | Cl | H | Me | i-C₃F₇ | Me | Cl | H | 3-Cl-2-pyridyl | OCF₃ |
| Me | Cl | H | Me | Et | Me | Cl | H | 3-Cl-2-pyridyl | Br |
| Me | Cl | H | Me | OCF₂CHF₂ | Me | Cl | H | 3-Cl-2-pyridyl | Cl |
| Me | Cl | H | Me | SCF₂CHF₂ | Me | Cl | H | 3-Cl-2-pyridyl | SCHF₂ |
| Me | Cl | H | Me | SO₂Me | Me | Cl | H | 3-F-2-pyridyl | CF₃ |
| Me | Cl | H | 3-Me-2- | CF₃ | Me | Cl | H | 3-CF₃-2- | CF₃ |
| | | | pyridyl | | | | | pyridyl | |
| Me | Cl | H | CF₃ | CF₃ | Me | Cl | H | Me | SO₂CF₃ |
| Me | Cl | H | CF₃ | Me | Me | Cl | H | 3-Br-2-pyridyl | CF₃ |
| Me | Cl | H | OMe | CF₃ | Me | Cl | H | 3-Br-2-pyridyl | OCF₃ |
| Me | Cl | H | H | CF₃ | Me | Cl | H | 3-Br-2-pyridyl | Br |
| Me | Cl | H | H | OCHF₂ | Me | Cl | H | 3-Br-2-pyridyl | Cl |
| Me | Cl | H | H | C₂F₅ | Et | Cl | H | H | C₂F₅ |

TABLE 8

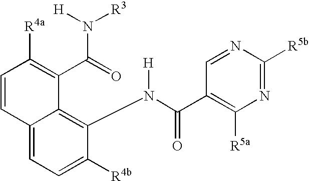

| R³ | R^{4a} | R^{4b} | R^{5a} | R^{5b} | R³ | R^{4a} | R^{4b} | R^{5a} | R^{5b} |
|---|---|---|---|---|---|---|---|---|---|
| Me | H | Me | H | CF₃ | Me | H | H | H | CF₃ |
| Me | H | Me | H | OCF₃ | Me | H | H | H | OCF₃ |
| Et | H | Me | H | OCF₃ | Et | H | H | H | OCF₃ |
| Me | H | Me | Me | Br | Me | H | H | Me | Br |
| Me | H | Me | Et | Br | Me | H | H | Et | Br |
| Me | H | Me | Me | Cl | Me | H | H | Me | Cl |
| Me | H | Me | Et | Cl | Me | H | H | Et | Cl |
| Me | H | Me | Me | I | Me | H | H | Me | I |
| Me | H | Me | Me | CF₃ | Me | H | H | Me | CF₃ |
| Me | H | Me | Me | OCF₃ | Me | H | H | Me | OCF₃ |
| Et | H | Me | Me | CF₃ | Et | H | H | Me | CF₃ |
| Me | H | Me | Me | SCF₃ | Me | H | H | Me | SCF₃ |
| Me | H | Me | Me | SCHF₂ | Me | H | H | Me | SCHF₂ |
| Me | H | Me | Me | OCHF₂ | Me | H | H | Me | OCHF₂ |
| n-Pr | H | Me | Me | CF₃ | n-Pr | H | H | Me | CF₃ |
| Me | H | Me | Me | C₂F₅ | Me | H | H | Me | C₂F₅ |
| Et | H | Me | Me | C₂F₅ | Et | H | H | Me | C₂F₅ |
| Me | H | Me | Et | CF₃ | Me | H | H | Et | CF₃ |
| Me | H | Me | n-Pr | CF₃ | Me | H | H | n-Pr | CF₃ |
| Me | H | Me | i-Pr | CF₃ | Me | H | H | i-Pr | CF₃ |
| Me | H | Me | Cl | CF₃ | Me | H | H | Cl | CF₃ |
| Me | H | Me | F | CF₃ | Me | H | H | F | CF₃ |
| Me | H | Me | Me | SMe | Me | H | H | Me | SMe |
| Me | H | Me | Me | OMe | Me | H | H | Me | OMe |
| Me | H | Me | Me | OEt | Me | H | H | Me | OEt |
| Me | H | Me | Me | n-C₃F₇ | Me | H | H | Me | n-C₃F₇ |
| Me | H | Me | Me | i-C₃F₇ | Me | H | H | Me | i-C₃F₇ |
| Me | H | Me | Me | Et | Me | H | H | Me | Et |
| Me | H | Me | Me | OCF₂CHF₂ | Me | H | H | Me | OCF₂CHF₂ |
| Me | H | Me | Me | SCF₂CHF₂ | Me | H | H | Me | SCF₂CHF₂ |
| Me | H | Me | Me | SO₂Me | Me | H | H | Me | SO₂Me |
| Me | H | Me | Me | SO₂CF₃ | Me | H | H | Me | SO₂CF₃ |
| Me | H | Me | CF₃ | CF₃ | Me | H | H | CF₃ | CF₃ |
| Me | H | Me | CF₃ | Me | Me | H | H | CF₃ | Me |
| Me | H | Me | OMe | CF₃ | Me | H | H | OMe | CF₃ |
| Me | H | Me | H | CF₃ | Me | H | H | H | CF₃ |
| Me | H | Me | H | OCHF₂ | Me | H | H | H | OCHF₂ |
| Me | H | Me | H | C₂F₅ | Me | H | H | H | C₂F₅ |
| Et | H | Me | H | C₂F₅ | Et | H | H | H | C₂F₅ |
| Me | H | Me | H | OCF₃ | Me | H | H | H | OCF₃ |
| Me | H | Me | H | OCF₂CHF₂ | Me | H | H | H | OCF₂CHF₂ |
| Me | H | Me | H | SCF₂CHF₂ | Me | H | H | H | SCF₂CHF₂ |
| Me | H | Me | H | n-C₃F₇ | Me | H | H | H | n-C₃F₇ |
| Me | H | Me | H | i-C₃F₇ | Me | H | H | H | i-C₃F₇ |
| Me | H | Me | H | Br | Me | H | H | H | Br |
| Me | H | Me | H | Cl | Me | H | H | H | Cl |
| Me | H | Me | H | SCF₃ | Me | H | H | H | SCF₃ |
| Me | H | Me | Ph | CF₃ | Me | H | H | Ph | CF₃ |
| Me | H | Me | Ph | Cl | Me | H | H | Ph | Cl |
| Me | H | Me | Ph | Br | Me | H | H | Ph | Br |
| Me | H | Me | 2-pyridyl | CF₃ | Me | H | H | 2-pyridyl | CF₃ |
| Me | H | Me | 2-pyridyl | Cl | Me | H | H | 2-pyridyl | Cl |
| Me | H | Me | 2-ClPh | CF₃ | Me | H | H | 2-ClPh | CF₃ |
| Me | H | Me | 2-ClPh | OCF₃ | Me | H | H | 2-ClPh | OCF₃ |
| Me | H | Me | 2-ClPh | Br | Me | H | H | 2-ClPh | Br |
| Me | H | Me | 2-ClPh | Cl | Me | H | H | 2-ClPh | Cl |
| Me | H | Me | 2-ClPh | SCHF₂ | Me | H | H | 2-ClPh | SCHF₂ |
| Me | H | Me | 2-BrPh | CF₃ | Me | H | H | 2-BrPh | CF₃ |
| Me | H | Me | 2-MePh | CF₃ | Me | H | H | 2-MePh | CF₃ |
| Me | H | Me | 2-CNPh | CF₃ | Me | H | H | 2-CNPh | CF₃ |
| Me | H | Me | 2-FPh | CF₃ | Me | H | H | 2-FPh | CF₃ |
| Me | H | Me | 2,6-F₂Ph | CF₃ | Me | H | H | 2,6-F₂Ph | CF₃ |
| Me | H | Me | 2,4-F₂Ph | CF₃ | Me | H | H | 2,4-F₂Ph | CF₃ |
| Me | H | Me | 2,5-F₂Ph | CF₃ | Me | H | H | 2,5-F₂Ph | CF₃ |
| Me | H | Me | 2-MeOPh | CF₃ | Me | H | H | 2-MeOPh | CF₃ |
| Me | H | Me | 3-Cl-2-pyridyl | CF₃ | Me | H | H | 3-Cl-2-pyridyl | CF₃ |
| Me | H | Me | 3-Cl-2-pyriddyl | OCF₃ | Me | H | H | 3-Cl-2-pyridyl | OCF₃ |
| Me | H | Me | 3-Cl-2-pyridyl | Br | Me | H | H | 3-Cl-2-pyridyl | Br |
| Me | H | Me | 3-Cl-2-pyridyl | Cl | Me | H | H | 3-Cl-2-pyridyl | Cl |
| Me | H | Me | 3-Cl-2-pyridyl | SCHF₂ | Me | H | H | 3-Cl-2-pyridyl | SCHF₂ |
| Me | H | Me | 3-F-2-pyridyl | CF₃ | Me | H | H | 3-F-2-pyridyl | CF₃ |
| Me | H | Me | 3-CF₃-2- | CF₃ | Me | H | H | 3-CF₃-2- | CF₃ |
| | | | pyridyl | | | | | pyridyl | |
| Me | H | Me | 3-Me-2- | CF₃ | Me | H | H | 3-Me-2- | CF₃ |
| | | | pyridyl | | | | | pyridyl | |
| Me | H | Me | 3-Br-2-pyridyl | CF₃ | Me | H | H | 3-Br-2-pyridyl | CF₃ |
| Me | H | Me | 3-Br-2-pyridyl | OCF₃ | Me | H | H | 3-Br-2-pyridyl | OCF₃ |
| Me | H | Me | 3-Br-2-pyridyl | Br | Me | H | H | 3-Br-2-pyridyl | Br |
| Me | H | Me | 3-Br-2-pyridyl | Cl | Me | H | H | 3-Br-2-pyridyl | Cl |
| Me | Me | H | H | CF₃ | Me | H | Cl | Et | Br |
| Me | Me | H | H | OCF₃ | Me | H | Cl | Me | Cl |
| Et | Me | H | H | OCF₃ | Me | H | Cl | Et | Cl |
| Me | Me | H | Me | Br | Me | H | Cl | Me | I |
| Me | Me | H | Et | Br | Me | H | Cl | Me | CF₃ |
| Me | Me | H | Me | Cl | Me | H | Cl | Me | OCF₃ |
| Me | Me | H | Et | Cl | Et | H | Cl | Me | CF₃ |
| Me | Me | H | Me | I | Me | H | Cl | Me | SCF₃ |
| Me | Me | H | Me | CF₃ | Me | H | Cl | Me | SCHF₂ |
| Me | Me | H | Me | OCF₃ | Me | H | Cl | Me | OCHF₂ |
| Et | Me | H | Me | CF₃ | n-Pr | H | Cl | Me | CF₃ |
| Me | Me | H | Me | SCF₃ | Me | H | Cl | Me | C₂F₅ |
| Me | Me | H | Me | SCHF₂ | Et | H | Cl | Me | C₂F₅ |
| Me | Me | H | Me | OCHF₂ | Me | H | Cl | Et | CF₃ |
| n-Pr | Me | H | Me | CF₃ | Me | H | Cl | n-Pr | CF₃ |
| Me | Me | H | Me | C₂F₅ | Me | H | Cl | i-Pr | CF₃ |
| Et | Me | H | Me | C₂F₅ | Me | H | Cl | Cl | CF₃ |
| Me | Me | H | Et | CF₃ | Me | H | Cl | F | CF₃ |
| Me | Me | H | n-Pr | CF₃ | Me | H | Cl | Me | SMe |
| Me | Me | H | i-Pr | CF₃ | Me | H | Cl | Me | OMe |
| Me | Me | H | Cl | CF₃ | Me | H | Cl | Me | OEt |
| Me | Me | H | F | CF₃ | Me | H | Cl | Me | n-C₃F₇ |
| Me | Me | H | Me | SMe | Me | H | Cl | Me | i-C₃F₇ |
| Me | Me | H | Me | OMe | Me | H | Cl | Me | Et |
| Me | Me | H | Me | OEt | Me | H | Cl | Me | OCF₂CHF₂ |
| Me | Me | H | Me | n-C₃F₇ | Me | H | Cl | Me | SCF₂CHF₂ |
| Me | Me | H | Me | i-C₃F₇ | Me | H | Cl | Me | SO₂Me |
| Me | Me | H | Me | Et | Me | H | Cl | Me | SO₂CF₃ |
| Me | Me | H | Me | OCF₂CHF₂ | Me | H | Cl | CF₃ | CF₃ |
| Me | Me | H | Me | SCF₂CHF₂ | Me | H | Cl | CF₃ | Me |
| Me | Me | H | Me | SO₂Me | Me | H | Cl | OMe | CF₃ |
| Me | Me | H | Me | SO₂CF₃ | Me | H | Cl | H | CF₃ |
| Me | Me | H | CF₃ | CF₃ | Me | H | Cl | H | OCHF₂ |
| Me | Me | H | CF₃ | Me | Me | H | Cl | H | C₂F₅ |
| Me | Me | H | OMe | CF₃ | Et | H | Cl | H | C₂F₅ |
| Me | Me | H | H | CF₃ | Me | H | Cl | H | OCF₃ |
| Me | Me | H | H | OCHF₂ | Me | H | Cl | H | OCF₂CHF₂ |
| Me | Me | H | H | C₂F₅ | Me | H | Cl | H | SCF₂CHF₂ |
| Et | Me | H | H | C₂F₅ | Me | H | Cl | H | n-C₃F₇ |
| Me | Me | H | H | OCF₃ | Me | H | Cl | H | i-C₃F₇ |
| Me | Me | H | H | OCF₂CHF₂ | Me | H | Cl | H | Br |
| Me | Me | H | H | SCF₂CHF₂ | Me | H | Cl | H | Cl |
| Me | Me | H | H | n-C₃F₇ | Me | H | Cl | H | SCF₃ |
| Me | Me | H | H | i-C₃F₇ | Me | H | Cl | Ph | CF₃ |
| Me | Me | H | H | Br | Me | H | Cl | Ph | Cl |
| Me | Me | H | H | Cl | Me | H | Cl | Ph | Br |
| Me | Me | H | H | SCF₃ | Me | H | Cl | 2-pyridyl | CF₃ |
| Me | Me | H | Ph | CF₃ | Me | H | Cl | 2-pyridyl | Cl |
| Me | Me | H | Ph | Cl | Me | H | Cl | 2-ClPh | CF₃ |
| Me | Me | H | Ph | Br | Me | H | Cl | 2-ClPh | OCF₃ |
| Me | Me | H | 2-pyridyl | CF₃ | Me | H | Cl | 2-ClPh | Br |
| Me | Me | H | 2-pyridyl | Cl | Me | H | Cl | 2-ClPh | Cl |
| Me | Me | H | 2-ClPh | CF₃ | Me | H | Cl | 2-ClPh | SCHF₂ |
| Me | Me | H | 2-ClPh | OCF₃ | Me | H | Cl | 2-BrPh | CF₃ |
| Me | Me | H | 2-ClPh | Br | Me | H | Cl | 2-MePh | CF₃ |
| Me | Me | H | 2-ClPh | Cl | Me | H | Cl | 2-CNPh | CF₃ |
| Me | Me | H | 2-ClPh | SCHF₂ | Me | H | Cl | 2-FPh | CF₃ |
| Me | Me | H | 2-BrPh | CF₃ | Me | H | Cl | 2,6-F₂Ph | CF₃ |
| Me | Me | H | 2-MePh | CF₃ | Me | H | Cl | 2,4-F₂Ph | CF₃ |
| Me | Me | H | 2-CNPh | CF₃ | Me | H | Cl | 2,5-F₂Ph | CF₃ |
| Me | Me | H | 2-FPh | CF₃ | Me | H | Cl | 2-MeOPh | CF₃ |
| Me | Me | H | 2,6-F₂Ph | CF₃ | Me | H | Cl | 3-Cl-2-pyridyl | CF₃ |
| Me | Me | H | 2,4-F₂Ph | CF₃ | Me | H | Cl | 3-Cl-2-pyridyl | OCF₃ |
| Me | Me | H | 2,5-F₂Ph | CF₃ | Me | H | Cl | 3-Cl-2-pyridyl | Br |
| Me | Me | H | 2-MeOPh | CF₃ | Me | H | Cl | 3-Cl-2-pyridyl | Cl |
| Me | Me | H | 3-Cl-2-pyridyl | CF₃ | Me | H | Cl | 3-Cl-2-pyridyl | SCHF₂ |
| Me | Me | H | 3-Cl-2-pyridyl | OCF₃ | Me | H | Cl | 3-F-2-pyridyl | CF₃ |
| Me | Me | H | 3-CF₃-2- | CF₃ | Me | H | Cl | 3-CF₃-2- | CF₃ |
| | | | pyridyl | | | | | pyridyl | |
| Me | Me | H | 3-Me-2- | CF₃ | Me | H | Cl | 3-Me-2- | CF₃ |
| | | | pyridyl | | | | | pyridyl | |
| Me | Me | H | 3-Cl-2-pyridyl | SCHF₂ | Me | H | Cl | 3-Br-2-pyridyl | CF₃ |
| Me | Me | H | 3-F-2-pyridyl | CF₃ | Me | H | Cl | 3-Br-2-pyridyl | OCF₃ |
| Me | Me | H | 3-Cl-2-pyridyl | Br | Me | H | Cl | 3-Br-2-pyridyl | Br |
| Me | Me | H | 3-Cl-2-pyridyl | Cl | Me | H | Cl | 3-Br-2-pyridyl | Cl |
| Me | Me | H | 3-Br-2-pyridyl | CF₃ | Me | Cl | H | H | OCF₃ |
| Me | Me | H | 3-Br-2-pyridyl | OCF₃ | Me | Cl | H | H | OCF₂CHF₂ |
| Me | Me | H | 3-Br-2-pyridyl | Br | Me | Cl | H | H | SCF₂CHF₂ |
| Me | Me | H | 3-Br-2-pyridyl | Cl | Me | Cl | H | H | n-C₃F₇ |
| Me | Cl | H | Me | Br | Me | Cl | H | H | i-C₃F₇ |
| Me | Cl | H | Et | Br | Me | Cl | H | H | Br |
| Me | Cl | H | Me | Cl | Me | Cl | H | H | Cl |
| Me | Cl | H | Et | Cl | Me | Cl | H | H | SCF₃ |
| Me | Cl | H | Me | 1 | Me | Cl | H | Ph | CF₃ |
| Me | Cl | H | Me | CF₃ | Me | Cl | H | Ph | Cl |
| Me | Cl | H | Me | OCF₃ | Me | Cl | H | Ph | Br |
| Et | Cl | H | Me | CF₃ | Me | Cl | H | 2-pyridyl | CF₃ |
| Me | Cl | H | Me | SCF₃ | Me | Cl | H | 2-pyridyl | Cl |
| Me | Cl | H | Me | SCHF₂ | Me | Cl | H | 2-ClPh | CF₃ |
| Me | Cl | H | Me | OCHF₂ | Me | Cl | H | 2-ClPh | OCF₃ |
| n-Pr | Cl | H | Me | CF₃ | Me | Cl | H | 2-ClPh | Br |
| Me | Cl | H | Me | C₂F₅ | Me | Cl | H | 2-ClPh | Cl |
| Et | Cl | H | Me | C₂F₅ | Me | Cl | H | 2-ClPh | SCHF₂ |
| Me | Cl | H | El | CF₃ | Me | Cl | H | 2-BrPh | CF₃ |
| Me | Cl | H | n-Pr | CF₃ | Me | Cl | H | 2-MePh | CF₃ |
| Me | Cl | H | i-Pr | CF₃ | Me | Cl | H | 2-CNPh | CF₃ |
| Me | Cl | H | Cl | CF₃ | Me | Cl | H | 2-FPh | CF₃ |
| Me | Cl | H | F | CF₃ | Me | Cl | H | 2,6-F₂Ph | CF₃ |
| Me | Cl | H | Me | SMe | Me | Cl | H | 2,4-F₂Ph | CF₃ |
| Me | Cl | H | Me | OMe | Me | Cl | H | 2,5-F₂Ph | CF₃ |
| Me | Cl | H | Me | OEt | Me | Cl | H | 2-MeOPh | CF₃ |
| Me | Cl | H | Me | n-C₃F₇ | Me | Cl | H | 3-Cl-2-pyridyl | CF₃ |
| Me | Cl | H | Me | i-C₃F₇ | Me | Cl | H | 3-Cl-2-pyridyl | OCF₃ |
| Me | Cl | H | Me | Et | Me | Cl | H | 3-Cl-2-pyridyl | Br |
| Me | Cl | H | Me | OCF₂CHF₂ | Me | Cl | H | 3-Cl-2-pyridyl | Cl |
| Me | Cl | H | Me | SCF₂CHF₂ | Me | Cl | H | 3-Cl-2-pyridyl | SCHF₂ |
| Me | Cl | H | Me | SO₂Me | Me | Cl | H | 3-F-2-pyridyl | CF₃ |
| Me | Cl | H | Me | SO₂CF₃ | Me | Cl | H | CF₃ | CF₃ |
| Me | Cl | H | 3-CF₃-2- | CF₃ | Me | Cl | H | 3-Me-2- | CF₃ |
| | | | pyridyl | | | | | pyridyl | |
| Me | Cl | H | CF₃ | Me | Me | Cl | H | 3-Br-2-pyridyl | CF₃ |
| Me | Cl | H | OMe | CF₃ | Me | Cl | H | 3-Br-2-pyridyl | OCF₃ |
| Me | Cl | H | H | CF₃ | Me | Cl | H | 3-Br-2-pyridyl | Br |
| Me | Cl | H | H | OCHF₂ | Me | Cl | H | 3-Br-2-pyridyl | Cl |
| Me | Cl | H | H | C₂F₅ | Et | Cl | H | H | C₂F₅ |

TABLE 9

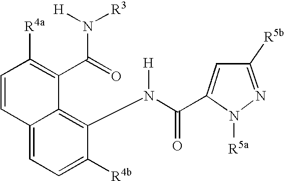

| R³ | R^{4a} | R^{4b} | R^{5a} | R^{5b} | R³ | R^{4a} | R^{4b} | R^{5a} | R^{5b} |
|---|---|---|---|---|---|---|---|---|---|
| Me | H | Me | H | CF₃ | Me | H | H | H | CF₃ |
| Me | H | Me | H | OCF₃ | Me | H | H | H | OCF₃ |
| Et | H | Me | H | OCF₃ | Et | H | H | H | OCF₃ |
| Me | H | Me | Me | Br | Me | H | H | Me | Br |
| Me | H | Me | Et | Br | Me | H | H | Et | Br |
| Me | H | Me | Me | Cl | Me | H | H | Me | Cl |
| Me | H | Me | Et | Cl | Me | H | H | Et | Cl |
| Me | H | Me | Me | I | Me | H | H | Me | I |
| Me | H | Me | Me | CF₃ | Me | H | H | Me | CF₃ |
| Me | H | Me | Me | OCF₃ | Me | H | H | Me | OCF₃ |
| Et | H | Me | Me | CF₃ | Et | H | H | Me | CF₃ |
| Me | H | Me | Me | SCF₃ | Me | H | H | Me | SCF₃ |
| Me | H | Me | Me | SCHF₂ | Me | H | H | Me | SCHF₂ |
| Me | H | Me | Me | OCHF₂ | Me | H | H | Me | OCHF₂ |
| n-Pr | H | Me | Me | CF₃ | n-Pr | H | H | Me | CF₃ |
| Me | H | Me | Me | C₂F₅ | Me | H | H | Me | C₂F₅ |
| Et | H | Me | Me | C₂F₅ | Et | H | H | Me | C₂F₅ |
| Me | H | Me | Et | CF₃ | Me | H | H | Et | CF₃ |
| Me | H | Me | n-Pr | CF₃ | Me | H | H | n-Pr | CF₃ |
| Me | H | Me | i-Pr | CF₃ | Me | H | H | i-Pr | CF₃ |
| Me | H | Me | i-Pr | OCF₃ | Me | H | H | Me | SMe |
| Me | H | Me | Me | SMe | Me | H | H | Me | OMe |
| Me | H | Me | Me | OMe | Me | H | H | Me | OEt |
| Me | H | Me | Me | OEt | Me | H | H | Me | n-C₃F₇ |
| Me | H | Me | Me | n-C₃F₇ | Me | H | H | Me | i-C₃F₇ |
| Me | H | Me | Me | i-C₃F₇ | Me | H | H | Me | Et |
| Me | H | Me | Me | Et | Me | H | H | Me | OCF₂CHF₂ |
| Me | H | Me | Me | OCF₂CHF₂ | Me | H | H | Me | SCF₂CHF₂ |
| Me | H | Me | Me | SCF₂CHF₂ | Me | H | H | Me | SO₂Me |
| Me | H | Me | Me | SO₂Me | Me | H | H | Me | SO₂CF₃ |
| Me | H | Me | Me | SO₂CF₃ | Me | H | H | CF₃ | CF₃ |
| Me | H | Me | CHF₂ | CF₃ | Me | H | H | CF₃ | Me |
| Me | H | Me | CHF₂ | Me | Me | H | H | Ph | CF₃ |
| Me | H | Me | Ph | CF₃ | Me | H | H | Ph | Cl |
| Me | H | Me | Ph | Cl | Me | H | H | Ph | Br |
| Me | H | Me | Ph | Br | Me | H | H | 2-pyridyl | CF₃ |
| Me | H | Me | 2-pyridyl | CF₃ | Me | H | H | 2-pyridyl | Cl |
| Me | H | Me | 2-pyridyl | Cl | Me | H | H | 2-ClPh | CF₃ |
| Me | H | Me | 2-ClPh | CF₃ | Me | H | H | 2-ClPh | OCF₃ |
| Me | H | Me | 2-ClPh | OCF₃ | Me | H | H | 2-ClPh | Br |
| Me | H | Me | 2-ClPh | Br | Me | H | H | 2-ClPh | Cl |
| Me | H | Me | 2-ClPh | Cl | Me | H | H | 2-ClPh | SCHF₂ |
| Me | H | Me | 2-ClPh | SCHF, | Me | H | H | 2-BrPh | CF₃ |
| Me | H | Me | 2-BrPh | CF₃ | Me | H | H | 2-MePh | CF₃ |
| Me | H | Me | 2-MePh | CF₃ | Me | H | H | 2-CNPh | CF₃ |
| Me | H | Me | 2-CNPh | CF₃ | Me | H | H | 2-FPh | CF₃ |
| Me | H | Me | 2-FPh | CF₃ | Me | H | H | 2,6-F₂Ph | CF₃ |
| Me | H | Me | 2,6-F₂Ph | CF₃ | Me | H | H | 2,4-F₂Ph | CF₃ |
| Me | H | Me | 2,4-F₂Ph | CF₃ | Me | H | H | 2,5-F₂Ph | CF₃ |
| Me | H | Me | 2,5-F₂Ph | CF₃ | Me | H | H | 2-MeOPh | CF₃ |
| Me | H | Me | 2-MeOPh | CF₃ | Me | H | H | 3-Cl-2-pyridyl | CF₃ |
| Me | H | Me | 3-Cl-2-pyridyl | CF₃ | Me | H | H | 3-Cl-2-pyridyl | OCF₃ |
| Me | H | Me | 3-Cl-2-pyridyl | OCF₃ | Me | H | H | 3-Cl-2-pyridyl | Br |
| Me | H | Me | 3-Cl-2-pyridyl | Br | Me | H | H | 3-Cl-2-pyridyl | Cl |
| Me | H | Me | 3-Cl-2-pyridyl | Cl | Me | H | H | 3-Cl-2-pyridyl | SCHF₂ |
| Me | H | Me | 3-Cl-2-pyridyl | SCHF₂ | Me | H | H | 3-F-2-pyridyl | CF₃ |
| Me | H | Me | 3-CF₃-2- | CF₃ | Me | H | H | 3-CF₃-2- | CF₃ |
| | | | pyridyl | | | | | pyridyl | |
| Me | H | Me | 3-Me-2- | CF₃ | Me | H | H | 3-Me-2- | CF₃ |
| | | | pyridyl | | | | | pyridyl | |
| Me | H | Me | 3-F-2-pyridyl | CF₃ | Me | H | H | 3-Br-2-pyridyl | CF₃ |
| Me | H | Me | 3-Br-2-pyridyl | CF₃ | Me | H | H | 3-Br-2-pyridyl | OCF₃ |
| Me | H | Me | 3-Br-2-pyridyl | OCF₃ | Me | H | H | 3-Br-2-pyridyl | Br |
| Me | H | Me | 3-Br-2-pyridyl | Br | Me | H | H | 3-Br-2-pyridyl | Cl |
| Me | H | Me | 3-Br-2-pyridyl | Cl | Me | H | Cl | Et | Br |
| Me | Me | H | Me | Br | Me | H | Cl | Me | Cl |
| Me | Me | H | Et | Br | Me | H | Cl | Et | Cl |
| Me | Me | H | Me | Cl | Me | H | Cl | Me | I |
| Me | Me | H | Et | Cl | Me | H | Cl | Me | CF₃ |
| Me | Me | H | Me | I | Me | H | Cl | Me | OCF₃ |
| Me | Me | H | Me | CF₃ | Et | H | Cl | Me | CF₃ |
| Me | Me | H | Me | OCF₃ | Me | H | Cl | Me | SCF₃ |
| Et | Me | H | Me | CF₃ | Me | H | Cl | Me | SCHF₂ |
| Me | Me | H | Me | SCF₃ | Me | H | Cl | Me | OCHF₂ |
| Me | Me | H | Me | SCHF₂ | n-Pr | H | Cl | Me | CF₃ |
| Me | Me | H | Me | OCHF₂ | Me | H | Cl | Me | C₂F₅ |
| n-Pr | Me | H | Me | CF₃ | Et | H | Cl | Me | C₂F₅ |
| Me | Me | H | Me | C₂F₅ | Me | H | Cl | Et | CF₃ |
| Et | Me | H | Me | C₂F₅ | Me | H | Cl | n-Pr | CF₃ |
| Me | Me | H | Et | CF₃ | Me | H | Cl | i-Pr | CF₃ |
| Me | Me | H | n-Pr | CF₃ | Me | H | Cl | Me | SMe |
| Me | Me | H | i-Pr | CF₃ | Me | H | Cl | Me | OMe |
| Me | Me | H | Me | SMe | Me | H | Cl | Me | OEt |
| Me | Me | H | Me | OMe | Me | H | Cl | Me | n-C₃F₇ |
| Me | Me | H | Me | OEt | Me | H | Cl | Me | i-C₃F₇ |
| Me | Me | H | Me | n-C₃F₇ | Me | H | Cl | Me | Et |
| Me | Me | H | Me | i-C₃F₇ | Me | H | Cl | Me | OCF₂CHF₂ |
| Me | Me | H | Me | Et | Me | H | Cl | Me | SCF₂CHF₂ |
| Me | Me | H | Me | OCF₂CHF₂ | Me | H | Cl | Me | SO₂Me |
| Me | Me | H | Me | SCF₂CHF₂ | Me | H | Cl | Me | SO₂CF₃ |
| Me | Me | H | Me | SO₂Me | Me | H | Cl | CF₃ | CF₃ |
| Me | Me | H | Me | SO₂CF₃ | Me | H | Cl | CF₃ | Me |
| Me | Me | H | CF₃ | CF₃ | Me | H | Cl | Ph | CF₃ |
| Me | Me | H | CF₃ | Me | Me | H | Cl | Ph | Cl |
| Me | Me | H | Ph | CF₃ | Me | H | Cl | Ph | Br |
| Me | Me | H | Ph | Cl | Me | H | Cl | 2-pyridyl | CF₃ |
| Me | Me | H | Ph | Br | Me | H | Cl | 2-pyridyl | Cl |
| Me | Me | H | 2-pyridyl | CF₃ | Me | H | Cl | 2-ClPh | CF₃ |
| Me | Me | H | 2-pyridyl | Cl | Me | H | Cl | 2-ClPh | OCF₃ |
| Me | Me | H | 2-ClPh | CF₃ | Me | H | Cl | 2-ClPh | Br |
| Me | Me | H | 2-ClPh | OCF₃ | Me | H | Cl | 2-ClPh | Cl |
| Me | Me | H | 2-ClPh | Br | Me | H | Cl | 2-ClPh | SCHF₂ |
| Me | Me | H | 2-ClPh | Cl | Me | H | Cl | 2-BrPh | CF₃ |
| Me | Me | H | 2-ClPh | SCHF₂ | Me | H | Cl | 2-MePh | CF₃ |
| Me | Me | H | 2-BrPh | CF₃ | Me | H | Cl | 2-CNPh | CF₃ |
| Me | Me | H | 2-MePh | CF₃ | Me | H | Cl | 2-FPh | CF₃ |
| Me | Me | H | 2-CNPh | CF₃ | Me | H | Cl | 2,6-F₂Ph | CF₃ |
| Me | Me | H | 2-FPh | CF₃ | Me | H | Cl | 2,4-F₂Ph | CF₃ |
| Me | Me | H | 2,6-F₂Ph | CF₃ | Me | H | Cl | 2,5-F₂Ph | CF₃ |
| Me | Me | H | 2,4-F₂Ph | CF₃ | Me | H | Cl | 2-OMe | CF₃ |
| Me | Me | H | 2,5-F₂Ph | CF₃ | Me | H | Cl | 3-Cl-2-pyridyl | CF₃ |
| Me | Me | H | 2-MeOPh | CF₃ | Me | H | Cl | 3-Cl-2-pyridyl | OCF₃ |
| Me | Me | H | 3-Cl-2-pyridyl | CF₃ | Me | H | Cl | 3-Cl-2-pyridyl | Br |
| Me | Me | H | 3-Cl-2-pyridyl | OCF₃ | Me | H | Cl | 3-Cl-2-pyridyl | Cl |
| Me | Me | H | 3-Cl-2-pyridyl | Br | Me | H | Cl | 3-Cl-2-pyridyl | SCHF₂ |
| Me | Me | H | 3-Cl-2-pyridyl | Cl | Me | H | Cl | 3-F-2-pyridyl | CF₃ |
| Me | Me | H | 3-CF₃-2- | CF₃ | Me | H | Cl | 3-CF₃-2- | CF₃ |
| | | | pyridyl | | | | | pyridyl | |
| Me | Me | H | 3-Me-2- | CF₃ | Me | H | Cl | 3-Me-2- | CF₃ |
| | | | pyridyl | | | | | pyridyl | |
| Me | Me | H | 3-Cl-2-pyridyl | SCHF₂ | Me | H | Cl | 3-Br-2-pyridyl | CF₃ |
| Me | Me | H | 3-F-2-pyridyl | CF₃ | Me | H | Cl | 3-Br-2-pyridyl | OCF₃ |
| Me | Me | H | 3-Br-2-pyridyl | CF₃ | Me | H | Cl | 3-Br-2-pyridyl | Br |
| Me | Me | H | 3-Br-2-pyridyl | OCF₃ | Me | H | Cl | 3-Br-2-pyridyl | Cl |
| Me | Me | H | 3-Br-2-pyridyl | Br | Me | Cl | H | Et | CF₃ |
| Me | Me | H | 3-Br-2-pyridyl | Cl | Me | Cl | H | n-Pr | CF₃ |
| Me | Cl | H | Me | Br | Me | Cl | H | i-Pr | CF₃ |
| Me | Cl | H | Et | Br | Me | Cl | H | Me | SMe |
| Me | Cl | H | Me | Cl | Me | Cl | H | Me | OMe |
| Me | Cl | H | Et | Cl | Me | Cl | H | Me | OEt |
| Me | Cl | H | Me | I | Me | Cl | H | Me | n-C₃F₇ |
| Me | Cl | H | Me | CF₃ | Me | Cl | H | Me | i-C₃F₇ |
| Me | Cl | H | Me | OCF₃ | Me | Cl | H | Me | Et |
| Et | Cl | H | Me | CF₃ | Me | Cl | H | Me | OCF₂CHF₂ |
| Me | Cl | H | Me | SCF₃ | Me | Cl | H | Me | SCF₂CHF₂ |
| Me | Cl | H | Me | SCHF₂ | Me | Cl | H | Me | SO₂Me |
| Me | Cl | H | Me | OCHF₂ | Me | Cl | H | Me | SO₂CF₃ |
| n-Pr | Cl | H | Me | CF₃ | Me | Cl | H | CF₃ | CF₃ |
| Me | Cl | H | Me | C₂F₅ | Me | Cl | H | CF₃ | Me |
| Et | Cl | H | Me | C₂F₅ | Me | Cl | H | OMe | CF₃ |
| Me | Cl | H | 3-Cl-2-pyridyl | CF₃ | Me | Cl | H | Ph | CF₃ |
| Me | Cl | H | 3-Cl-2-pyridyl | OCF₃ | Me | Cl | H | Ph | Cl |
| Me | Cl | H | 3-Cl-2-pyridyl | Br | Me | Cl | H | Ph | Br |
| Me | Cl | H | 3-Cl-2-pyridyl | Cl | Me | Cl | H | 2-pyridyl | CF₃ |
| Me | Cl | H | 3-Cl-2-pyridyl | SCHF₂ | Me | Cl | H | 2-pyridyl | Cl |
| Me | Cl | H | 3-F-2-pyridyl | CF₃ | Me | Cl | H | 2-ClPh | CF₃ |
| Me | Cl | H | 3-CF₃-2- | CF₃ | Me | Cl | H | 3-Me-2- | CF₃ |
| | | | pyridyl | | | | | pyridyl | |
| Me | Cl | H | 3-Br-2-pyridyl | CF₃ | Me | Cl | H | 2-ClPh | Br |
| Me | Cl | H | 3-Br-2-pyridyl | OCF₃ | Me | Cl | H | 2-ClPh | Cl |
| Me | Cl | H | 3-Br-2-pyridyl | Br | Me | Cl | H | 2-ClPh | SCHF₂ |
| Me | Cl | H | 3-Br-2-pyridyl | Cl | Me | Cl | H | 2-BrPh | CF₃ |
| Me | Cl | H | 2,4-F₂Ph | CF₃ | Me | Cl | H | 2-MePh | CF₃ |
| Me | Cl | H | 2,5-F₂Ph | CF₃ | Me | Cl | H | 2-CNPh | CF₃ |
| Me | Cl | H | 2-OMe | CF₃ | Me | Cl | H | 2-FPh | CF₃ |
| Me | Cl | H | 2-ClPh | OCF₃ | Me | Cl | H | 2,6-F₂Ph | CF₃ |

TABLE 10

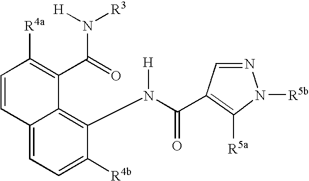

| R³ | R^{4a} | R^{4b} | R^{5a} | R^{5b} | R³ | R^{4a} | R^{4b} | R^{5a} | R^{5b} |
|---|---|---|---|---|---|---|---|---|---|
| Me | H | Me | H | CHF₂ | Me | H | H | H | CHF₂ |
| Me | H | Me | H | CH₂CF₃ | Me | H | H | H | CH₂CF₃ |
| Et | H | Me | H | CH₂CF₃ | Et | H | H | H | CH₂CF₃ |
| Me | H | Me | Me | CH₂CF₃ | Me | H | H | Me | CH₂CF₃ |
| Me | H | Me | Et | CH₂CF₃ | Me | H | H | Et | CH₂CF₃ |
| Me | H | Me | Me | CF₂CHF₂ | Me | H | H | Me | CF₂CHF₂ |
| Me | H | Me | Et | CHF₂ | Me | H | H | Et | CHF₂ |
| Me | H | Me | Me | CHF₂ | Me | H | H | Me | CHF₂ |
| Me | H | Me | Me | CBrF₂ | Me | H | H | Me | CBrF₂ |
| Me | H | Me | Me | CHF₂ | Me | H | H | Me | CHF₂ |
| Et | H | Me | Me | CH₂CF₃ | Et | H | H | Me | CH₂CF₃ |
| Me | H | Me | Me | Et | Me | H | H | Me | Et |
| Me | H | Me | Me | n-Pr | Me | H | H | Me | n-Pr |
| Me | H | Me | Me | CH₂C₂F₅ | Me | H | H | Me | CH₂C₂F₅ |
| n-Pr | H | Me | Me | CH₂CF₃ | n-Pr | H | H | Me | CH₂CF₃ |
| Me | H | Me | Me | CF₃ | Me | H | H | Me | CF₃ |
| Et | H | Me | Me | C₂F₅ | Et | H | H | Me | C₂F₅ |
| Me | H | Me | Et | CHF₂ | Me | H | H | Et | CHF₂ |
| Me | H | Me | n-Pr | CH₂CF₃ | Me | H | H | n-Pr | CH₂CF₃ |
| Me | H | Me | i-Pr | CHF₂ | Me | H | H | i-Pr | CHF₂ |
| Me | H | Me | Cl | CH₂CF₃ | Me | H | H | Cl | CH₂CF₃ |
| Me | H | Me | F | CH₂CF₃ | Me | H | H | F | CH₂CF₃ |
| Me | H | Me | Me | CH₂Cl | Me | H | H | Me | CH₂Cl |
| Me | H | Me | Me | CClF₂ | Me | H | H | Me | CClF₂ |
| Me | H | Me | Me | CH₂CH₂Cl | Me | H | H | Me | CH₂CH₂C1 |
| Me | H | Me | Me | n-C₃F₇ | Me | H | H | Me | n-C₃F₇ |
| Me | H | Me | Me | i-C₃F₇ | Me | H | H | Me | i-C₃F₇ |
| Me | H | Me | Me | Allyl | Me | H | H | Me | Allyl |
| Me | H | Me | Et | CF₂CHF₂ | Me | H | H | Me | CF₂CHF₂ |
| Me | H | Me | Et | i-C₃F₇ | Me | H | H | Me | i-C₃F₇ |
| Me | H | Me | i-Pr | CF₂CHF₂ | Me | H | H | Me | CF₂CHF₂ |
| Me | H | Me | n-Pr | CF₂CHF₂ | Me | H | H | Me | CF₂CHF₂ |
| Me | H | Me | CF₃ | CF₂CHF₂ | Me | H | H | CF₃ | CF₂CHF₂ |
| Me | H | Me | CF₃ | Me | Me | H | H | CF₃ | Me |
| Me | H | Me | OMe | CH₂CF₃ | Me | H | H | OMe | CH₂CF₃ |
| Me | H | Me | H | CH₂CF₃ | Me | H | H | H | CH₂CF₃ |
| Me | H | Me | H | CH₂CF₃ | Me | H | H | H | CH₂CF₃ |
| Me | H | Me | H | C₂F₅ | Me | H | H | H | C₂F₅ |
| Et | H | Me | H | C₂F₅ | Et | H | H | H | C₂F₅ |
| Me | H | Me | H | C₂F₅ | Me | H | H | H | C₂F₅ |
| Me | H | Me | H | CF₂CHF₂ | Me | H | H | H | CF₂CHF₂ |
| Me | H | Me | i-Pr | CH₂CF₃ | Me | H | H | H | CH₂CF₃ |
| Me | H | Me | H | n-C₃F₇ | Me | H | H | H | n-C₃F₇ |
| Me | H | Me | H | i-C₃F₇ | Me | H | H | H | i-C₃F₇ |
| Me | H | Me | Ph | CH₂CF₃ | Me | H | H | H | CH₂CF₃ |
| Me | H | Me | Ph | CF₂CHF₂ | Me | H | H | H | CF₂CHF₂ |
| Me | H | Me | Ph | CHF₂ | Me | H | H | H | CHF₂ |
| Me | H | Me | 2-pyridyl | CH₂CF₃ | Me | H | H | Ph | CH₂CF₃ |
| Me | H | Me | 2-pyridyl | CF₂CHF₂ | Me | H | H | Ph | CF₂CHF₂ |
| Me | H | Me | 2-ClPh | CH₂CF₃ | Me | H | H | Ph | CH₂CF₃ |
| Me | H | Me | 2-ClPh | CF₂CHF₂ | Me | H | H | 2-pyridyl | CF₂CHF₂ |
| Me | H | Me | 2-ClPh | CHF₂ | Me | H | H | 2-pyridyl | CHF₂ |
| Me | H | Me | 2-ClPh | Et | Me | H | H | 2-ClPh | Et |
| Me | H | Me | 2-ClPh | CBrF₂ | Me | H | H | 2-ClPh | CBrF₂ |
| Me | H | Me | 2-BrPh | CH₂CF₃ | Me | H | H | 2-ClPh | CH₂CF₃ |
| Me | H | Me | 2-MePh | CH₂CF₃ | Me | H | H | 2-ClPh | CH₂CF₃ |
| Me | H | Me | 2-CNPh | CH₂CF₃ | Me | H | H | 2-ClPh | CH₂CF₃ |
| Me | H | Me | 2-FPh | CH₂CF₃ | Me | H | H | 2-BrPh | CH₂CF₃ |
| Me | H | Me | 2,6-F₂Ph | CH₂CF₃ | Me | H | H | 2-MePh | CH₂CF₃ |
| Me | H | Me | 2,4-F₂Ph | CH₂CF₃ | Me | H | H | 2-CNPh | CH₂CF₃ |
| Me | H | Me | 2,5-F₂Ph | CH₂CF₃ | Me | H | H | 2-FPh | CH₂CF₃ |
| Me | H | Me | 2-MeOPh | CH₂CF₃ | Me | H | H | 2,6-F₂Ph | CH₂CF₃ |
| Me | H | Me | 3-Cl-2-pyridyl | CH₂CF₃ | Me | H | H | 2,4-F₂Ph | CH₂CF₃ |
| Me | H | Me | 3-Cl-2-pyridyl | CF₂CHF₂ | Me | H | H | 2,5-F-₂Ph | CF₂CHF₂ |
| Me | H | Me | 3-Cl-2-pyridyl | CF₃ | Me | H | H | 2-MeOPh | CF₃ |
| Me | H | Me | 3-Cl-2-pyridyl | CHF₂ | Me | H | H | 3-Cl-2-pyridyl | CHF₂ |
| Me | H | Me | 3-Cl-2-pyridyl | CBrF₂ | Me | H | H | 3-Cl-2-pyridyl | CBrF₂ |
| Me | H | Me | 3-F-2-pyridyl | CH₂CF₃ | Me | H | H | 3-Cl-2-pyridyl | CH₂CF₃ |
| Me | H | Me | 3-CF₃-2- | CH₂CF₃ | Me | H | H | 3-CF₃-2- | CH₂CF₃ |
| | | | pyridyl | | | | | pyridyl | |
| Me | H | Me | 3-Me-2- | CH₂CF₃ | Me | H | H | 3-Me-2- | CF₂CHF₂ |
| | | | pyridyl | | | | | pyridyl | |
| Me | H | Me | 3-Br-2-pyridyl | CF₃ | Me | H | H | 3-Cl-2-pyridyl | CH₂CF₃ |
| Me | H | Me | 3-Br-2-pyridyl | CH₂CF₃ | Me | H | H | 3-Cl-2-pyridyl | CH₂CF₃ |
| Me | H | Me | 3-Br-2-pyridyl | CF₂CHF₂ | Me | H | H | 3-F-2-pyridyl | CF₃ |
| Me | H | Me | 3-Br-2-pyridyl | CClF₂ | Me | H | H | 3-Br-2-pyridyl | CClF₂ |
| Me | Me | H | H | CHF₂ | Me | H | H | 3-Br-2-pyridyl | CH₂CF₃ |
| Me | Me | H | H | CH₂CF₃ | Me | H | H | 3-Br-2-pyridyl | CF₃ |
| Et | Me | H | H | CH₂CF₃ | Me | H | H | 3-Br-2-pyridyl | CF₃ |
| Me | Me | H | Me | CH₂CF₃ | Me | H | Cl | Et | CHF₂ |
| Me | Me | H | Et | CH₂CF₃ | Me | H | Cl | Me | CH₂CF₃ |
| Me | Me | H | Me | CF₂CHF₂ | Me | H | Cl | Et | CH₂CF₃ |
| Me | Me | H | Et | CHF₂ | Me | H | Cl | Me | CH₂CF₃ |
| Me | Me | H | Me | CHF₂ | Me | H | Cl | Me | CH₂CF₃ |
| Me | Me | H | Me | CBrF₂ | Me | H | Cl | Me | CF₂CHF₂ |
| Me | Me | H | Me | CHF₂ | Et | H | Cl | Me | CHF₂ |
| Et | Me | H | Me | CH₂CF₃ | Me | H | Cl | Me | CHF₂ |
| Me | Me | H | Me | Et | Me | H | Cl | Me | CBrF₂ |
| Me | Me | H | Me | n-Pr | Me | H | Cl | Me | CHF₂ |
| Me | Me | H | Me | CH₂C₂F₅ | n-Pr | H | Cl | Me | CH₂CF₃ |
| n-Pr | Me | H | Me | CH₂CF₃ | Me | H | Cl | Me | Et |
| Me | Me | H | Me | CF₃ | Et | H | Cl | Me | n-Pr |
| Et | Me | H | Me | C₂F₅ | Me | H | Cl | Et | CH₂C₂F₅ |
| Me | Me | H | Et | CHF₂ | Me | H | Cl | n-Pr | CH₂CF₃ |
| Me | Me | H | n-Pr | CH₂CF₃ | Me | H | Cl | i-Pr | CF₃ |
| Me | Me | H | i-Pr | CHF₂ | Me | H | Cl | Cl | C₂F₅ |
| Me | Me | H | Cl | CH₂CF₃ | Me | H | Cl | F | CHF₂ |
| Me | Me | H | F | CH₂CF₃ | Me | H | Cl | Me | CH₂CF₃ |
| Me | Me | H | Me | CH₂Cl | Me | H | Cl | Me | CHF₂ |
| Me | Me | H | Me | CClF₂ | Me | H | Cl | Me | CH₂CF₃ |
| Me | Me | H | Me | CH₂CH₂Cl | Me | H | Cl | Me | CH₂CF₃ |
| Me | Me | H | Me | n-C₃F₇ | Me | H | Cl | Me | CH₂Cl |
| Me | Me | H | Me | i-C₃F₇ | Me | H | Cl | Me | CClF₂ |
| Me | Me | H | Me | Allyl | Me | H | Cl | Me | CH₂CH₂Cl |
| Me | Me | H | Me | CF₂CHF₂ | Me | H | Cl | Me | n-C₃F₇ |
| Me | Me | H | Me | i-C₃F₇ | Me | H | Cl | Me | i-C₃F₇ |
| Me | Me | H | Me | CF₂CHF₂ | Me | H | Cl | Me | Allyl |
| Me | Me | H | Me | CF₂CHF₂ | Me | H | Cl | CF₃ | CF₂CHF₂ |
| Me | Me | H | CF₃ | CF₂CHF₂ | Me | H | Cl | CF₃ | i-C₃F₇ |
| Me | Me | H | CF₃ | Me | Me | H | Cl | OMe | CF₂CHF₂ |
| Me | Me | H | OMe | CH₂CF₃ | Me | H | Cl | H | CF₂CHF₂ |
| Me | Me | H | H | CH₂CF₃ | Me | H | Cl | H | CF₂CHF₂ |
| Me | Me | H | H | CH₂CF₃ | Me | H | Cl | H | Me |
| Me | Me | H | H | C₂F₅ | Et | H | Cl | H | CH₂CF₃ |
| Et | Me | H | H | C₂F₅ | Me | H | Cl | H | CH₂CF₃ |
| Me | Me | H | H | C₂F₅ | Me | H | Cl | H | CH₂CF₃ |
| Me | Me | H | H | CF₂CHF₂ | Me | H | Cl | H | C₂F₅ |
| Me | Me | H | H | CH₂CF₃ | Me | H | Cl | H | C₂F₅ |
| Me | Me | H | H | n-C₃F₇ | Me | H | Cl | H | C₂F₅ |
| Me | Me | H | H | i-C₃F₇ | Me | H | Cl | H | CF₂CHF₂ |
| Me | Me | H | H | CH₂CF₃ | Me | H | Cl | H | CH₂CF₃ |
| Me | Me | H | H | CF₂CHF₂ | Me | H | Cl | H | n-C₃F₇ |
| Me | Me | H | H | CHF₂ | Me | H | Cl | Ph | i-C₃F₇ |
| Me | Me | H | Ph | CH₂CF₃ | Me | H | Cl | Ph | CH₂CF₃ |
| Me | Me | H | Ph | CF₂CHF₂ | Me | H | Cl | Ph | CF₂CHF₂ |
| Me | Me | H | Ph | CH₂CF₃ | Me | H | Cl | 2-pyridyl | CHF₂ |
| Me | Me | H | 2-pyridyl | CF₂CHF₂ | Me | H | Cl | 2-pyridyl | CH₂CF₃ |
| Me | Me | H | 2-pyridyl | CHF₂ | Me | H | Cl | 2-ClPh | CF₂CHF₂ |
| Me | Me | H | 2-ClPh | Et | Me | H | Cl | 2-ClPh | CH₂CF₃ |
| Me | Me | H | 2-ClPh | CBrF₂ | Me | H | Cl | 2-ClPh | CF₂CHF₂ |
| Me | Me | H | 2-ClPh | CH₂CF₃ | Me | H | Cl | 2-ClPh | CHF₂ |
| Me | Me | H | 2-ClPh | CH₂CF₃ | Me | H | Cl | 2-ClPh | Et |
| Me | Me | H | 2-ClPh | CH₂CF₃ | Me | H | Cl | 2-BrPh | CBrF₂ |
| Me | Me | H | 2-BrPh | CH₂CF₃ | Me | H | Cl | 2-MePh | CH₂CF₃ |
| Me | Me | H | 2-MePh | CH₂CF₃ | Me | H | Cl | 2-CNPh | CH₂CF₃ |
| Me | Me | H | 2-CNPh | CH₂CF₃ | Me | H | Cl | 2-FPh | CH₂CF₃ |
| Me | Me | H | 2-FPh | CH₂CF₃ | Me | H | Cl | 2,6-F₂Ph | CH₂CF₃ |
| Me | Me | H | 2,6-F₂Ph | CH₂CF₃ | Me | H | Cl | 2,4-F₂Ph | CH₂CF₃ |
| Me | Me | H | 2,4-F₂Ph | CH₂CF₃ | Me | H | Cl | 2,5-F₂Ph | CH₂CF₃ |
| Me | Me | H | 2,5-F₂Ph | CF₂CHF₂ | Me | H | Cl | 2-MeOPh | CH₂CF₃ |
| Me | Me | H | 2-MeOPh | CF₃ | Me | H | Cl | 3-Cl-2-pyridyl | CH₂CF₃ |
| Me | Me | H | 3-Cl-2-pyridyl | CHF₂ | Me | H | Cl | 3-Cl-2-pyridyl | CH₂CF₃ |
| Me | Me | H | 3-Cl-2-pyridyl | CBrF₂ | Me | H | Cl | 3-Cl-2-pyridyl | CF₂CHF₂ |
| Me | Me | H | 3-Cl-2-pyridyl | CH₂CF₃ | Me | H | Cl | 3-Cl-2-pyridyl | CF₃ |
| Me | Me | H | 3-Cl-2-pyridyl | CH₂CF₃ | Me | H | Cl | 3-Cl-2-pyridyl | CHF₂ |
| Me | Me | H | 3-Cl-2-pyridyl | CH₂CF₃ | Me | H | Cl | 3-F-2-pyridyl | CBrF₂ |
| Me | Me | H | 3-CF₃-2- | CH₂CF₃ | Me | H | Cl | 3-CF₃-2- | CH₂CF₃ |
| | | | pyridyl | pyridyl | | | | | |
| Me | Me | H | 3-Me-2- | CF₂CHF₂ | Me | H | Cl | 3-Me-2- | CH₂CF₃ |
| | | | pyridyl | | | | | pyridyl | |
| Me | Me | H | 3-F-2-pyridyl | CF₃ | Me | H | Cl | 3-Br-2-pyridyl | CH₂CF₃ |
| Me | Me | H | 3-Br-2-pyridyl | CClF₂ | Me | H | Cl | 3-Br-2-pyridyl | CF₃ |
| Me | Me | H | 3-Br-2-pyridyl | CH₂CF₃ | Me | H | Cl | 3-Br-2-pyridyl | CH₂CF₃ |
| Me | Me | H | 3-Br-2-pyridyl | CF₃ | Me | H | Cl | 3-Br-2-pyridyl | CF₂CHF₂ |
| Me | Me | H | 3-Br-2-pyridyl | CF₃ | Me | Cl | H | H | CF₂CHF₂ |
| Me | Cl | H | Me | CHF₂ | Me | Cl | H | H | CH₂CF₃ |
| Me | Cl | H | Et | CH₂CF₃ | Me | Cl | H | H | n-C₃F₇ |
| Me | Cl | H | Me | CH₂CF₃ | Me | Cl | H | H | i-C₃F₇ |
| Me | Cl | H | Et | CH₂CF₃ | Me | Cl | H | Ph | CH₂CF₃ |
| Me | Cl | H | Me | CH₂CF₃ | Me | Cl | H | Ph | CF₂CHF₂ |
| Me | Cl | H | Me | CF₂CHF₂ | Me | Cl | H | Ph | CHF₂ |
| Me | Cl | H | Me | CHF₂ | Me | Cl | H | 2-pyridyl | CH₂CF₃ |
| Et | Cl | H | Me | CHF₂ | Me | Cl | H | 2-pyridyl | CF₂CHF₂ |
| Me | Cl | H | Me | CBrF₂ | Me | Cl | H | 2-ClPh | CH₂CF₃ |
| Me | Cl | H | Me | CHF₂ | Me | Cl | H | 2-ClPh | CF₂CHF₂ |
| Me | Cl | H | Me | CH₂CF₃ | Me | Cl | H | 2-ClPh | CHF₂ |
| n-Pr | Cl | H | Me | Et | Me | Cl | H | 2-ClPh | Et |
| Me | Cl | H | Me | n-Pr | Me | Cl | H | 2-ClPh | CBrF₂ |
| Et | Cl | H | Me | CH₂C₂F₅ | Me | Cl | H | 2-BrPh | CH₂CF₃ |
| Me | Cl | H | Et | CH₂CF₃ | Me | Cl | H | 2-MePh | CH₂CF₃ |
| Me | Cl | H | n-Pr | CF₃ | Me | Cl | H | 2-CNPh | CH₂CF₃ |
| Me | Cl | H | i-Pr | C₂F₅ | Me | Cl | H | 2-FPh | CH₂CF₃ |
| Me | Cl | H | Cl | CHF₂ | Me | Cl | H | 2,6-F₂Ph | CH₂CF₃ |
| Me | Cl | H | F | CH₂CF₃ | Me | Cl | H | 2,4-F₂Ph | CH₂CF₃ |
| Me | Cl | H | Me | CHF₂ | Me | Cl | H | 2,5-F₂Ph | CH₂CF₃ |
| Me | Cl | H | Me | CH₂CF₃ | Me | Cl | H | 2-MeOPh | CH₂CF₃ |
| Me | Cl | H | Me | CH₂CF₃ | Me | Cl | H | 3-Cl-2-pyridyl | CH₂CF₃ |
| Me | Cl | H | Me | CH₂Cl | Me | Cl | H | 3-Cl-2-pyridyl | CF₂CHF₂ |
| Me | Cl | H | Me | CClF₂ | Me | Cl | H | 3-Cl-2-pyridyl | CF₃ |
| Me | Cl | H | Me | CH₂CH₂Cl | Me | Cl | H | 3-Cl-2-pyridyl | CHF₂ |
| Me | Cl | H | Me | n-C₃F₇ | Me | Cl | H | 3-Cl-2-pyridyl | CBrF₂ |
| Me | Cl | H | Me | i-C₃F₇ | Me | Cl | H | 3-F-2-pyridyl | CH₂CF₃ |
| Me | Cl | H | 3-Me-2- | CH₂CF₃ | Me | Cl | H | 3-CF₃-2- | CH₂CF₃ |
| | | | pyridyl | | | | pyridyl | | |
| Me | Cl | H | Me | CF₂CHF₂ | Me | Cl | H | 3-Br-2-pyridyl | CF₃ |
| Me | Cl | H | CF₃ | i-C₃F₇ | Me | Cl | H | 3-Br-2-pyridyl | CH₂CF₃ |
| Me | Cl | H | CF₃ | CF₂CHF₂ | Me | Cl | H | 3-Br-2-pyridyl | CF₂CHF₂ |
| Me | Cl | H | OMe | CF₂CHF₂ | Me | Cl | H | 3-Br-2-pyridyl | CClF₂ |
| Me | Cl | H | H | CF₂CHF₂ | Me | Cl | H | H | C₂F₅ |
| Me | Cl | H | H | Me | Me | Cl | H | H | C₂F₅ |
| Me | Cl | H | H | CH₂CF₃ | Me | Cl | H | H | C₂F₅ |
| Et | Cl | H | H | CH₂CF₃ | Me | Cl | H | Me | Allyl |
| Me | Cl | H | H | CH₂CF₃ | | | | | |

Formulation/Utility

Compounds of this invention will generally be used as a formulation or composition with an agriculturally suitable carrier comprising at least one of a liquid diluent, a solid diluent or a surfactant. The formulation or composition ingredients are selected to be consistent with the physical properties of the active ingredient, mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and Surfactant within the following approximate ranges that add up to 100 percent by weight.

| | Weight Percent | | |
|---|---|---|---|
| | Active | | |
| | Ingredient | Diluent | Surfactant |
| Water-Dispersible and Water-soluble | 5-90 | 0-94 | 1-15 |
| Granules, Tablets and Powders. | | | |
| Suspensions, Emulsions, Solutions | 5-50 | 40-95 | 0-15 |
| (including Emulsifiable | | | |
| Concentrates) | | | |
| Dusts | 1-25 | 70-99 | 0-5 |
| Granules and Pellets | 0.01- | 5- | 0-15 |
| | 99 | 99.99 | |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Detergents and Emulsifiers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, and polyoxyethylene/polyoxypropylene block copolymers. Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering Dec. 4, 1967, pp 147-48, Perry's Chemical Engineer's Handbook, 4th Ed., McGraw-Hill, New York, 1963, pages 8-57 and following, and PCT Publication WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscienice, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 14; Klingman, Weed Control as a Science, John Wiley and Sons, Inc., New York, 1961, pp 81-96; and Hance et al., Weed Control Handbook, 8th Ed., Blackwell Scientific Publications, Oxford, 1989.

In the following Examples, all percentages are by weight and all formulations are prepared in conventional ways. Compound numbers refer to compounds in Index Table A.

### EXAMPLE A

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### EXAMPLE B

Granule

| | |
|---|---|
| Compound 7 | 10.0% |
| attapulgite granules (low volatile matter, | |
| 0.71/0.30 mm; U.S.S. No. 25-50 sieves) | 90.0%. |

### EXAMPLE C

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### EXAMPLE D

Emulsifiable Concentrate

| | |
|---|---|
| Compound 7 | 20.0% |
| blend of oil soluble sulfonates | |
| and polyoxyethylene ethers | 10.0% |
| isophorone | 70.0%. |

### EXAMPLE E

Granule

| | |
|---|---|
| Compound 1 | 0.5% |
| cellulose | 2.5% |
| lactose | 4.0% |
| cornmeal | 93.0%. |

Compounds of this invention are characterized by favorable metabolic and/or soil residual patterns and exhibit activity controlling a spectrum of agronomic and non-agronomic invertebrate pests. (In the context of this disclosure “invertebrate pest control” means inhibition of invertebrate pest development (including mortality) that causes significant reduction in feeding or other injury or damage caused by the pest; related expressions are defined analogously.) As referred to in this disclosure, the term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” includes all of the helminths, such as: roundworms, heartworms, and phytophagous nematodes (Nematoda), flukes (Tematoda), Acanthocephala, and tapeworms (Cestoda). Those skilled in the art will recognize that not all compounds are equally effective against all pests. Compounds of this invention display activity against economically important agronomic, forest, greenhouse, nursery, ornamentals, food and fiber, public and animal health, domestic and commercial structure, household, and stored product pests. These include larvae of the order Lepidoptera, such as armyworms, cutworms, loopers, and heliothines in the family Noctuidae (e.g., fall armyworm (Spodoptera fugiperda J. E. Smith), beet armyworm (Spodoptera exigua Hübner), black cutworm (Agrotis ipsilon Hufnagel), cabbage looper (Trichoplusia ni Hübner), tobacco budworm (Heliothis virescens Fabricius)); borers, casebearers, webworms, coneworms, cabbageworms and skeletonizers from the family Pyralidae (e.g., European corn borer (Ostrina nubilalis Hübner), navel orangeworm (Amyelois tracisilella Walker), corn root webworm (Crambus caliginosellus Clemens), sod webworm (Herpetogramma licarsisalis Walker)); leafrollers, budworms, seed worms, and fruit worms in the family Tortricidae (e.g., codling moth (Cydia pomonella Linnaeus) grape berry moth (Endopiza viteana Clemens), oriental fruit moth (Grapholita molesta Busck)); and many other economically important lepidoptera (e.g., diamondback moth (Plutella xylostella Linnaeus), pink bollworm (Pectinophora gossypiella Saunders), gypsy moth (Lymantria dispar Linnaeus)); nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blatella germianica Linnaeus), brownbanded cockroach (Supella lonigipalpa Fabricius), American cockroach (Periplanieta americana Linnaeus), brown cockroach (Periplaneta brunnea Burmeister), Madeira cockroach (Leticophaea maderae Fabricius)); foliar feeding larvae and adults of the order Coleoptera including weevils from the families Anthribidae, Bruchidae, and Curculionidae (e.g., boll weevil (Anthonomus gratidis Boheman), rice water weevil (Lissorhoptrus oryzophiltus Kuschel), granary weevil (Sitophilus granarios Linnaeus), rice weevil (Sitophilius oryzae Linnaeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scaribaeidae (e.g., Japanese beetle (Popillia japonica Newman) and European chafer (Rhizotrogus majalis Razoumowsky)); carpet beetles from the family Dermestidae; wireworms from the family Elateridae; bark beetles from the family Scolytidae and flour beetles from the family Tenebrionidae. In addition it includes: adults and larvae of the order Dermaptera including earwigs from the family Forficulidae (e.g., European earwig (Forficula auricularia Linnaeus), black earwig (Clhelisoches monio Fabricius)); adults and nymphs of the orders Hemiptera and Homoptera such as, plant bugs from the family Miridae, cicadas from the family Cicadidae, leafhoppers (e.g. Empoasca spp.) from the family Cicadellidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, cinch bugs (e.g., Blisszis spp.) and other seed bugs from the family Lygaeidae, spittlebugs from the family Cercopidae squash bugs from the family Coreidae, and red bugs and cotton stainers from the family Pyrrhocoridae. Also included are adults and larvae of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)), flat mites in the family Tenuipalpidae (e.g., citrus flat mite (Brevipalpus lewisi McGregor)), rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae (e.g., deer tick (Ixodes scapularis Say), Australian paralysis tick (Ixodes holocyclus Neumann), American dog tick (Dermacentor variabilis Say), lone star tick (Amblyomma americanum Linnaeus) and scab and itch mites in the families Psoroptidae, Pyemotidae, and Sarcoptidae; adults and immatures of the order Orthoptera including grasshoppers, locusts and crickets (e.g., migratory grasshoppers (e.g., Melanoplus sanguinipes Fabricius, M. differetialis Thomas), American grasshoppers (e.g., Schistocerca americania Drury), desert locust (Schistocerca gregaria Forskal), migratory locust (Locusta migratoria Linnaeus), house cricket (Acheta domesticus Linnaeus), mole crickets (Gryllotalpa spp.)); adults and immatures of the order Diptera including leafminers, midges, fruit flies (Tephritidae), frit flies (e.g., Oscinella frit Linnaeus), soil maggots, house flies (e.g., Musca domestica Linnaeus), lesser house flies (e.g., Fannia caninularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; adults and immatures of the order Thysanoptera including onion thrips (Thrips tabaci Lindeman) and other foliar feeding thrips; insect pests of the order Hymenoptera including ants (e.g., red carpenter ant (Camponotus ferrugineus Fabricius), black carpenter ant (Camponotus pennnsylvanicus DE Geer), Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Förster), odorous house ant (Tapinoma sessile Say)), bees (including carpenter bees), hornets, yellow jackets and wasps; insect pests of the order Isoptera including the eastern subterranean termite (Reticulitermes herperus Kollar), western subterranean termite (Rericulitermes hesperus Banks), Formosan subterranean termite (Coptotermes formosanus Shiraki), West Indian drywood termite (Incisitermes immigrans Snyder) and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus humanus Linnaeus), chicken body louse (Menacacthus stramineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; insect pests of the order Siphonoptera including the oriental rat flea (Xellopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus). Activity also includes members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongtlus vulgaris in horses, Toxocara canis in dogs, Haemonchus contortus in sheep, Dirofilaria immitis Leidy in dogs, Anoplocephala perfoliata in horses, Fasciola hepatica Linnaeus in ruminants, etc.).

Compounds of the invention show particularly high activity against pests in the order Lepidoptera (e.g., Alabama argillacea Hübner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenee (rice leaf roller), Crambus caliginiosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hübner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermüller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigita Hübner (beet armyworm), Spodoptera litura Fabricius (tobacco cutworm, cluster caterpillar), Spodoptera frugiperda J. E. Smith (fall armyworm), Trichoplusia ni Hübner (cabbage looper) and Tuta absoluta Meyrick (tomato leafminer)). Compounds of the invention also have commercially significant activity on members from the order Homoptera including: Acyrthisiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spirarcola Patch (spirea aphid), Aulacorthum solani Kaltenbach (foxglove aphid), Chaetosiphon fragarfolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip -aphid), Metopolophium dirrhodum Walker (cereal aphid), Macrosipum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graiminim Rondani (greenbug), Sitobion avenae Fabricius (English grain aphid), Therioaphis mactulata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows & Perring (silverleaf whitefly), Dialeurodes citri Ashmead (citrus whitefly) and Trialeurodes vaporarioirum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stal (rice leafhopper), Nilaparvata lugents Stål (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erythroneoura spp. (grape leafhoppers); Magicidada septenzclecim Linnaeus (periodical cicada); Icerya purchasi Maskell (cottony cushion scale), Quadraspidiotus perniciosus Comstock (San Jose scale); Planiococcus citri Risso (citrus mealybug); Pseudococus spp. (other mealybug complex); Cacopsylla pyricola Foerster (pear psylla), Trioza diospyri Ashmead (persimmon psylla). These compounds also have activity on members from the order Hemiptera including: Acrosternum hilare Say (green stink bug), Anasa tristis De Geer (squash bug), Blissus leucopterus leucopterus Say (chinch bug), Corythutca gossypii Fabricius (cotton lace bug), Cyrtopeltis modesta Distant (tomato bug), Dysdercus suturellus Herrich-Schäffer (cotton stainer), Eutchistus servus Say (brown stink bug), Euchistus variolarius Palisot de Beauvois (one-spotted stink bug), Graptosthetus spp. (complex of seed bugs), Leptoglossus corculus Say (leaf-footed pine seed bug), Lygus lineolaris Palisot de Beauvois (tarnished plant bug), Nezara virdula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the invention include Thysanoptera (e.g., Frankliniella occidenitalis Pergande (western flower thrip), Scirthothrops citri Moulton (citrus thrip), Sericothrips variabilis Beach (soybean thrip), and Thrips tabaci Lindeman (onion thrip); and the order Coleoptera (e.g., Leptinotarsa decemlineata Say (Colorado potato beetle), Epilachna varivestis Mulsant (Mexican bean beetle) and wireworms of the genera Agriotes, Atlous or Limonius).

Compounds of this invention can also be mixed with one or more other biologically active compounds or agents including insecticides, fungicides, nematocides, bactericides, acaricides, growth regulators such as rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agricultural utility. Thus compositions of the present invention can further comprise a biologically effective amount of at least one additional biologically active compound or agent. Examples of such biologically active compounds or agents with which compounds of this invention can be formulated are: insecticides such as abamectin, acephate, acetamiprid, avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, oxamyl, parathion, parathion-methyl, permethrin, phorate, phosalone, phosmet, phosphamidon, pirimicarb, profenofos, pymetrozine, pyridalyl, pyriproxyfen, rotenone, spinosad, sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (5)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metomino-strobin/fenominostrobin (SSF-126), myclobutanil, neo-asozin (ferric methanearsonate), oxadixyl, penconazole, pencycuron, probenazole, prochloraz, propamocarb, propiconazole, pyrifenox, pyraclostrobin, pyrimethanil, pyroquilon, quinoxyfen, spiroxamine, sulfur, tebuconazole, tetraconazole, thiabendazole, thifluzamide, thioplhanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents such as Bacillizs thutritigietisis including ssp. aizaivai and kzirstaki, Bacillus thuiriiigieiisis delta endotoxin, baculovirus, and entomopathogenic bacteria, virus and fungi.

A general reference for these agricultural protectants is The Pesticide Manual, 12th Edition, C. D. S. Tomlin, Ed., British Crop Protection Council, Farnham, Surrey, U.K., 2000.

Preferred insecticides and acaricides for mixing with compounds of this invention include pyrethroids such as cypermethrin, cyhalothrin, cyfluthrin, beta-cyfluthrin, esfenvalerate, fenvalerate and tralomethrin; carbamates such as fenothicarb, methomyl, oxamyl and thiodicarb; neonicotinoids such as clothianidin, imidacloprid and thiacloprid; neuronal sodium channel blockers such as indoxacarb; insecticidal macrocyclic lactones such as spinosad, abamectin, avenrnectin and emamectin; γ-aminobutyric acid (GABA) antagonists such as endosulfan, ethiprole and fipronil; insecticidal ureas such as flufenoxuron and triflumuron; juvenile hormone mimics such as diofenolan and pyriproxyfen; pymetrozine; and amitraz. Preferred biological agents for mixing with compounds of this invention include Bacillus thuringiensis and Bacillus thuringiensis delta endotoxin as well as naturally occurring and genetically modified viral insecticides including members of the family Baculoviridae as well as entomophagous fungi.

Most preferred mixtures include a mixture of a compound of this invention with cyhalothrin; a mixture of a compound of this invention with cyfluthrin; a mixture of a compound of this invention with beta-cyfluthrin; a mixture of a compound of this invention with esfenvalerate; a mixture of a compound of this invention with methomyl; a mixture of a compound of this invention with imidacloprid; a mixture of a compound of this invention with thiacloprid; a mixture of a compound of this invention with indoxacarb; a mixture of a compound of this invention with abamectin; a mixture of a compound of this invention with endosulfan; a mixture of a compound of this invention with ethiprole; a mixture of a compound of this invention with fipronil; a mixture of a compound of this invention with flufenoxuron; a mixture of a compound of this invention with pyriproxyfen; a mixture of a compound of this invention with pymetrozine; a mixture of a compound of this invention with amitrax; a mixture of a compound of this invention with Bacillus thuringiensis and a mixture of a compound of this invention with Bacillus thuringiensis delta endotoxin.

In certain instances, combinations with other invertebrate pest control compounds or agents having a similar spectrum of control but a different mode of action will be particularly advantageous for resistance management. Thus, compositions of the present invention can further comprise an biologically effective amount of at least one additional invertebrate pest control compounds or agents having a similar spectrum of control but a different mode of action. Contacting a plant genetically modified to express a plant protection compound (e.g., protein) or the locus of the plant with a biologically effective amount of a compound of invention can also provide a broader spectrum of plant protection and be advantageous for resistance management.

Invertebrate pests are controlled and protection of agronomic, horticultural and specialty crops, animal and human health is achieved by applying one or more of the compounds of this invention, in an effective amount, to the environment of the pests including the agronomic and/or nonagronomic locus of infestation, to the area to be protected, or directly on the pests to be controlled. Thus, the present invention further comprises a method for the control of foliar- and soil-inhabiting invertebrates and protection of agronomic and/or nonagronomic crops, comprising contacting the invertebrates or their environment with a biologically effective amount of one or more of the compounds of the invention, or with a composition comprising at least one such compound or a composition comprising at least one such compound and an effective amount of at least one additional biologically active compound or agent. A preferred method of contact is by spraying. Alternatively, a granular composition comprising a compound of the invention can be applied to the plant foliage or the soil. Compounds of this invention are effective in delivery through plant uptake by contacting the plant with a composition comprising a compound of this invention applied as a soil drench of a liquid formulation, a granular formulation to the soil, a nursery box treatment or a dip of transplants. Other methods of contact include application of a compound or a composition of the invention by direct and residual sprays, aerial sprays, seed coats, microencapsulations, systemic uptake, baits, eartags, boluses, floggers, fumigants, aerosols, dusts and many others.

The compounds of this invention can be incorporated into baits that are consumed by the invertebrates or within devices such as traps and the like. Granules or baits comprising between 0.01-5% active ingredient, 0.05-10% moisture retaining agent(s) and 40-99% vegetable flour are effective in controlling soil insects at very low application rates, particularly at doses of active ingredient that are lethal by ingestion rather than by direct contact.

The compounds of this invention can be applied in their pure state, but most often application will be of a formulation comprising one or more compounds with suitable carriers, diluents, and surfactants and possibly in combination with a food depending on the contemplated end use. A preferred method of application involves spraying a water dispersion or refined oil solution of the compounds. Combinations with spray oils, spray oil concentrations, spreader stickers, adjuvants, other solvents, and synergists such as piperonyl butoxide often enhance compound efficacy.

The rate of application required for effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredient per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required.

One skilled in the art can easily determine the biologically effective amount necessary for the desired level of invertebrate pest control. The following Tests in the Biological Examples of the Invention demonstrate the control efficacy of compounds of this invention on specific pests. “Control efficacy” represents inhibition of arthropod development (including mortality) that causes significantly reduced feeding. The pest control protection afforded by the compounds is not limited, however, to these species. See Index Tables A-E for compound descriptions. The following abbreviations are used in the Index Tables that follow: t is tertiary, si is normal, i is iso, s is secondary, Me is methyl, Et is ethyl, Pr is propyl and Bu is butyl; accordingly i-Pr is isopropyl, s-Bu is secondary butyl, etc. Ac is COCH₃. The abbreviation “Ex.” stands for “Example” and is followed by a number indicating in which example the compound is prepared.

INDEX TABLE A

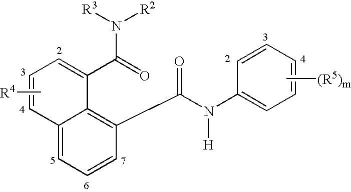

| Com- | | | | | | m.p. |
|---|---|---|---|---|---|---|
| pound | R² | R³ | R⁴ | m | R⁵ | ° C. |
| 1 (Ex. 1) | Me | H | H | 2 | 2-Me-4-F | 249 |
| 2 | i-Pr | H | H | 2 | 2-Me-4-Cl | >250 |
| 3 | n-Pr | H | H | 2 | 2-Me-4-Cl | 212 |
| 4 | i-Bu | H | H | 2 | 2-Me-4-Cl | *** |
| 5 | Me | Me | H | 2 | 2-Me-4-Cl | *** |
| 6 | Et | H | H | 2 | 2-Me-4-Cl | *** |
| 7 | Me | H | H | 2 | 2-Me-4-Cl | 244 |
| 8 | allyl | H | H | 2 | 2,4-di-Me | 243 |
| 9 | cyclo-Pr | H | H | 2 | 2-Me-4-Cl | 246 |
| 10 | H | H | H | 2 | 2-Me-4-Cl | *** |
| 11 | Me | H | H | 2 | 2-Me-4-OCF₃ | >250 |
| 12 | Me | H | H | 1 | 4-OCF₃ | >250 |
| 13 | Me | H | H | 2 | 2,4-di-Cl | >250 |
| 14 | Me | H | H | 2 | 2-Me-5-Cl | 241 |
| 15 | Me | H | H | 2 | 2-Me-3-Cl | >250 |
| 16 | Me | H | H | 1 | 2-Me | 236 |
| 17 | Me | H | H | 2 | 2-Me-4-Br | 234 |
| 18 | Me | H | H | 1 | 4-i-Pr | >250 |
| 19 | Me | H | H | 1 | 4-NHAc | 224 |
| 20 | Me | H | H | 1 | 2-Br | 210 |
| 21 | Me | H | H | 1 | 2-OPh | 193 |
| 22 | Me | H | H | 1 | 4-OMe | 188 |
| 23 | Me | H | H | 1 | 4- | 246 |
| | | | | | (morpholin-4-yl) | |
| 24 | Me | H | H | 1 | 2-F | 177 |
| 25 | Me | H | H | 1 | 4-I | 235 |
| 26 | Me | H | H | 2 | 2-Me-4-NO₂ | 242 |
| 27 | Me | H | H | 1 | 3-CF₃ | 235 |
| 28 | Me | H | H | 1 | 3-I | 216 |
| 29 | Me | H | H | 2 | 2-Me-4-OMe | 205 |
| 30 | Me | H | H | 1 | 3-Br | 230 |
| 31 | Me | H | H | 1 | 4-Ac | 226 |
| 32 | Me | H | H | 1 | 4-Br | 220 |
| 33 | Me | H | H | 1 | 4-CN | 216 |
| 34 | Me | H | H | 1 | 3-NO₂ | 242 |
| 35 | Me | H | H | 1 | 4-Cl | 197 |
| 36 | Me | H | H | 1 | 3-CH₃ | 220 |
| 37 | Me | H | H | 1 | 3-Cl | 209 |
| 38 | Me | H | H | 2 | 2,5-di-Me | 204 |
| 39 | Me | H | H | 2 | 2-Me-6-OMe | 223 |
| 40 | Me | H | H | 2 | 2,3-di-Me | 187 |
| 41 | Me | H | H | 1 | 2-OMe | 192 |
| 42 | Me | H | H | 1 | 4-CO₂Et | 202 |
| 43 | Me | H | H | 1 | 4-NMeAc | 210 |
| 44 | Me | H | H | 2 | 2,6-di-Me | >250 |
| 45 | Me | H | H | 1 | 2-(pyrrol-1-yl) | 208 |
| 46 | Me | H | H | 1 | 4-NO₂ | 224 |
| 47 | Me | H | H | 1 | 3-OCH₂Ph | 214 |
| 48 | Me | H | H | 2 | 2,4-di-Me | 213 |
| 49 | Me | H | H | 1 | 3-CN | 200 |
| 50 | Me | H | H | 1 | 3-OCF₃ | >250 |
| 51 | Me | H | H | 2 | 2-Me-3-NO₂ | 233 |
| 52 | Me | H | H | 1 | 2-OEt | 224 |
| 53 | Me | H | H | 1 | 4-(4-ClPhO) | 224 |
| 54 | Me | H | H | 1 | 4-CH₃ | 248 |
| 55 | Me | H | H | 1 | 4-O-i-Pr | 183 |
| 56 | Me | H | H | 1 | 4-F | 247 |
| 57 | Me | H | H | 1 | 2-SMe | 204 |
| 58 | Me | H | H | 1 | 2-t-Bu | 248 |
| 59 | Me | H | H | 1 | 3-SMe | 218 |
| 60 | Me | H | H | 1 | 4-OEt | 195 |
| 61 | Me | H | H | 1 | 2-CH₂CN | 191 |
| 62 | Me | H | H | 1 | 2-Et | 200 |
| 63 | i-Pr | H | 4-NO2/ | 2 | 2-Me-4-Cl | *** |
| | | | 5-NO2 | | | |
| | | | (1/1 mixture) | | | |
| 64 | Me | H | 4-Br/ | 2 | 2-Me-4-Cl | *** |
| | | | 5-Br | | | |
| | | | (1/1 mixture) | | | |
| 65 | Me | H | H | 1 | 2-CONH(i-Pr)- | 248 |
| | | | | | 6-Me | |
| 66* | Me | H | H | 2 | 2-Me-4-Cl | *** |
| 67* | Me | H | H | 2 | 2-Me-4-OCF₃ | *** |
| 68 | Me | H | H | 2 | 2-Me-S-NO₂ | 242 |
| 69 | Me | H | H | 2 | 2-Me-4-CF₃ | 250 |
| 70 | Me | H | H | 1 | 4-CF₃ | 246 |
| 71 | Me | H | H | 1 | 2-Ph | 163 |
| 72 | Me | H | H | 2 | 2-(1-Me-tetrazol- | 202 |
| | | | | | 5-yl)-6-Me | |
| 73 | Me | H | H | 1 | 2-CONHMe | 235 |
| 74 | Me | H | H | 2 | 2-Me-6-Cl | 231 |
| 75 | Me | H | H | 2 | 3-NO₂-4-Me | 223 |
| 76 | Me | H | H | 3 | 2-F-4-Cl-5-NO₂ | 250 |
| 77 | Me | H | H | 2 | 2-Br-4-Me | 198 |
| 78 | Me | H | H | 3 | 2-Br-4,6-di-F | 197 |
| 79 | Me | H | H | 2 | 2,5-di-NO₂ | 223 |
| 80 | Me | H | H | 2 | 2-F-4-Cl | 250 |
| 81 | Me | H | H | 2 | 2-Me-3-F | 242 |
| 82 | Me | H | H | 2 | 2-Br-4-F | 126.5 |
| 83 | Me | H | H | 1 | 2- | 193 |
| | | | | | (morpholin-4-yl) | |
| 84 | Me | H | H | 2 | 3-NO₂-4-F | 248 |
| 85 | Me | H | H | 2 | 3,4-di-F | 110 |
| 86 | Me | H | H | 2 | 2-Cl-4-F | 250 |
| 87 | Me | H | H | 1 | 2-CF₃ | 217 |
| 88 | Me | H | H | 2 | 2-CN-4-Cl | 250 |
| 89 | Me | H | H | 1 | 2-Ac | 237 |
| 90 | Me | H | H | 1 | 2-OCF₃ | 209 |
| 91 | Me | H | H | 1 | 2-SCF₃ | 211 |
| 92 | Me | H | H | 2 | 2-CF₃-4-Br | 250 |

*This sample contained 25% by weight of 1,8-naphthalene-anhydride.
***See Index Table E for NMR data.

INDEX TABLE B

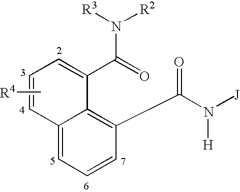

| Compound | R² | R³ | R⁴ | J | m.p. ° C. |
|---|---|---|---|---|---|
| 93 | Me | H | H | 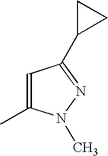 | 199 |
| 94 | Me | H | H | 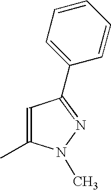 | 175 |
| 95 | Me | H | H | 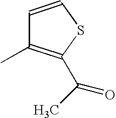 | 126.5 |
| 96 | Me | H | H | 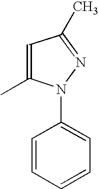 | 217 |
| 97 | Me | H | H | 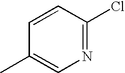 | 171 |

INDEX TABLE C

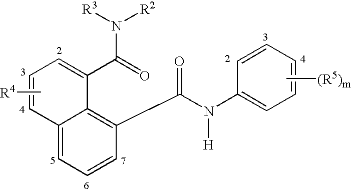

| Compound | R² | R³ | R⁴ | m | R⁵ | m.p. ° C. |
|---|---|---|---|---|---|---|
| 98 (Ex. 2) | i-Pr | H | H | 2 | 3,4-di-F | 227 |
| 99 | i-Pr | H | H | 2 | 2,3-di-Me | 242 |
| 100 | i-Pr | H | H | 2 | 2,4-di-F | 204 |
| 101 | i-Pr | H | H | 1 | 4-OCF₃ | 186 |
| 102 | i-Pr | H | H | 1 | 4-Cl | 218 |
| 103 | i-Pr | H | H | 1 | 4-Et | 206 |
| 104 | i-Pr | H | H | 1 | 4-CF₃ | 165 |
| 105 | Me | H | H | 2 | 3,4-di-F | 250 |
| 106 | Me | H | H | 2 | 2,3-di-Me | 184.5 |
| 107 | Me | H | H | 2 | 2,4-di-F | 207 |
| 108 | Me | H | H | 1 | 4-Cl | 250 |
| 109 | Me | H | H | 1 | 4-Et | 250 |
| 110 | Me | H | H | 1 | 4-CF₃ | 208 |
| 111 | Me | H | H | 1 | 4-OCF₃ | 144 |
| 112 | Me | H | H | 1 | 2-Me | 173.5 |
| 113 | i-Pr | H | H | 1 | 2-Me | 176.5 |
| 114 | i-Pr | H | H | 2 | 2-Me-4-O-CF₃ | 173.5 |
| 115 | i-Pr | H | H | 2 | 2-Me-4-CF₃ | 194.5 |
| 116 | Me | H | H | 2 | 2-Me-4-OCF₃ | 173.5 |
| 117 | Me | H | H | 2 | 2-Me-4-CF₃ | 204.5 |

INDEX TABLE D

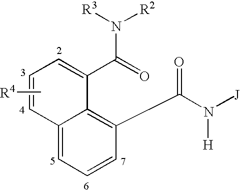

| Com- | | | | | |
|---|---|---|---|---|---|
| pound | R² | R³ | R⁴ | J | m.p. ° C. |
| 118 | i-Pr | H | H | 1-naphthyl | 210 |
| 119 | i-Pr | H | H | 2-thienyl | 208 |
| 120 | Me | H | H | 2-thienyl | 215 |
| (Ex. 3) | | | | | |
| 121 | Me | H | H | 1-naphthyl | 214 |
| 121 | i-Pr | H | H | 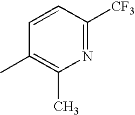 | 210 |
| 123 | Me | H | H | 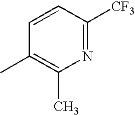 | 210 |
| 124 | Me | H | H | 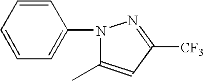 | 215.5 |
| 125 | i-Pr | H | H | 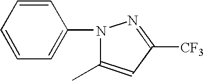 | 186 |

INDEX TABLE E

| Cmpd No. | ¹H NMR Data (CDCl₃ solution unless indicated otherwise)^{a} |
|---|---|
| 4 | (DMSO-d6) δ: 0.9 (m, 6H), 2.37 (s, 3H), 3.01 (m, 1H), 3.96 |
| | (d, 2H), 7.27-8.48 (m, 10H), 9.84 (br s, 1H). |
| 5 | (DMSO-d6) δ: 2.36 (s, 3H), 2.88 (s, 3H), 2.94 (s, 3H), |
| | 7.28-8.08 (m, 10H), 9.99 (s, 1H). |
| 6 | (DMSO-d6) δ: 1.07 (t, 3H), 2.37 (s, 3H), 3.22 (m, 2H), |
| | 7.30-8.06 (m, 9H), 8.40 (t, 1H), 9.86 (s, 1H). |
| 10 | (DMSO-d6) δ: 2.36 (s, 3K), 7.25-8.08 (m, 11H), 9.88 |
| | (s, 1H). |
| 63 | (DMSO-d6) δ: 1.09 (d, 6H), 2.36 (s, 3H), 4.03 (m, 1H), |
| | 7.27-8.52 (m, 9H), 10.04 (s, 1H). |
| 64 | (DMSO-d6) δ: 2.36 (s, 3H), 2.69 (d, 3H), 7.33-8.84 |
| | (m, 8H), 8.45 (br q, 1H), 9,98 (s, 1H). |
| 66 | (DMSO-d6) δ: 2.37 (s, 3H), 2.71 (d, 3H), 7.27-8.10 |
| | (m, 9H), 8.34 (br q, 1H), 9.88 (s, 1H). |
| 67 | (DMSO-d6) δ: 2.41 (s, 3H), 2.72 (d, 3H), 7.26-8.08 (m, |
| | 9H), 8.36 (br q, 1H), 9.95 (s, 1H). |

^{a}¹H NMR data are in ppm downfield from tetramethylsilane. Couplings are designated by (s)-singlet, (d)-doublet, (t)-triplet, (q)-quartet, (m)-multiplet, (dd)-doublet of doublets, (dt)-doublet of triplets, (br s)-broad singlet, (bs q)-broad quartet.

### BIOLOGICAL EXAMPLES OF THE INVENTION

### Test A

For evaluating control of fall armyworm (Spodoptera frugiperda) the test unit consisted of a small open container with a 45-day-old corn (maize) plant inside. This was pre-infested with 10-15 1-day-old larvae on a piece of insect diet by use of a core sampler to remove a plug from a sheet of hardened insect diet having many larvae growing on it and transfer the plug containing larvae and diet to the test unit. The larvae moved onto the test plant as the diet plug dried out.

Test compounds were formulated using a solution containing 10% acetone, 90% water and 300 ppm X-770 Spreader Lo-Foam Formula non-ionic surfactant containing alkylarylpolyoxyethylene, free fatty acids, glycols and isopropanol (Loveland Industries, Inc.), unless otherwise indicated. The formulated compounds were applied in 1 mL of liquid through a SUJ2 atomizer nozzle with 1/8 JJ custom body (Spraying Systems Co,) positioned 1.27 cm (0.5 inches) above the top of each test unit. All experimental compounds in this screen were sprayed at 250 ppm and replicated three times. After spraying of the formulated test compound, each test unit was allowed to dry for 1 hour and then a black, screened cap was placed on top. The test units were held for 6 days in a growth chamber at 25° C. and 70% relative humidity. Plant feeding damage was then visually assessed.

Of the compounds tested, the following provided excellent levels of plant protection (10% or less feeding damage): 1, 7, 11, 16, 48, 66 and 67.

### Test B

For evaluating control of tobacco budworm (Heliothis virescens) the test unit consisted of a small open container with a 67 day old cotton plant inside. This was pre-infested with 82-day-old larvae on a piece of insect diet by use of a core sampler as described for Test A.

Test compounds were formulated and sprayed at 250 ppm as described for Test A. The applications were replicated three times. After spraying, the test units were maintained in a growth chamber and then visually rated as described for Test A.

Of the compounds tested, the following provided excellent levels of plant protection (10% or less feeding damage): 1, 7 and 16.

### Test C

For evaluating control of diamondback moth (Plutella xylostella) the test unit consisted of a small open container with a 12-14-day-old radish plant inside. This was pre-infested with 10-15 neonate larvae on a piece of insect diet by use of a core sampler as described for Test A.

Test compounds were formulated and sprayed at 250 ppm as described for Test A. The applications were replicated three times. After spraying, the test units were maintained in a growth chamber and then visually rated as described for Test A.

Of the compounds tested, the following provided excellent levels of plant protection (10% or less feeding damage): 1, 7, 11, 12, 14, 16, 20, 24, 37, 48, 51, 54, 55, 56, 61, 66, 67, 69, 70, 72, 75, 81, 85, 86, 89, 90, 91, 93 and 97.

### Test D

For evaluating control of beet armyworm (Spodoptera exigua) the test unit consisted of a small open container with a 4-5-day-old corn plant inside. This was pre-infested with 10-15 1-day-old larvae on a piece of insect diet by use of a core sampler as described for Test A.

Test compounds were formulated and sprayed at 250 ppm as described for Test A. The applications were replicated three times. After spraying, the test units were maintained in a growth chamber and then visually rated as described for Test A.

Of the compounds tested, the following provided excellent levels of plant protection (10% or less feeding damage): 1, 40 and 54.

What is claimed is:

1. A compound selected from Formula I or Formula II, and N-oxides and agriculturally suitable salts thereof, 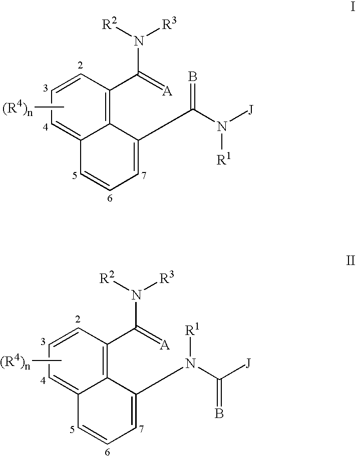
wherein
each J is independently a phenyl ring, a naphthyl ring system, a 5- or 6-membered heteroaromatic ring or an aromatic 8-, 9- or 10-membered fused heterobicyclic ring system wherein each ring or ring system is optionally substituted with 1 to 5 R⁵;
A and B are independently O or S;
n is 0 to 4;
R¹ is H; or C₁-C₆ alkyl, C₂-C₆ alkenyl, C₂-C₆ alkynyl or C₃-C₆ cycloalkyl each optionally substituted with one or more substituents selected from the group consisting of halogen, CN, NO₂, hydroxy, C₁-C₄ alkoxy, C₁-C₄ alkylthio, C₁-C₄ alkylsulfinyl, C₁-C₄ alkylsulfonyl, C₂-C₄ alkoxycarbonyl, C₁-C₄ alkylamino, C₂-C₈ dialkylamino and C₃-C₆ cycloalkylamino; or
R¹ is C₂-C₆ alkylcarbonyl, C₂-C₆ alkoxycarbonyl, C₂-C₆ alkylaminocarbonyl or C₃-C₈ dialkylaminocarbonyl;
R² is H, C₁-C₆ alkyl, C₂-C₆ alkenyl, C₂-C₆ alkynyl, C₃-C₆ cycloalkyl, C₁-C₄ alkoxy, C₁-C₄ alkylamino, C₂-C₈ dialkylamino, C₃-C₆ cycloalkylamino, C₂-C₆ alkoxycarbonyl or C₂-C₆ alkylcarbonyl;
R³ is H; or C₁-C₆ alkyl, C₂-C₆ alkenyl, C₂-C₆ alkynyl or C₃-C₆ cycloalkyl each optionally substituted with one or more substituents selected from the group consisting of halogen, CN, NO—, hydroxy, C₁-C₄ alkoxy, C₁-C₄ alkylthio, C₁-C₄ alkylsultinyl and C₁-C₄ alkylsulfonyl; or
R² and R³ can be taken together with the nitrogen to which they are attached to form a ring containing 2 to 6 atoms of carbon and optionally one additional atom of nitrogen, sulfur or oxygen, said ring may be optionally substituted with 1 to 4 substituents selected from the group consisting of C₁-C₂ alkyl, halogen, CN, NO— and C₁-C₂ alkoxy; and
each R⁴ and each R⁵ is independently H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl, halogen, CN, CO₂H, CONH₂, NO₂, hydroxy, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₂-C₄ alkoxycarbonyl, C₁-C₄ alkylamino, C₂-C₈ dialkylamino, C₃-C₆ cycloalkylamino, C₂-C₆ alkylcarbonyl, C₂-C₆ alkoxycarbonyl, C₂-C₆ alkylaminocarbonyl, C₃-C₈ dialkylaminocarbonyl, or C₃-C₆ trialkylsilyl; or
each R⁴ and each R⁵ is independently a phenyl, benzyl, phenoxy, 5- or 6-membered heteroaromatic ring or an aromatic 8-, 9- or 10-membered fused heterobicyclic ring system, each ring optionally substituted with one to three substituents independently selected from the group consisting of C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; or
(R⁵)₂ when attached to adjacent carbon atoms can be taken together as —OCF₂O—, —CF₂CF₂O— or —OCF₂CF₂O—.

2. The compound of Formula I of claim 1 wherein
A and B are both O;
R¹ is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl; and
n is 0 to 2.

3. The compound of Formula II of claim 1 wherein
A and B are both O;
R¹ is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl; and
n is 0 to 2.

4. The compound of claim 2 or claim 3 wherein
J is a phenyl ring or a 5- or 6-membered heteroaromatic ring selected from the group consisting of J-1, J-2, J-3 and J-4, each J ring optionally substituted with 1 to 3 R⁵ 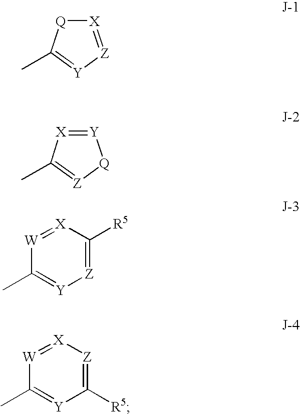
Q is O, S or NR⁵;
W, X, Y and Z are independently N or CR⁵, provided that in J-3 and J-4 at least one of W, X, Y or Z is N;
R² is H, C₁-C₄ alkyl, C₂-C₄ alkenyl, C₂-C₄ alkynyl, C₃-C₆ cycloalkyl, C₂-C₆ alkylcarbonyl or C₂-C₆ alkoxycarbonyl;
R³ is C₁-C₆ alkyl, C₂-C₆ alkenyl, C₂-C₆ alkynyl or C₃-C₆ cycloalkyl each optionally substituted with one or more substituents selected from the group consisting of halogen, CN, C₁-C₂ alkoxy, C₁-C₂ alkylthio, C₁-C₂ alkylsulfinyl and C₁-C₂ alkylsulfonyl;
one R⁴ group is attached to the naphthyl ring system at the 2-position or 7-position, and said R⁴ is C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl or C₁-C₄ haloalkylsulfonyl;
each R⁵ is independently H, C₁-C₄ alkyl, C₀-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₀-C₄ haloalkylthio, C₀-C₄ haloalkylsulfinyl, C₁-C₄ haloalkylsulfonyl or C₂-C₄ alkoxycarbonyl, C₃-C₈ dialkylaminocarbonyl; or
each R⁵ is independently a phenyl, benzyl or a 5- or 6-membered heteroaromatic ring, each ring optionally substituted with C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; or
(R⁵)₂ when attached to adjacent carbon atoms can be taken together as —OCF₂O—, —CF₂CF₂O— or —OCF₂CF₂O—; and
n is 1 to 2.

5. The compound of Formula I of claim 4 wherein R² is H; R³ is C₁-C₄ alkyl; and at least one of the R⁵ substituents is ortho to the NR¹C(═B) moiety.

6. The compound of claim 5 wherein R³ is methyl.

7. The compound of Formula II of claim 4 wherein
R is H or C₀-C₄ alkyl;
R² is H or C₁-C₄ alkyl;
R³ is C₁-C₄ alkyl optionally substituted with halogen, CN, OCH₃, or S(O)ₚCH₃;
one R⁵ group is attached to the J at the position ortho to the C(═B)NR¹ moiety, and said R⁵ is C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl; C₃-C₈ dialkylaminocarbonyl or a phenyl, benzyl, or a 5- or 6-membered heteroaromatic ring, each ring optionally substituted with halogen, CN, NO₂, C₁-C₄ alkyl, C₂-C₄ alkenyl, C₂-C₄ alkynyl, C₃-C₆ cycloalkyl, C₁-C₄ haloalkyl, C₁-C₄ alkoxy or C₁-C₄ haloalkoxy;
and a second optional R⁵ group is independently C₁-C₄ alkyl, C₁-C₄ haloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl or C₂-C₄ alkoxycarbonyl; C₃-C₄ dialkylaminocarbonyl or a phenyl, benzyl, or a 5- or 6-membered heteroaromatic ring, each ring optionally substituted with halogen, CN, NO₂, C₁-C₄ alkyl, C₂-C₄ alkenyl, C₂-C₄ alkynyl, C₃-C₆ cycloalkyl, C₁-C₄ haloalkyl, C₁-C₄ alkoxy or C₁-C₄ haloalkoxy.

7. The compound of claim 6 wherein J is phenyl, pyrazole, pyrrole, pyridine or pyrimidine, each substituted with one R⁵ attached to the J at the position ortho to the C(═B)NR¹ moiety and a second optional R⁵.

8. The compound of claim 7 wherein
R¹ and R² are each H;
one R⁴ is attached at the 7-position ortho to the NR¹ C(═X)J moiety and is selected from the group consisting of C₁-C₃ alkyl, CF₃, OCF₃, OCHF₂, S(O)ₚCF₃, S(O)ₚCHF₂ and halogen and an optional second R⁴ is attached at the 5-position para to the NR¹C(—X)J moiety and is selected from the group consisting of halogen, C₁-C₃ alkyl and C₁-C₃ haloalkyl.

9. The compound of claim 8 wherein
J is J-1;
Q is NR^{5a};
X is N or CH;
Y is CH;
Z is CR^{5b};
R^{5a} is a phenyl or 2-pyridyl ring substituted with one or two substituents selected from the group consisting of halogen, C₁-C₄ alkyl, C₁-C₄ haloalkyl or C₁-C₄ haloalkoxy; and
R^{5b} is halogen or CF₃.

10. The compound of claim 1 selected from the group consisting of
N-methyl —N′-(2-bromo-4-fluoropheny)-1,8-naphthalene-dicarboxamide,
N-methyl 8-[(3,4-di fluorophenyl)carbonylamino]-1-naphthalenecarboxamide and
N-methyl 8-[(2-thienyl)carbonylamino]-1-naphthalenecarboxamide.

11. A composition for controlling an invertebrate pest comprising a biologically effective amount of a compound of claim 1 and at least one of a surfactant, a solid diluent or a liquid diluent.

12. The composition of claim 1 further comprising an effective amount of at least one additional biologically active compound or agent.

13. The composition of claim 12 wherein at least one additional biologically active compound or agent is selected from arthropodicides of the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics.

14. The composition of claim 12 wherein at least one additional biologically active compound or agent is selected from insecticide, nematocide, acaricide or biological agents in the group consisting of abamectin, acephate, acetamiprid, avermnectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, oxamyl, parathion, parathion-methyl, pernmethrin, phorate, phosalone, phosmet, phosphamidon, pirimicarb, profenofos, pymetrozine, pyridalyl, pyriproxyfen, rotenone, spinosad, sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad, Bacillus thuringiensis, Bacillus thuringiensis delta endotoxin, baculovirus, and entomopathogenic bacteria, virus and fungi.

15. The composition of claim 14 wherein at least one additional biologically active compound or agent is selected from the group consisting of cypermethrin, cyhalothrin, cyfluthrin, beta-cyfluthrin, esfenvalerate, fenvalerate, traloniethrin, fenothicarb, methomyl, oxamyl, tliiodicarb, clothianidin, imidacloprid, thiacloprid, indoxacarb, spinosad, abamectin, avermectin, emamectin, endosulfan, ethiprole, fipronil, flufenoxuron, triflumuron, diofenolan, pyriproxyfen, pymetrozine, amitraz, Bacillus thuringiensis, Bacillus thuringiensis delta endotoxin and entomophagous fungi.

16. A method for controlling an invertebrate pest comprising contacting the invertebrate pest or its environment with a biologically effective amount of a compound of claim 1 or a composition of claim 11.
